(19) United States
(12) Patent Application Publication
(10) Pub. No.: US 2024/0102102 A1
(43) Pub. Date: Mar. 28, 2024

(54) METHODS FOR DETECTING utDNA

(71) Applicant: Washington University, St. Louis, MO (US)

(72) Inventors: Aadel Chaudhuri, St. Louis, MO (US); Kevin Chen, St. Louis, MO (US); Pradeep Chauhan, St. Louis, MO (US); Zachary Smith, St. Louis, MO (US); Vivek Arora, St. Louis, MO (US)

(73) Assignee: Washington University, St. Louis, MO (US)

(21) Appl. No.: 18/261,874

(22) PCT Filed: Jan. 18, 2022
(86) PCT No.: PCT/US2022/012748

§ 371 Date: Jul. 18, 2023

Related U.S. Application Data
(60) Provisional application No. 63/138,705, filed on Jan. 18, 2021.

(51) Int. Cl. C12Q 1/6886; C12Q 1/6874

(52) U.S. Cl. CPC C12Q 1/6886; C12Q 1/6874; C12Q 1/6806; C12Q 2600/106; C12Q 2600/156

(57) ABSTRACT

Among the various aspects of the present disclosure is the provision of treatments, monitoring treatment or progression, or methods for detecting utDNA in a subject having or suspected of having a urinary tract-associated cancer.

### CROSS-REFERENCE TO RELATED APPLICATIONS

This application claims priority from U.S. Provisional Application Ser. No. 63/138,705 filed on Jan. 18, 2021, which is incorporated herein by reference in its entirety.

### STATEMENT REGARDING FEDERALLY SPONSORED RESEARCH OR DEVELOPMENT

This invention was made with government support under TR002345 and CA238711 awarded by the National Institutes of Health. The government has certain rights in the invention.

### MATERIAL INCORPORATED-BY-REFERENCE

Not applicable.

### FIELD

The present disclosure generally relates to methods of detecting minimal residual disease in cancer patients.

### SUMMARY

Among the various aspects of the present disclosure is the provision of methods for detecting utDNA in a subject having or suspected of having a urinary tract-associated cancer, such as bladder cancer. Various aspects of the present disclosure provide for treatment, monitoring treatment or progression, or methods for detecting utDNA in a subject having or suspected of having a urinary tract-associated cancer. An aspect of the present disclosure provides for a method of detecting urine tumor DNA (utDNA) in a subject having or suspected of having a urinary tract-associated cancer comprising: providing or having been provided a biological sample comprising urine or derived from urine from the subject, and/or detecting utDNA in the biological sample. In some embodiments, the utDNA detected are mutations in MRD-associated genes or TMB-associated genes. In some embodiments, the mutations in the MRD-associated genes or TMB-associated genes are non-silent mutations. In some embodiments, the subject is suspected of having a urinary tract-associated cancer or is determined to need further testing if utDNA levels are above 0%. In some embodiments, the utDNA is DNA associated with minimal residual disease (MRD) in a subject that previously received or is receiving neoadjuvant therapy. In some embodiments, the utDNA is detected in the biological sample of the subject after neoadjuvant chemotherapy but prior to surgery. In some embodiments, the level of utDNA correlates with pathologic response to the neoadjuvant therapy. In some embodiments, the utDNA detected are mutations, optionally non-silent mutations, in MRD-associated genes selected from: TERT or TP53 or both. In some embodiments, the utDNA detected are mutations, optionally non-silent mutations, in MRD-associated genes selected from: ARID1A; CDKN1A; ERCC5; KMT2C; POLE; TP53; ASXL2; CDKN2A; FANCC; KMT2D; RB1; TSC1; ATM; CREBBP; FBXW7; KRAS; RHOA; TXNIP; ATR; ELF3; FGFR3; MDM2; RHOB; ZFP36L1; BRCA1; EP300; FOXA1; NF1; RXRA; BRCA2; ERBB2; FOXQ1; NFE2L2; SPTAN1; BTG2; ERBB3; HRAS; PAIP1; STAG2; CCND1; ERBB4; KDM6A; PIK3CA; TBC1D12; CCND3; ERCC2; KLF5; PLEKHS1; TERT; or combinations thereof. In some embodiments, the method comprises determining if a subject is utDNA MRD-positive or utDNA MRD-negative. In some embodiments, if one or more mutations or non-silent mutations in MRD-associated genes are detected, the subject is considered utDNA MRD-positive. In some embodiments, if no mutations or non-silent mutations in the MRD-associated genes are detected, the subject is considered utDNA MRD-negative. In some embodiments, the method comprises calculating a threshold using utDNA values from healthy adults or pathologic complete response (pCR) subjects or subjects with a utDNA level above or below this threshold were designated as utDNA MRD-positive or utDNA MRD-negative, respectively. In some embodiments, the subject is utDNA MRD-positive and no pCR if utDNA level is at or above 2.3%; or the subject is utDNA MRD-negative and pCR if utDNA level is below 2.3%. In some embodiments, the biological sample comprises a urine cell-free DNA (cfDNA) sample. In some embodiments, the urinary tract-associated cancer is a: a cancer of the urinary tract, a bladder cancer, such as localized bladder cancer, metastatic bladder cancer, muscle-invasive bladder cancer (MIBC), non-muscle invasive bladder cancer (NMIBC), urothelial carcinoma, papillary urothelial carcinoma, squamous cell carcinoma, or urothelial carcinoma with trophoblastic differentiation, including choriocarcinomatous differentiation; a urothelial carcinoma in the urinary tract; a genitourinary cancer, such as bladder cancer, kidney cancer, testicular cancer, urethral cancer, prostate cancer, penile cancer, ureteral cancer, urethral cancer, or adrenal cancer; a genitourinary cancer selected from adrenal cancer (e.g., adrenocortical carcinoma), bladder cancer (e.g., urothelial carcinoma, urachal cancer), kidney cancer (e.g., collecting duct carcinoma, renal cell carcinoma, rhabdoid tumor of the kidney, renal pelvic cancer, Wilms tumor (nephroblastoma)), penile cancer (e.g., urethral cancer), prostate cancer, or testicular cancer (germ cell tumor) (e.g., seminoma, non-seminoma, such as choriocarcinoma proud or teratoma); or kidney cancer, such as renal pelvis cancer, renal cell carcinoma, sarcomatoid (e.g., clear cell, papillary, chromophobe), medullary, collecting duct, urothelial carcinoma, sarcoma, Wilms tumor, lymphoma, oncocytoma, or angiomyolipoma. In some embodiments, inferred tumor mutational burden (iTMB) is determined from TMB-associated utDNA, wherein the subject is optionally MRD-positive or MRD-negative. In some embodiments, the TMB-associated utDNA detected are mutations, optionally non-silent mutations, in TMB-associated genes selected from: ABCA6; C16orf71; COLGALT2; ERBB3; HCG4P5; KCNH6; ABCC5; C1QTNF1; COPB2; ERBB4; HCN1; KCTD19; ACAA1; C3orf17; COPG1; ERCC2; HECW1; KDM3A; ACPP; C3orf20; CPD; ERCC5; HELZ; KDM6A; ACTL8; C3orf58; CPSF6; FAM71A; HERC1; KIAA0100; ADAMTS12; C3orf70; CRB1; FAM71F1; HEXIM1; KIAA0556; ADAMTS16; C9orf156; CREBBP; FANCC; HHIP; KIAA1045; ADRA1A; C9orf64; CRTC2; FANCM; HHLA2; KLF5; AHR; CACNA1A; CRYAB; FBXL7; HIST1H1B; KLF6; AKT1; CACNA2D4; CSN3; FBXO42; HIST1H1C; KMT2C; ALAS1; CACNG4; CSPG5; FBXW7; HIST1H1E; KMT2D; ALDH16A1; CADM2; CTNNA2; FCRL3; HIST1H2AG; KRAS; ALYREF; CARD11; CTNNB1; FERD3L; HIST1H2AK; KRIT1; AMACR; CARD14; CTNND2; FETUB; HIST1H2BG; LAX1; AMIGO3; CBLB; CTTN; FFAR4; HIST1H2BH; LHFPL5; ANAPC11; CCDC129; CUBN; FGFR1; HIST1H2BI; LILRA2; ANKRD28; CCDC173; CUL1; FGFR2; HIST1H2BK; LILRB4; ANO4; CCDC36; CYP7A1; FGFR3; HIST1H3B; LIPH; APOA4; CCDC96; DACH1; FLRT2; HIST1H3F; LPAR6; AQP7; CCND1; DCAF12L2; FOXA1; HIST1H3H; LRGUK; ARHGEF16; CCND3; DCAF4L2; FOXA3; HIST1H31; LRRC7; ARID1A; CCNE1; DDX42; FOXQ1; HIST1H4D; LRRK2; ARID4A; CCR5; DGCR8; FURIN; HIST1H41; LRRN1; ASH1L; CD1D; DGKG; FZD6; HIST1H4L; LRRTM1; ASIC2; CD5L; DHPS; GABRA1; HLA-A; LRTM2; ASXL2; CD86; DICER1; GABRB1; HLA-DQB2; MAG11; ATM; CDC27; DIDO1; GABRG1; HNRNPA2B1; MAG12; ATP10A; CDH1; DLC1; GALNT8; HOXA13; MAP1LC3A; ATP8B4; CDH10; DLEC1; GALR2; HOXA7; MAPK8; ATR; CDH6; DLGAP1; GAREM; HOXB1; MAPKAPK3; ATXN2L; CDK12; DLGAP5; GATA3; HOXB3; MARCHF1; AUTS2; CDK6; DNAH10; GCNT2; HOXB4; MB21D2; BCAS3; CDKN1A; DNAH11; GEMIN2; HRAS; MCL1; BCL2L1; CDKN2A; DNAH5; GHSR; HRH1; MCM7; BIRC6; CDKN2B; DNTT; GL13; HSPB2; MDM2; BMS1; CEP63; DOPEY2; GMPPB; HSPB2-C11orf52; MED12; BPIFB4; CEP89; DPYS; GNA13; HTR1A; MED13; BRAF; CFL1; DSC1; GNAT1; HTRA2; MET; BRCA1; CHD2; DTX1; GNGT1; ID1; METTL3; BRCA2; CLASP2; E2F3; GOLGA1; IDH1; MGAT5B; BRINP3; CLCNKB; EEF1B2; GPC5; IDH2; MIOS; BRIP1; CLIP1; EFCAB5; GPC6; IKZF2; MIR1306; BRMS1; CLVS2; EGFR; GRIK3; IRX1; MIR4728; BTG2; CNKSR3; EIF6; GRM7; ITGA3; MLIP; C10orf90; CNTN1; ELF3; GSS; ITIH3; MMP25; C11orf16; CNTN6; EMILIN2; GTPBP8; JAK2; MMP9; C11orf65; CNTNAP1; EP300; GUCY1A3; KARS; MOCS1; C12orf43; COL11A2; EPHA3; GYS1; KCNA1; MPHOSPH8; C12orf66; COL1A2; EPHB4; H1FOO; KCNB2; MROH2B; C15orf52; COL6A3; ERBB2; HARB11; KCND2; MRPS22; MS4A3; PCDHGA5; RBM47; SUMF1; WAC; MTAP; PCDHGB1; RBP4; SYNE1; WDR64; MTOR; PCDHGB2; RECQL4; SYNE2; WNT7A; MYC; PCDP1; RECQL5; SYT10; XKR7; MYCL; PCF11; RHOA; SYT15; YAE1 D1; MYF5; PCK1; RHOB; T; YAP1; MYH3; PCSK6; RHOU; TADA2B; YWHAZ; MYH7; PDE1C; RIPK4; TBC1D12; ZACN; MYO3A; PDE3A; RIT2; TBC1D16; ZBTB22; NAB2; PDE4DIP; RNASE3; TBKBP1; ZBTB45; NACA2; PEG3; RNF10; TCFL5; ZBTB7B; NANOS3; PGAP3; RNF111; TENM3; ZFC3H1; NCKAP5; PHACTR1; RNF123; TERT; ZFHX4; NCOR1; PHLDA3; RNF213; TFEC; ZFP36L1; NDUFAF6; PIGS; RNF216; TFP12; ZFP36L2; NEURL4; PIK3CA; RNF220; TGIF2LX; ZFYVE1; NF1; PKHD1; ROBO2; THAP2; ZFYVE9; NFE2L2; PKHD1L1; RTN4RL2; THSD7B; ZIC1; NKIRAS2; PLAUR; RUSC1; TIE1; ZIM2; NKTR; PLEKHA7; RXRA; TMCO2; ZIM3; NKX2-2; PLEKHS1; RYR2; TMCO4; ZNF263; NLRP8; POLDIP2; S1PR1; TMEM131; ZNF276; NOM1; POLE; SAMD4B; TMEM143; ZNF277; NOS3; POLN; SATB2; TMEM200A; ZNF385D; NOTCH4; POM121L12; SCAMP3; TNXB; ZNF394; NR1D2; POT1; SCN4A; TOX4; ZNF423; NRAS; PPARG; SEMA3D; TP53; ZNF445; NRG3; PPCS; SERPINA12; TP53INP1; ZNF511; NUTM1; PPP2R1B; SF3B1; TP63; ZNF536; OTOF; PRICKLE1; SGOL1; TPRG1; ZNF608; PAIP1; PRKAA2; SHOX2; TRHR; ZNF620; PCDH10; PRMT8; SIGLEC8; TRIM42; ZNF671; PCDH17; PSD2; SLAMF1; TRIM72; ZNF703; PCDH9; PSPH; SLC22A12; TRPC4; ZNF789; PCDHA1; PTCHD2; SLC32A1; TRPS1; ZSCAN22; PCDHA10; PTEN; SLC38A2; TSC1; ZZEF1; PCDHA11; PTPN22; SLC6A15; TSHZ3; PCDHA12; PTPN9; SLC6A20; TTC18; PCDHA2; PTPRK; SLTM; TTC19; PCDHA3; PTRF; SMARCA2; TUBGCP2; PCDHA4; PVRL4; SMARCA4; TXNIP; PCDHA5; PXDN; SNX22; UBL4A; PCDHA6; PXDNL; SNX32; UPF2; PCDHA7; PYDC1; SOX4; UVSSA; PCDHA8; RAB11FIP2; SOX5; VANGL1; PCDHA9; RAD51C; SPEG; VAT1; PCDHGA1; RARG; SPSB2; VCAM1; PCDHGA2; RB1; SPTAN1; VSX1; PCDHGA3; RBM10; STAG2; VWA8; PCDHGA4; RBM26; SUCNR1; VWF; or combinations thereof. In some embodiments, inferred tumor mutational burden (iTMB) is calculated by calculating the number of mutations or non-silent mutations in TMB-associated genes. In some embodiments, a subject is determined to have high inferred tumor mutational burden (high iTMB) if greater than 170 non-silent mutations from TMB-associated utDNA are detected per exome. In some embodiments, a subject is determined to have low inferred tumor mutational burden (low iTMB) if 170 or fewer non-silent mutations from TMB-associated utDNA are detected per exome. In some embodiments, if the subject is MRD-positive, the subject is predicted to be at higher risk for no pathologic complete response (pCR); or if the subject is MRD-negative, the subject is predicted to have pathologic complete response (pCR). In some embodiments, the subject is undergoing cancer treatment and a detected reduction in an MRD-associated utDNA during or after treatment is predictive of response to treatment. In some embodiments, the subject is undergoing cancer treatment and an increase in MRD-associated utDNA during or after treatment is predictive of a development of treatment resistance. In some embodiments, if the subject is utDNA MRD-negative, the subject is considered to have achieved a pathologic complete response (pCR) or is predicted to have a higher likelihood of pCR. In some embodiments, a utDNA MRD-negative subject is administered bladder-sparing treatment. In some embodiments, a utDNA MRD-negative subject is predicted to respond to bladder-sparing treatment. In some embodiments, a utDNA MRD-negative subject is determined to be a candidate for foregoing a radical cystectomy. In some embodiments, a utDNA MRD-negative subject is predicted to have long-term progression-free survival (PFS). In some embodiments, a utDNA MRD-positive subject is predicted to have significantly worse progression-free survival (PFS) compared to utDNA MRD-negative subjects. In some embodiments, a utDNA MRD-positive subject is predicted to benefit from surgical intervention, immune therapy, or both. In some embodiments, the immune therapy is adjuvant immunotherapy. In some embodiments, the immune therapy is targeted to the oncogenomic features of utDNA detected in utDNA MRD-positive subjects. In some embodiments, if a subject is MRD-positive the subject is predicted to have an absence of pathologic complete response (pCR). In some embodiments, if the subject is MRD-negative, the subject is predicted to have pathologic complete response (pCR). In some embodiments, if the subject is MRD-positive, the subject is predicted to not have pathologic complete response (pCR). In some embodiments, if the subject is utDNA MRD-negative the subject is predicted to have pathologic complete response (pCR) or disease-free survival (DFS). In some embodiments, if the subject is utDNA MRD-negative the subject is predicted to have pathologic complete response (pCR) after adjuvant therapy. In some embodiments, a utDNA MRD-positive subject is predicted to have worse progression-free survival compared to a utDNA MRD-negative subject. In some embodiments, a utDNA MRD-negative result in a subject undergoing cancer therapy (e.g., chemotherapy) is predictive of treatment response. In some embodiments, a utDNA MRD-positive result in a subject undergoing cancer therapy (e.g., chemotherapy) is predictive of treatment resistance. In some embodiments, if an ERCC2 nonsynonymous driver mutation is detected, the subject is predicted to be a responder to chemotherapy, optionally, neoadjuvant or adjuvant chemotherapy, optionally cisplatin-based chemotherapy. In some embodiments, a subject having less than 2.4% detected MRD-associated utDNA indicates (i) long-term survival and/or (ii) pathologic complete response (pCR) to neoadjuvant treatment and considered for bladder-sparing treatment. In some embodiments, a subject having more than 2.4% detected MRD-associated utDNA indicates the subject would be a poor candidate for neoadjuvant chemotherapy. In some embodiments, a subject having more than 2.4% detected MRD utDNA indicate the subject would be a candidate to proceed directly to partial or radical cystectomy. In some embodiments, if the subject is utDNA MRD-positive, the subject is determined to be at risk for significantly worse progression-free survival (PFS) for up 6 months after surgery, or at risk for significantly worse overall survival (OS). In some embodiments, if the subject is utDNA MRD-positive, the subject is determined to be at risk for early relapse within 200 days. In some embodiments, if the subject is utDNA MRD-positive, and the mutation has been associated with positive drug response in a cancer or solid tumor, the subject is administered the drug. In some embodiments, if the subject is utDNA MRD-positive, and the mutation has been not associated with positive drug response, the subject is administered other types of adjuvant therapy to ablate residual disease, optionally chemotherapy or radiation therapy. In some embodiments, if the subject is utDNA MRD-positive, and the mutation is in FGFR, the subject is administered erdafitinib. In some embodiments, if the subject is utDNA MRD-positive, and the mutation is in ERCC2, the subject is administered chemotherapy, optionally, neoadjuvant chemotherapy, optionally cisplatin-based chemotherapy. In some embodiments, if the subject is utDNA MRD-positive, and the mutation is in PIK3CA and TSC1, the subject is administered PI3K inhibition therapy or mTOR inhibition therapy. In some embodiments, the subject is administered a neoadjuvant therapy and modified or prolonged if the MRD utDNA increases or is MRD-positive after or during the standard course of treatment. In some embodiments, if the subject is utDNA MRD-negative, the subject is administered a bladder-sparing treatment. In some embodiments, if the subject is utDNA MRD-positive, the subject is administered adjuvant immunotherapy, optionally an adjuvant immune checkpoint blockade. In some embodiments, a subject having high inferred TMB (iTMB) is considered to be a candidate for treatment with early immune checkpoint blockade or early adjuvant immunotherapy, optionally, pre-surgical. In some embodiments, an MRD-positive subject having high iTMB is administered immunotherapy. In some embodiments, high iTMB subjects are predicted to have improved objective response rate (ORR), progression-free survival (PFS), and overall survival (OS) with response to immune checkpoint blockade. In some embodiments, a subject is utDNA MRD-positive and high iTMB is administered adjuvant immunotherapy, optionally an adjuvant immune checkpoint blockade. In some embodiments, the chemotherapy is selected from enfortumab-vedotin, methotrexate, vinblastine, doxorubicin, cisplatin, gemcitabine, erdafitinib, cabazitaxel, or combinations thereof. In some embodiments, the immunotherapy or immune adjuvant therapy is enfortumab, ipilimumab, nivolumab, pembrolizumab, or atezolizumab. In some embodiments, the neoadjuvant therapy can be modified to optimize candidacy for bladder-sparing treatment. In some embodiments, the method comprises treatment for a urinary tract-associated cancer, monitoring treatment, or monitoring progression of a urinary tract-associated cancer. In some embodiments, the method comprises monitoring of utDNA before, during, or after neoadjuvant chemotherapy to monitor treatment response versus the emergence of resistance, optimize the strength and regimentation of neoadjuvant treatment, and consideration and timing of radical cystectomy. In some embodiments, the method comprises treatment for a bladder cancer, such as localized bladder cancer, metastatic bladder cancer, muscle-invasive bladder cancer (MIBC), non-muscle invasive bladder cancer (NMIBC); urothelial carcinoma, papillary urothelial carcinoma, squamous cell carcinoma, or urothelial carcinoma with trophoblastic differentiation, including choriocarcinomatous differentiation; a urothelial carcinoma in the urinary tract; a genitourinary cancer, such as bladder cancer, kidney cancer, testicular cancer, urethral cancer, prostate cancer, penile cancer, ureteral cancer, urethral cancer, or adrenal cancer; a genitourinary cancer selected from adrenal cancer (e.g., adrenocortical carcinoma), bladder cancer (e.g., urothelial carcinoma, urachal cancer), kidney cancer (e.g., collecting duct carcinoma, renal cell carcinoma, rhabdoid tumor of the kidney, renal pelvic cancer, Wilms tumor (nephroblastoma)), penile cancer (e.g., urethral cancer), prostate cancer, or testicular cancer (germ cell tumor) (e.g., seminoma, non-seminoma, such as choriocarcinoma proud or teratoma); or kidney cancer, such as renal pelvis cancer, renal cell carcinoma, sarcomatoid (e.g., clear cell, papillary, chromophobe), medullary, collecting duct, urothelial carcinoma, sarcoma, Wilms tumor, lymphoma, oncocytoma, or angiomyolipoma. In some embodiments, the utDNA detection is performed prior to surgical intervention or after surgical intervention. In some embodiments, the utDNA detection is performed prior to, after, or during treatment, or has received, will be receiving, or is receiving neoadjuvant chemotherapy. In some embodiments, the surgical intervention is selected from: a partial or curative-intent radical cystectomy, such as a transurethral resection of bladder tumor tissue. In some embodiments, utDNA detection comprises detection of mutations in MRD-associated genes, the mutations selected from single nucleotide variants (SNVs). In some embodiments, utDNA detection comprises detection of mutations in MRD-associated genes, the mutations selected from genomic alterations selected from copy number variations, insertions/deletions, or fusions. In some embodiments, the method comprises detection of mutations in MRD-associated genes, the mutations selected from genomic alterations selected from copy number variations, insertions/deletions, or fusions. In some embodiments, the utDNA is detected by hybrid-capture cfDNA analysis. In some embodiments, the utDNA is detected using variant or mutational calling. In some embodiments, the utDNA is detected using variant calling of non-silent, duplex-supported mutations. In some embodiments, the utDNA level is determined by measuring the non-silent mutation level in variant allele fraction after removal of germline variants. In some embodiments, the utDNA is detected by uCAPP-Seq, a targeted next-generation sequencing method for detecting utDNA. In some embodiments, the method comprises detecting mutations with duplex support and does not require prior tumor mutational knowledge or prior sequencing of tumor tissue. In some embodiments, the utDNA detected is a mutation in an MRD-associated gene, a TMB-associated gene, or both. In some embodiments, the MRD-associated gene is selected from: ARID1A; CDKN1A; ERCC5; KMT2C; POLE; TP53; ASXL2; CDKN2A; FANCC; KMT2D; RB1; TSC1; ATM; CREBBP; FBXW7; KRAS; RHOA; TXNIP; ATR; ELF3; FGFR3; MDM2; RHOB; ZFP36L1; BRCA1; EP300; FOXA1; NF1; RXRA; BRCA2; ERBB2; FOXQ1; NFE2L2; SPTAN1; BTG2; ERBB3; HRAS; PAIP1; STAG2; CCND1; ERBB4; KDM6A; PIK3CA; TBC1D12; CCND3; ERCC2; KLF5; PLEKHS1; TERT; or combinations thereof. In some embodiments, the TMB-associated gene is selected from: ABCA6; C16orf71; COLGALT2; ERBB3; HCG4P5; KCNH6; ABCC5; C1QTNF1; COPB2; ERBB4; HCN1; KCTD19; ACAA1; C3orf17; COPG1; ERCC2; HECW1; KDM3A; ACPP; C3orf20; CPD; ERCC5; HELZ; KDM6A; ACTL8; C3orf58; CPSF6; FAM71A; HERC1; KIAA0100; ADAMTS12; C3orf70; CRB1; FAM71F1; HEXIM1; KIAA0556; ADAMTS16; C9orf156; CREBBP; FANCC; HHIP; KIAA1045; ADRA1A; C9orf64; CRTC2; FANCM; HHLA2; KLF5; AHR; CACNA1A; CRYAB; FBXL7; HIST1H1B; KLF6; AKT1; CACNA2D4; CSN3; FBXO42; HIST1H1C; KMT2C; ALAS1; CACNG4; CSPG5; FBXW7; HIST1H1E; KMT2D; ALDH16A1; CADM2; CTNNA2; FCRL3; HIST1H2AG; KRAS; ALYREF; CARD11; CTNNB1; FERD3L; HIST1H2AK; KRIT1; AMACR; CARD14; CTNND2; FETUB; HIST1H2BG; LAX1; AMIGO3; CBLB; CTTN; FFAR4; HIST1H2BH; LHFPL5; ANAPC11; CCDC129; CUBN; FGFR1; HIST1H2B1; LILRA2; ANKRD28; CCDC173; CUL1; FGFR2; HIST1H2BK; LILRB4; ANO4; CCDC36; CYP7A1; FGFR3; HIST1H3B; LIPH; APOA4; CCDC96; DACH1; FLRT2; HIST1H3F; LPAR6; AQP7; CCND1; DCAF12L2; FOXA1; HIST1H3H; LRGUK; ARHGEF16; CCND3; DCAF4L2; FOXA3; HIST1H3I; LRRC7; ARID1A; CCNE1; DDX42; FOXQ1; HIST1H4D; LRRK2; ARID4A; CCR5; DGCR8; FURIN; HIST1H4I; LRRN1; ASH1L; CD1 D; DGKG; FZD6; HIST1H4L; LRRTM1; ASIC2; CD5L; DHPS; GABRA1; HLA-A; LRTM2; ASXL2; CD86; DICER1; GABRB1; HLA-DQB2; MAGI1; ATM; CDC27; DIDO1; GABRG1; HNRNPA2B1; MAGI2; ATP10A; CDH1; DLC1; GALNT8; HOXA13; MAP1LC3A; ATP8B4; CDH10; DLEC1; GALR2; HOXA7; MAPK8; ATR; CDH6; DLGAP1; GAREM; HOXB1; MAPKAPK3; ATXN2L; CDK12; DLGAP5; GATA3; HOXB3; MARCHF1; AUTS2; CDK6; DNAH10; GCNT2; HOXB4; MB21D2; BCAS3; CDKN1A; DNAH11; GEMIN2; HRAS; MCL1; BCL2L1; CDKN2A; DNAH5; GHSR; HRH1; MCM7; BIRC6; CDKN2B; DNTT; GLI3; HSPB2; MDM2; BMS1; CEP63; DOPEY2; GMPPB; HSPB2-C11orf52; MED12; BPIFB4; CEP89; DPYS; GNA13; HTR1A; MED13; BRAF; CFL1; DSC1; GNAT1; HTRA2; MET; BRCA1; CHD2; DTX1; GNGT1; ID1; METTL3; BRCA2; CLASP2; E2F3; GOLGA1; IDH1; MGAT5B; BRINP3; CLCNKB; EEF1B2; GPC5; IDH2; MIOS; BRIP1; CLIP1; EFCAB5; GPC6; IKZF2; MIR1306; BRMS1; CLVS2; EGFR; GRIK3; IRX1; MIR4728; BTG2; CNKSR3; EIF6; GRM7; ITGA3; MLIP; C10orf90; CNTN1; ELF3; GSS; ITIH3; MMP25; C11orf16; CNTN6; EMILIN2; GTPBP8; JAK2; MMP9; C11orf65; CNTNAP1; EP300; GUCY1A3; KARS; MOCS1; C12orf43; COL11A2; EPHA3; GYS1; KCNA1; MPHOSPH8; C12orf66; COL1A2; EPHB4; H1FOO; KCNB2; MROH2B; C15orf52; COL6A3; ERBB2; HARB11; KCND2; MRPS22; MS4A3; PCDHGA5; RBM47; SUMF1; WAC; MTAP; PCDHGB1; RBP4; SYNE1; WDR64; MTOR; PCDHGB2; RECQL4; SYNE2; WNT7A; MYC; PCDP1; RECQL5; SYT10; XKR7; MYCL; PCF11; RHOA; SYT15; YAE1D1; MYF5; PCK1; RHOB; T; YAP1; MYH3; PCSK6; RHOU; TADA2B; YWHAZ; MYH7; PDE1C; RIPK4; TBC1D12; ZACN; MYO3A; PDE3A; RIT2; TBC1D16; ZBTB22; NAB2; PDE4DIP; RNASE3; TBKBP1; ZBTB45; NACA2; PEG3; RNF10; TCFL5; ZBTB7B; NANOS3; PGAP3; RNF111; TENM3; ZFC3H1; NCKAP5; PHACTR1; RNF123; TERT; ZFHX4; NCOR1; PHLDA3; RNF213; TFEC; ZFP36L1; NDUFAF6; PIGS; RNF216; TFP12; ZFP36L2; NEURL4; PIK3CA; RNF220; TGIF2LX; ZFYVE1; NF1; PKHD1; ROBO2; THAP2; ZFYVE9; NFE2L2; PKHD1L1; RTN4RL2; THSD7B; ZIC1; NKIRAS2; PLAUR; RUSC1; TIE1; ZIM2; NKTR; PLEKHA7; RXRA; TMCO2; ZIM3; NKX2-2; PLEKHS1; RYR2; TMCO4; ZNF263; NLRP8; POLDIP2; S1PR1; TMEM131; ZNF276; NOM1; POLE; SAMD4B; TMEM143; ZNF277; NOS3; POLN; SATB2; TMEM200A; ZNF385D; NOTCH4; POM121L12; SCAMP3; TNXB; ZNF394; NR1D2; POT1; SCN4A; TOX4; ZNF423; NRAS; PPARG; SEMA3D; TP53; ZNF445; NRG3; PPCS; SERPINA12; TP531NP1; ZNF511; NUTM1; PPP2R1B; SF3B1; TP63; ZNF536; OTOF; PRICKLE1; SGOL1; TPRG1; ZNF608; PAIP1; PRKAA2; SHOX2; TRHR; ZNF620; PCDH10; PRMT8; SIGLEC8; TRIM42; ZNF671; PCDH17; PSD2; SLAMF1; TRIM72; ZNF703; PCDH9; PSPH; SLC22A12; TRPC4; ZNF789; PCDHA1; PTCHD2; SLC32A1; TRPS1; ZSCAN22; PCDHA10; PTEN; SLC38A2; TSC1; ZZEF1; PCDHA11; PTPN22; SLC6A15; TSHZ3; PCDHA12; PTPN9; SLC6A20; TTC18; PCDHA2; PTPRK; SLTM; TTC19; PCDHA3; PTRF; SMARCA2; TUBGCP2; PCDHA4; PVRL4; SMARCA4; TXNIP; PCDHA5; PXDN; SNX22; UBL4A; PCDHA6; PXDNL; SNX32; UPF2; PCDHA7; PYDC1; SOX4; UVSSA; PCDHA8; RAB11FIP2; SOX5; VANGL1; PCDHA9; RAD51C; SPEG; VAT1; PCDHGA1; RARG; SPSB2; VCAM1; PCDHGA2; RB1; SPTAN1; VSX1; PCDHGA3; RBM10; STAG2; VWA8; PCDHGA4; RBM26; SUCNR1; VWF; or combinations thereof. In some embodiments, the method comprises the use of other emerging modalities in this regard, such as VI-RADS, an MRI-based staging criteria, to more definitively predict treatment response or clinical outcomes. In some embodiments, the method comprises urine cell-free DNA isolation and/or quantification comprising the steps of: (i) urine cfDNA was prepared from (e.g., 22-90 mL of) a urine sample with Q-Sepharose resin slurry (GE Healthcare) (e.g., at a ratio of 10 μL slurry per mL of urine) and mixed; (ii) after (i) (e.g., about 30 minutes), the urine/resin mixture was centrifuged (e.g., for about 10 minutes at 1800 g); (iii) the supernatant is discarded, and resin was washed twice (e.g., with 0.3M LiCl/10 mM sodium acetate (pH 5.5), 2 mL per 100 uL resin); (iv) the resin was transferred to a micro Bio-spin column (Bio-Rad, Hercules, CA), and the bound material was eluted by adding three separate (e.g., 670 uL) aliquots of (e.g., 2 M LiCl/10 mM) sodium acetate (e.g., pH 5.5); (v) next, the eluates were combined in (e.g., 70%) ethanol and passed over a column (e.g., QIAquick column); (vi) the column was washed (e.g., with 5 mL of 2 M LiCl in 70% ethanol followed by 5 mL of 75 mM potassium acetate (pH 8.0) in 80% ethanol); (vii) residual liquid was removed by centrifuging the columns (e.g., at 20,000 g for 3 minutes); (viii) bound DNA was eluted into (e.g., 50 μL of) nuclease-free water or (e.g., 10 mM) Tris (e.g., pH 8.5); and/or (ix) DNA concentrations were measured (e.g., using a Qubit dsDNA HS Assay Kit and Qubit 4.0 Fluorometer).

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1. Patient enrollment and sample collection for utDNA MRD analysis. Patients with localized bladder cancer who were candidates for radical cystectomy were enrolled onto this study, as were healthy adult donors. Biofluid samples were then collected for utDNA analysis as shown in the schema. MRD, minimal residual disease; pCR, pathologic complete response; uCAPP-Seq, urine Cancer Personalized Profiling by Deep Sequencing; utDNA, urine tumor DNA.

FIG. 2A-FIG. 2D. uCAPP-Seq application of MRD gene panel to urine samples. (A) Study schema. Urine and blood samples from 42 localized bladder cancer patients scheduled for radical cystectomy and 15 healthy adults were profiled by uCAPP-Seq. Samples from localized bladder cancer patients were collected preoperatively on the day of surgery. Sequencing libraries were prepared from urine cfDNA and peripheral blood germline DNA. utDNA analysis and results were primarily correlated with pathologic response in this study. (B) In silico application of the MRD panel, consisting of 49 consensus driver genes, to MIBC cases characterized by TCGA (n=409) and DFCI/MSKCC (n=50). A median of 5 mutations (silent and non-silent) per patient was detected in both datasets as indicated. (C) ROC curve classified pCR (n=16) from no pCR (n=26) patients by utDNA level, represented by the mutation with highest variant allele frequency (VAF). utDNA level derived from non-silent mutations (red curve) classified pathologic response significantly more accurately than silent mutations (blue curve). (D) Co-mutation plot showing non-silent driver mutations (with duplex support) detected in the urine of each patient with no pCR, patient with pCR, and healthy adult (bottom heatmap). utDNA level per variant is depicted within the heatmap, while the conglomerate level per subject is represented by the bar graph. Key patient characteristics are represented by the top heatmap. AUC, area under the curve; cfDNA, cell-free DNA; DFCI/MSKCC, Dana-Farber Cancer Institute and Memorial Sloan Kettering Cancer Center; MIBC, muscle-invasive bladder cancer; MRD, minimal residual disease; NGS, next generation sequencing; pCR, pathologic complete response; ROC, receiver operating characteristic; Sn, sensitivity; Sp, specificity; TCGA, The Cancer Genome Atlas; uCAPP-Seq, urine Cancer Personalized Profiling by Deep Sequencing; utDNA, urine tumor DNA; VAF, variant allele fraction.

FIG. 3A-FIG. 3D. utDNA MRD analysis in patients with localized bladder cancer. (A) Scatter plot of utDNA MRD levels (highest non-silent VAF per patient at preoperative time point) after square root transformation for 3 groups: healthy adults, patients with pCR, and patients with no pCR. The p-values were calculated using Mann-Whitney U test with a of 0.017 after Bonferroni correction. (B) ROC analysis classifying localized bladder cancer patients (either pCR or no pCR) versus healthy adults by utDNA level. AUC and Youden index-optimized sensitivity and specificity values are shown. (C) Stacked bar plots showing the proportions of each group with positive or negative utDNA MRD detection, determined using the optimal utDNA level threshold (VAF: 2.3%) that classified patients with no pCR from healthy adults. The p-values were calculated using Fisher exact test with a of 0.017 after Bonferroni correction. (D) Multivariate logistic regression predicting the absence of pCR. The heatmap shows logarithm of the OR for each variable in the logistic regression. Each bar shows a negative logarithm of the p-value, with the dashed line denoting a of 0.05. AUC, area under the curve; MRD, minimal residual disease; OR, odds ratio; pCR, pathologic complete response; ROC, receiver operating characteristic; Sn, sensitivity; Sp, specificity; Sqrt, square root; utDNA, urine tumor DNA; VAF, variant allele fraction; Y/N, yes/no.

FIG. 4A-FIG. 4D. Preoperative utDNA MRD as an early prognostic biomarker in localized bladder cancer patients. (A) Kaplan-Meier analysis of PFS stratified by preoperative utDNA MRD detection and (B) pathologic response after radical cystectomy. utDNA MRD (+) patients had significantly worse PFS than utDNA MRD (−) patients (HR=7.4; 95% CI: 1.4-38.9; p=0.02). Patients with no pCR also had significantly worse PFS than patients with pCR (HR=5.5; 95% CI: 1.1-28.1; p=0.04). HRs and p-values were calculated using the Mantel-Haenszel and Mantel-Cox tests, respectively. (C) Serial urine cfDNA analysis for MIBC patient BC-1050. Imaging after neoadjuvant chemotherapy was equivocal with residual bladder wall thickening. utDNA levels increased during neoadjuvant chemotherapy with a COSMIC- and OncoKB-annotated nonsynonymous ERCC2 mutation having the highest VAF. utDNA levels then decreased and became undetectable at the preoperative MRD timepoint, concordant with pCR on surgical pathology. The patient remained disease free on long-term follow-up. (D) Serial urine cfDNA analysis for MIBC patient BC-1045. Imaging after neoadjuvant chemotherapy noted decreased but persistent bladder wall thickening. utDNA levels decreased during neoadjuvant chemotherapy, with a KMT2D stop-gain mutation having the highest VAF. utDNA levels then increased at the preoperative MRD time point with evidence of 2 new emergent nonsynonymous mutations in ERBB2 and ARID1A, not present earlier. Consistent with this positive utDNA MRD result, the patient had pathological nodal involvement despite achieving a pCR in the urothelium, and, less than a year later, developed progressive disease that prompted the start of treatment with atezolizumab. cfDNA, cell-free DNA; CI, confidence interval; COSMIC, Catalogue of Somatic Mutations in Cancer; HR, hazard ratio; MIBC, muscle-invasive bladder cancer; MRD, minimal residual disease; pCR, pathologic complete response; PFS, progression-free survival; utDNA, urine tumor DNA; VAF, variant allele fraction.

FIG. 5A-FIG. 5D. utDNA MRD as a biomarker for adjuvant treatment personalization. (A) Comparison of non-silent mutational load detected by whole exome sequencing and uCAPP-Seq for MIBC cases in TCGA (n=409), using an expanded gene panel for assessing TMB (referred to as the TMB panel; see e.g., TABLE 9). Linear regression was performed (Pearson's r=0.84; equation shown) to interpolate exome-wide TMB based on the number of non-silent mutations detected by uCAPP-Seq. (B) Scatter plot depicting inferred TMB among patients with positive utDNA MRD in the cohort with the median indicated by a horizontal black line. (C) Potential adjuvant treatment strategies for utDNA MRD (+) patients based on urine-inferred TMB. (D) Vignette depicting MIBC patient BC-1171 who was utDNA MRD (+) at the preoperative time point, concordant with no pCR on surgical pathology with evidence of locally advanced pT3a disease (AJCC 8). Six non-silent mutations were detected in preoperative urine from this patient as indicated. Urine-inferred TMB was elevated at 204. This patient was randomized to pembrolizumab on a clinical trial and has shown no evidence of disease on long-term follow-up, consistent with the prediction for a patient with high inferred TMB. AJCC 8, American Joint Committee on Cancer 8^{th} edition; MIBC, muscle-invasive bladder cancer; MRD, minimal residual disease; pCR, pathologic complete response; TCGA, The Cancer Genome Atlas; TMB, tumor mutational burden; TURBT, transurethral resection of bladder tumor; tx, treatment; uCAPP-Seq, urine Cancer Personalized Profiling by Deep Sequencing; utDNA, urine tumor DNA.

FIG. 6. EDTA prevents urine cfDNA degradation. Technical experiment to evaluate the stability of urine cfDNA with and without the addition of EDTA. Pooled urine from a healthy volunteer was stored for 7 days with versus without 10 mM EDTA at room temperature. Urine cfDNA was extracted at days 0, 1, 3, and 7 after commencing storage. The effect of EDTA on urine cfDNA degradation was evaluated based on the percentage of DNA in the 70-450 bp size range on Bioanalyzer electropherogram. The data presented here are mean±SEM of 2 replicates per time point. The p-value was calculated by unpaired Student t test at the last time point. bp, base pair; cfDNA, cell-free DNA; SEM, standard error of the mean.

FIG. 7. Schema for comparing in vitro size selection against acoustic fragmentation of urine cfDNA. Representative electropherograms are also shown, all from the same MIBC patient's urine sample. bp, base pair; cfDNA, cell-free DNA; MIBC, muscle-invasive bladder cancer; mM, millimolar; uCAPP-Seq, urine Cancer Personalized Profiling by Deep Sequencing.

FIG. 8A-FIG. 8C. Comparison of acoustic whole urine cfDNA fragmentation to in vitro size selection of 70-450 bp cfDNA fragments. (A) Median deduplicated sequencing depths, (B) Number of non-silent mutations, (C) Mutant allele fraction levels in 4 MIBC urine cfDNA samples that underwent in vitro size selection or acoustic DNA fragmentation (see e.g., FIG. 7). Data values are depicted as points, column heights represent averages, and error bars represent standard error of the mean. VAF comparisons in (C) are also shown with connecting lines. The p values were calculated using the paired Student t test method. bp, base pair; cfDNA, cell-free DNA; MIBC, muscle-invasive bladder cancer; VAF, variant allele fraction.

FIG. 9. ROC analysis classifying pCR versus no pCR with different variant calling criteria. The red curve shows variant calling of non-silent, duplex-supported mutations in the MRD panel. The blue curve shows variant calling of non-silent, duplex-supported mutations in the TMB panel. The green curve shows variant calling of all mutations (silent or non-silent) with duplex support in the TMB panel. The AUC, as well as the optimal sensitivity and specificity values corresponding to Youden J statistic, is shown. AUC, area under the curve; MRD, minimal residual disease; pCR, pathologic complete response; ROC, receiver operating characteristic; Sn, sensitivity; Sp, specificity; TMB, tumor mutational burden.

FIG. 10A-FIG. 10D. utDNA MRD analysis in MIBC patients treated with neoadjuvant chemotherapy. (A) ROC analysis classifying MIBC patients (either pCR or no pCR) who received neoadjuvant chemotherapy before radical cystectomy based on non-silent versus silent mutations detected in preoperative urine cfDNA. The AUC, as well as the optimal sensitivity and specificity values corresponding to Youden J statistic, is shown. (B) Scatter plot of utDNA levels (highest nonsilent VAF per patient at the preoperative time point) after square root transformation in patients who achieved pCR versus those with no pCR following neoadjuvant chemotherapy. The p-value was calculated using Mann-Whitney U test. (C) Stacked bar plots showing proportions of each group with positive or negative utDNA MRD detection after neoadjuvant chemotherapy, determined by the optimal utDNA threshold (VAF: 2.3%) that classified patients with no pCR from healthy adults in the full patient cohort. The p-value was calculated using Fisher exact test. (D) Scatter plot of utDNA levels after square root transformation in patients who received neoadjuvant chemotherapy, grouped by pathologic stage. Column heights depict mean values, and error bars represent the standard error of the mean. The Kruskal-Wallis H statistic and p-value are shown in the upper left. There were no patients with ypT1 disease. AUC, area under the curve; cfDNA, cell-free DNA; MIBC, muscle-invasive bladder cancer; MRD, minimal residual disease; pCR, pathologic complete response; ROC, receiver operating characteristic; Sn, sensitivity; Sp, specificity; Sqrt, square root; utDNA, urine tumor DNA; VAF, variant allele fraction.

FIG. 11A-FIG. 11B. Correlation of utDNA MRD detection with pathologic nonresponse in 2 MIBC patients. (A) Clinical vignette of MIBC patient BC-1071 who was utDNA MRD (+) at the preoperative time point with a utDNA level of 10.1%, which was concordant with no pCR on surgical pathology. The patient progressed 160 days later and died shortly thereafter. (B) Clinical vignette of MIBC patient BC-1206 who was utDNA MRD (+) at the preoperative time point with a utDNA level of 2.8%, which was concordant with no pCR on surgical pathology. The patient progressed at 46 days after radical cystectomy, received chemoradiation to treat the recurrence, but, nonetheless, died at 164 days. MIBC, muscle-invasive bladder cancer; MRD, minimal residual disease; pCR, pathologic complete response; TURBT, transurethral resection of bladder tumor; utDNA, urine tumor DNA.

FIG. 12A-FIG. 12B. Correlation of utDNA MRD detection and pathologic response with OS. OS analysis according to utDNA MRD detection at the preoperative time point (A) and pathologic response assessed in the radical cystectomy specimen (B). HRs and p-values were calculated using the Mantel-Haenszel and Mantel-Cox methods, respectively. CI, confidence interval; HR, hazard ratio; MRD, minimal residual disease; OS, overall survival; pCR, pathologic complete response, utDNA, urine tumor DNA.

### DETAILED DESCRIPTION

The present disclosure is based, at least in part, on the discovery that tumor DNA collected from urine samples can be used to predict minimal residual disease. As shown herein, a gene panel predicts minimal residual disease and tumor mutational burden and can be used to guide treatment for bladder cancer.

The present disclosure provides for urine liquid biopsy gene panels to detect minimal residual disease and infer tumor mutational burden. Described herein are 2 gene panels—a minimal residual disease (MRD) panel and a tumor mutational burden (TMB) panel—for urine liquid biopsy residual disease detection and precision guidance of adjuvant treatment including immunotherapy.

The disclosed technology solves the problem of noninvasively detecting residual disease after neoadjuvant treatment for muscle-invasive bladder cancer. There is no reliable way of doing this, and as a result, some patients end up getting radical surgery that may have been unnecessary (based on no evidence of disease in the surgical sample).

A second problem the disclosed technology solves is to precisely determine what adjuvant treatment to deliver. Here, it is shown that the disclosed method could help select between immunotherapy, targeted systemic therapy, and chemotherapy based on the oncogenomic profile detected in urine using the custom gene panels.

Pathologic complete response (pCR) is the lack of all signs of cancer in tissue samples removed during surgery or biopsy after treatment with radiation or chemotherapy. To find out if there is a pathologic complete response, a pathologist checks the tissue samples under a microscope to see if there are still cancer cells left after the anticancer treatment. Knowing if the cancer is in pathologic complete response may help show how well treatment is working or if the cancer will come back (also called pathologic complete remission).

As described herein, mutations in MRD-associated utDNA and TMB-associated utDNA, include measuring single nucleotide variants (SNVs). Sometimes SNVs can be single nucleotide polymorphisms (SNPs), but SNVs and SNPs are not interchangeable. To qualify as an SNP, the variant must be present in at least 1% of the population.

Urinary Tract-Associated Cancer

Methods and compositions as described herein can be used for the prevention, treatment, or slowing the progression of cancer or tumor growth. For example, the cancer can be a cancer associated with the urinary tract.

The methods described here can be useful for any urothelial carcinoma (also known as transitional cell carcinoma (TCC)) at any part of the urinary tract. For example, the urothelial carcinoma can be all types of bladder cancers, genitourinary cancers, cancers of the urinary tract, kidney cancers, ureteral cancers, or renal pelvis cancers. Examples of kidney cancers can be renal cell carcinoma (e.g., kidney cancer types: sarcomatoid (e.g., clear cell, papillary, chromophobe), medullary, collecting duct, urothelial carcinoma, sarcoma, Wilms tumor, lymphoma, oncocytoma, angiomyolipoma).

Genitourinary cancer can be bladder cancer, kidney cancer, testicular cancer, urethral cancer, prostate cancer, penile cancer, urethral cancer, or adrenal cancer. Genitourinary cancer types can include adrenal cancer (e.g., adrenocortical carcinoma), bladder cancer (e.g., urothelial carcinoma, urachal cancer), kidney cancer (e.g., collecting duct carcinoma, renal cell carcinoma, rhabdoid tumor of the kidney, Renal pelvic cancer (ureteral cancer), Wilms tumor (nephroblastoma)), penile cancer (e.g., urethral cancer), prostate cancer, testicular cancer (germ cell tumor) (e.g., seminoma, non-seminoma, such as choriocarcinoma proud or teratoma).

As another example, the cancer associated with the urinary tract can be bladder cancer, kidney cancer, ureter cancer, urothelial carcinoma, squamous cell carcinoma, adenocarcinoma papillary carcinomas, flat carcinomas, localized bladder cancer, metastatic bladder cancer, muscle-invasive bladder cancer (MIBC), non-muscle invasive bladder cancer (NMIBC), or urothelial carcinoma with trophoblastic differentiation, including choriocarcinomatous differentiation.

Cancer Treatment

Described herein are methods to treat cancer and determine if a subject would be responsive to a therapy, surgery, adjuvant therapy, or neoadjuvant therapy.

Neoadjuvant is generally delivered before surgery with the goal of shrinking a tumor or stopping the spread of cancer to make surgery less invasive and more effective. Adjuvant therapy can be administered after surgery to kill any remaining cancer cells with the goal of reducing the chances of recurrence.

Chemotherapy

The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Standard of care for bladder cancers currently can include nivolumab, pembrolizumab, atezolizumab, enfortumab-vedotin, erdafitinib, methotrexate, vinblastine, doxorubicin, cisplatin, and gemcitabine (and combinations thereof). Examples of other chemotherapeutic agents can include alkylating agents such as thiotepa and cyclophosphamide; alkyl sulfonates such as busulfan, improsulfan and piposulfan; aziridines such as benzodopa, carboquone, meturedopa, and uredopa; ethylenimines and methylamelamines such as altretamine, triethylenemelamine, trietylenephosphoramide, triethiylenethiophosphoramide and trimethylolomelamine; acetogenins (such as bullatacin and bullatacinone); a camptothecin (such as the synthetic analogue topotecan); bryostatin; callystatin; CC-1065 (such as its adozelesin, carzelesin and bizelesin synthetic analogues); cryptophycins (particularly cryptophycin 1 and cryptophycin 8); dolastatin; duocarmycin (such as the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin γ1 and calicheamicin ω1; dynemicin, such as dynemicin A; uncialamycin and derivatives thereof; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-I-norleucine, doxorubicin (e.g., morpholino-doxorubicin, cyanomorpholino-doxorubicin, 2-pyrrolino-doxorubicin, or deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, or zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichloro-triethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and docetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; mitoxantrone; teniposide; edatrexate; daunomycin; aminopterin; capecitabine; ibandronate; irinotecan (e.g., CPT-11); topoisomerase inhibitor RFS 2000; difluorometlhylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosourea, dactinomycin, daunorubicin, doxorubicin, bleomycin, plicomycin, mitomycin, etoposide (VP16), tamoxifen, raloxifene, estrogen receptor binding agents, paclitaxel, docetaxel, gemcitabine, vinorelbine, farnesyl-protein transferase inhibitors, transplatinum, 5-fluorouracil, vincristine, vinblastine, or methotrexate or pharmaceutically acceptable salts, acids or derivatives of any of the above. Other examples of chemotherapeutic agents can be Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alkeran (Melphalan Hydrochloride), Alkeran (Melphalan), Alimta (Pemetrexed Disodium), Aloxi (Palonosetron Hydrochloride), Ambochlorin/Amboclorin (Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar (Irinotecan Hydrochloride), Capecitabine, CAPOX, Carac (Fluorouracil-Topical), Carboplatin, Carboplatin-Taxol, Carfilzomib, Carmubris (Carmustine), Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, Chlorambucil-prednisone, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Efudex (Fluorouracil-Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista (Raloxifene Hydrochloride), Evomela (Melphalan Hydrochloride), Exemestane, 5-FU (Fluorouracil Injection), 5-FU (Fluorouracil-Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil-Topical), Fluorouracil Injection, Fluorouracil-Topical, Flutamide, Folex (Methotrexate), Folex PFS (Methotrexate), FOLFIRI, FOLFIRI-bevacizumab, FOLFIRI-Cetuximab, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, Gemcitabine-Cisplatin, Gemcitabine-Oxaliplatin, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Ifex (Ifosfamide), Ifosfamide, Ifosfamidum (Ifosfamide), IL-2 (Aldesleukin), Imatinib Mesylate, Imbruvica (Ibrutinib), Imiquimod, Imlygic (Talimogene Laherparepvec), Inlyta (Axitinib), Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituximab, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq (Atezolizumab), Temodar (Temozolomide), Temozolomide, Temsirolimus, Thalidomide, Thalomid (Thalidomide), Thioguanine, Thiotepa, Tolak (Fluorouracil-Topical), Topotecan Hydrochloride, Toremifene, Torisel (Temsirolimus), Tositumomab and Iodine 1131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VelP, Velban (Vinblastine Sulfate), Velcade (Bortezomib), Velsar (Vinblastine Sulfate), Vemurafenib, Venclexta (Venetoclax), Venetoclax, Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zelboraf (Vemurafenib), Zevalin (lbritumomab Tiuxetan), Zinecard (Dexrazoxane Hydrochloride), Ziv-Aflibercept, Zoladex (Goserelin Acetate), Zoledronic Acid, Zolinza (Vorinostat), Zometa (Zoledronic Acid), Zydelig (Idelalisib), Zykadia (Ceritinib), or Zytiga (Abiraterone Acetate) or pharmaceutically acceptable salts, acids or derivatives of any of the above.

Immunotherapy

As described herein, the provided methods allow for the identification of subjects who can benefit from immunotherapies. Immunotherapies are a new generation of cancer therapy that has revolutionized the treatment of otherwise terminal cancers, often achieving durable, sustained remission in cancers that were otherwise thought to be refractory to standard first- and second-line therapies. Thousands of patients annually are now treated with these life-saving therapies.

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

Examples of immunotherapy can be immune effector cell (IEC) therapy (e.g., CAR T, mesenchymal stem cells) or T cell engaging therapy (e.g., CD19-specific T cell engager, such as blinatumomab, T cell engaging monoclonal antibody, bispecific T cell engager (BiTE) therapy).

In some embodiments, the provided methods are used before, after, or in concurrence with any form of bispecific monoclonal antibody (BsMAb) therapy. For example, the BsMAb therapy can be any one or more of the currently FDA-approved BsMAb therapies, such as blinatumomab, emicizumab, or amivantamab.

In one aspect of immunotherapy, the tumor cell must bear some marker that is amenable to targeting, i.e., is not present on the majority of other cells. Many tumor markers exist and any of these may be suitable for targeting in the context of the present disclosure. Markers described herein are those in TABLE 3 and TABLE 9. Other common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, γ-IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds may be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides, et al., 1998), cytokine therapy, e.g., interferons α, β, and γ; IL-1, GM-CSF, TNF (Bukowski, et al., 1998; Davidson, et al., 1998; Hellstrand, et al., 1998) gene therapy, e.g., TNF, IL-1, IL-2, p53 (Qin et al., 1998; Austin-Ward and Villaseca, 1998; U.S. Pat. Nos. 5,830,880 and 5,846,945), and monoclonal antibodies, e.g., anti-ganglioside GM2, anti-HER-2, anti-p185 (Pietras, et al., 1998; Hanibuchi, et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton, et al., 1992; Mitchell, et al., 1990; Mitchell, et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg, et al., 1988; 1989).

In some embodiments, the immunotherapy in accordance with the present disclosure is CAR T cell therapy (e.g., CD19-specific chimeric antigen receptor T (CAR-T)). Generally, CAR T cell therapy refers to any type of immunotherapy in which a subject's T cells are genetically modified to express chimeric antigen receptors. These chimeric antigen receptors allow the T cells to more effectively recognize and subsequently destroy cancer cells. Typically, T cells are first harvested from a subject, genetically altered to express a CAR targeting an antigen of interest (e.g., an antigen expressed on the surface of a tumor or cancer cell), and then infused back into the subject. Once infused into the subject, CAR T cells bind to the target antigen and are activated, allowing them to proliferate and become cytotoxic.

Checkpoint Immunotherapy

In some embodiments, the present disclosure provides for a prediction of checkpoint immunotherapy response. An important function of the immune system is its ability to tell between normal cells in the body and those it sees as “foreign.” This lets the immune system attack the foreign cells while leaving the normal cells alone. To do this, it uses “checkpoints.” Immune checkpoints are molecules on certain immune cells that need to be activated (or inactivated) to start an immune response.

Cancer cells can find ways to use these checkpoints to avoid being attacked by the immune system. But drugs that target these checkpoints hold a lot of promise as a cancer treatment. These drugs are called checkpoint inhibitors. Checkpoint inhibitors used to treat cancer do not work directly on the tumor at all. They only take the brakes off an immune response that has begun but has not yet been working at its full force.

Checkpoint immunotherapy has been extensively shown to unleash T cell effector functions to control tumors in many cancer patients. However, tumor cells can evade immunological elimination by recruiting myeloid cells that induce an immunosuppressive state. Recent high dimensional profiling studies have shown that tumor-infiltrating myeloid cells are considerably heterogeneous, and may include both immunostimulatory and immunosuppressive subsets, although they do not fit the M1/M2 paradigm. Thus, depletion of suppressive myeloid cells from tumors, blockade of their functions, or induction of myeloid cells with immunostimulatory properties may provide important approaches for improving immunotherapy strategies, perhaps in synergy with checkpoint blockade.

Any immune checkpoint inhibitor known in the art can be used. For example, a PD-1 inhibitor can be used. These drugs are typically administered IV (intravenously). PD-1 is a checkpoint protein on immune cells called T cells. It normally acts as a type of “off switch” that helps keep the T cells from attacking other cells in the body. It does this when it attaches to PD-L1, a protein on some normal (and cancer) cells. When PD-1 binds to PD-L1, it tells the T cell to leave the other cell alone. Some cancer cells have large amounts of PD-L1, which helps them hide from an immune attack.

Monoclonal antibodies that target either PD-1 or PD-L1 can block this binding and boost the immune response against cancer cells. These drugs have shown a great deal of promise in treating certain cancers.

Examples of drugs that target PD-1 can include: Pembrolizumab (Keytruda), Nivolumab (Opdivo), Atezolizumab, or Cemiplimab (Libtayo). These drugs have been shown to be helpful in treating several types of cancer, and new cancer types are being added as more studies show these drugs to be effective.

As another example, a PD-L1 inhibitor can be used. Examples of drugs that target PD-L1 can include: Atezolizumab (Tecentriq), Avelumab (Bavencio), or Durvalumab (Imfinzi). These drugs have also been shown to be helpful in treating different types of cancer, and are being studied for use against others.

CTLA-4 is another protein on some T cells that acts as a type of “off switch” to keep the immune system in check. For example, Ipilimumab (Yervoy) is a monoclonal antibody that attaches to CTLA-4 and reduces or blocks its function. This can boost the body's immune response against cancer cells. This drug can be used to treat melanoma of the skin and other cancers.

Radiotherapy

In some embodiments, the methods described herein can provide evidence that a radiotherapy may be administered to a patient. Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter can repair themselves and function properly.

Radiation therapy used according to the present disclosure may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors induce a broad range of damage on DNA, on the precursors of DNA, on the replication and repair of DNA, and on the assembly and maintenance of chromosomes. Dosage ranges for X-rays range from daily doses of 12.9 to 51.6 mC/kg for prolonged periods of time (3 to 4 wk), to single doses of 0.516 to 1.55 C/kg. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer or tumor. This ensures that a higher radiation dose is given to the tumor. Healthy surrounding cells and nearby structures receive a lower dose of radiation, so the possibility of side effects is reduced. A device called a multi-leaf collimator has been developed and may be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

Therapeutic Methods

Also provided is a process of treating, preventing, or reversing bladder cancer in a subject in need of a bladder removal or administration of a therapeutically effective amount of an anticancer agent or treatment.

Methods described herein are generally performed on a subject in need thereof. A subject in need of the therapeutic methods described herein can be a subject having, diagnosed with, suspected of having, or at risk for developing bladder cancer. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of an anticancer therapy can occur as a single event or over a time course of treatment. For example, an anticancer therapy can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to or before, concurrent with, or after conventional treatment modalities for cancer.

Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

Molecular Engineering

The following definitions and methods are provided to better define the present disclosure and to guide those of ordinary skill in the art in the practice of the present disclosure. Unless otherwise noted, terms are to be understood according to conventional usage by those of ordinary skill in the relevant art.

The terms “heterologous DNA sequence”, “exogenous DNA segment”, or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A. For example, the percent identity can be at least 80% or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Substitution refers to the replacement of one amino acid with another amino acid in a protein or the replacement of one nucleotide with another in DNA or RNA. Insertion refers to the insertion of one or more amino acids in a protein or the insertion of one or more nucleotides with another in DNA or RNA. Deletion refers to the deletion of one or more amino acids in a protein or the deletion of one or more nucleotides with another in DNA or RNA. Generally, substitutions, insertions, or deletions can be made at any position so long as the required activity is retained.

So-called conservative exchanges can be carried out in which the amino acid which is replaced has a similar property as the original amino acid, for example, the exchange of Glu by Asp, Gln by Asn, Val by lie, Leu by lie, and Ser by Thr. For example, amino acids with similar properties can be Aliphatic amino acids (e.g., Glycine, Alanine, Valine, Leucine, Isoleucine); hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41 (fraction G/C content)−0.63(% formamide)−(600/I). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids that may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to a gene chip, EDTA, a centrifuge, resin slurry, LiCl, sodium acetate, micro spin column, micro column, potassium acetate, ethanol, nuclease free water, Tris, dsDNA assay kit, or a fluorometer. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit, instead, a user may be directed to an Internet web site specified by the manufacturer or distributor of the kit.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects or a wild-type subject or sample. A control sample or a reference sample can also be a sample with a known amount of a detectable compound or a spiked sample.

The methods and algorithms of the invention may be enclosed in a controller or processor. Furthermore, methods and algorithms of the present invention, can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer program include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Urine Tumor DNA Detection of Minimal Residual Disease in Muscle-Invasive Bladder Cancer Treated with Curative-Intent Radical Cystectomy

This example describes the use of detecting urine tumor DNA to offer bladder-sparing treatment to subjects that may not require a radical cystectomy.

Abstract

Background: The standard of care treatment for muscle-invasive bladder cancer (MIBC) is radical cystectomy, which is typically preceded by neoadjuvant chemotherapy. However, the inability to assess minimal residual disease (MRD) noninvasively limits the ability to offer bladder-sparing treatment. Herein is described the development of a liquid biopsy solution via urine tumor DNA (utDNA) analysis.

Methods and findings: Urine Cancer Personalized Profiling by Deep Sequencing (uCAPP-Seq), a targeted next-generation sequencing (NGS) method for detecting utDNA, was applied to urine cell-free DNA (cfDNA) samples acquired between April 2019 and November 2020 on the day of curative-intent radical cystectomy from 42 patients with localized bladder cancer. The average age of patients was 69 years (range: 50 to 86), of whom 76% ( 32/42) were male, 64% ( 27/42) were smokers, and 76% ( 32/42) had a confirmed diagnosis of MIBC. Among MIBC patients, 59% ( 19/32) received neoadjuvant chemotherapy. utDNA variant calling was performed noninvasively without prior sequencing of tumor tissue. The overall utDNA level for each patient was represented by the non-silent mutation with the highest variant allele fraction after removing germline variants. Urine was similarly analyzed from 15 healthy adults. utDNA analysis revealed a median utDNA level of 0% in healthy adults and 2.4% in bladder cancer patients. When patients were classified as those who had residual disease detected in their surgical sample (n=26) compared to those who achieved a pathologic complete response (pCR; n=16), median utDNA levels were 4.3% vs. 0%, respectively (p=0.002). Using an optimal utDNA threshold to define MRD detection, positive utDNA MRD detection was highly correlated with the absence of pCR (p<0.001) with a sensitivity of 81% and specificity of 81%. Leave-one-out cross-validation applied to the prediction of pathologic response based on utDNA MRD detection in the cohort yielded a highly significant accuracy of 81% (p=0.007). Moreover, utDNA MRD-positive patients exhibited significantly worse progression-free survival (PFS; HR=7.4; 95% CI: 1.4-38.9; p=0.02) compared to utDNA MRD-negative patients. Concordance between urine- and tumor-derived mutations, determined in 5 MIBC patients, was 85%. Tumor mutational burden (TMB) in utDNA MRD-positive patients was inferred from the number of non-silent mutations detected in urine cfDNA by applying a linear relationship derived from The Cancer Genome Atlas (TCGA) whole exome sequencing of 409 MIBC tumors. About 58% of these patients with high inferred TMB might have been candidates for treatment with early immune checkpoint blockade. Study limitations included an analysis restricted only to single-nucleotide variants (SNVs), survival differences diminished by surgery, and a low number of DNA damage response (DRR) mutations detected after neoadjuvant chemotherapy at the MRD time point.

Conclusions: utDNA MRD detection prior to curative-intent radical cystectomy for bladder cancer correlated significantly with pathologic response, which may help select patients for bladder-sparing treatment. utDNA MRD detection also correlated significantly with PFS. Furthermore, utDNA can be used to noninvasively infer TMB, which could facilitate personalized immunotherapy for bladder cancer in the future.

Introduction

In the United States of America, bladder cancer is the sixth most commonly diagnosed cancer overall and the fourth most commonly diagnosed malignancy in men. Clinical management is largely dependent on the extent to which the tumor penetrates the bladder wall at time of presentation. Approximately 70% of patients initially present with non-muscle-invasive bladder cancer (NMIBC). These patients are typically treated with transurethral resection of bladder tumor alone or in conjunction with intravesical therapy. While 5-year survival rates for NMIBC are favorable, patients carry a high risk of recurrence or progression to more advanced stages of disease. Therefore, these patients require frequent monitoring with invasive cystoscopic examinations. As a result, bladder cancer has been found to have the highest lifetime cost per patient of any malignancy.

Approximately 25% of bladder cancer patients present with organ-confined muscle-invasive bladder cancer (MIBC), characterized by tumor that extends to the underlying detrusor muscle. The standard of care treatment recommendation for both MIBC and recurrent/progressive NMIBC after appropriate intravesical therapy is radical cystectomy with urinary diversion, a significant surgical procedure with a major impact on quality of life. For MIBC patients, this is often preceded by cisplatin-based neoadjuvant chemotherapy. Despite these aggressive measures, the overall 5-year survival for MIBC is still only about 50%, and the recurrence risk is substantial.

Pathologic response is a strong prognostic indicator of long-term survival after radical cystectomy. Based on evaluation of the radical cystectomy specimen, approximately 35% of patients will achieve a pathologic complete response (pCR) following neoadjuvant treatment. Retrospective data support the idea that some patients who achieve an excellent response to neoadjuvant treatment may be able to safely forego radical cystectomy. If patients likely to have achieved a pCR could be better identified prospectively, this could support personalized care, enabling some patients to avoid the life-altering morbidity of urinary diversion and potential mortality from operative complications.

Cystoscopy, while considered invasive and uncomfortable, can be used to evaluate tumor response to neoadjuvant chemotherapy or chemoradiation. However, cystoscopy prior to radical cystectomy frequently under-stages disease burden, creating a major barrier to testing and implementing personalized bladder-sparing treatment paradigms. Furthermore, patients managed with bladder-sparing treatment remain at significant risk for recurrence, thus requiring frequent monitoring with invasive cystoscopy. Therefore, it is critical that the standard of care is enhanced to enable improved accuracy and reduced morbidity when assessing and monitoring localized bladder cancer.

Plasma- and urine-based biomarkers present a promising opportunity for noninvasive management of localized bladder cancer. Cancer Personalized Profiling by Deep Sequencing (CAPP-Seq) with integrated digital error suppression (iDES) is an ultrasensitive method of high-throughput sequencing used to detect circulating tumor DNA (ctDNA). In recent years, the utility of CAPP-Seq with iDES in the detection of minimal residual disease (MRD) has been validated in a variety of tumor types. Urine tumor DNA (utDNA) analysis with an adapted version of CAPP-Seq, called urine Cancer Personalized Profiling by Deep Sequencing (uCAPP-Seq), was recently shown to be a highly sensitive urine-based approach for monitoring NMIBC patients treated conservatively with modalities other than radical cystectomy.

Herein, uCAPP-Seq is validated by applying it to urine collected from a largely MIBC cohort with every patient treated with curative-intent radical cystectomy. It was determined whether utDNA variant calling without prior sequencing of tumor tissue using urine samples collected just prior to radical cystectomy can predict pathologic staging and differences in survival outcomes, namely progression-free survival (PFS) and overall survival (OS). Furthermore, the hypothesis that utDNA can be used to infer tumor mutational burden (TMB) and identify actionable mutations, which could potentially inform personalized adjuvant treatment including immune checkpoint blockade and targeted systemic therapy for utDNA MRD-positive patients, was addressed.

Results

Cohort Characteristics

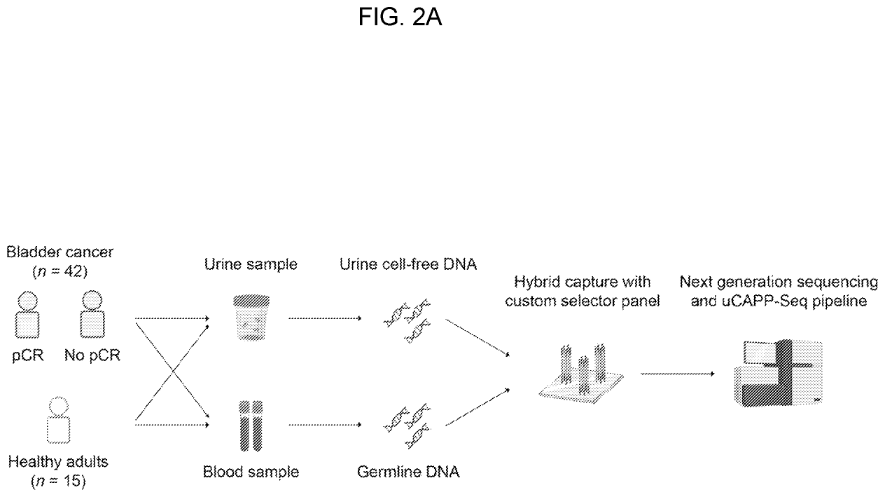

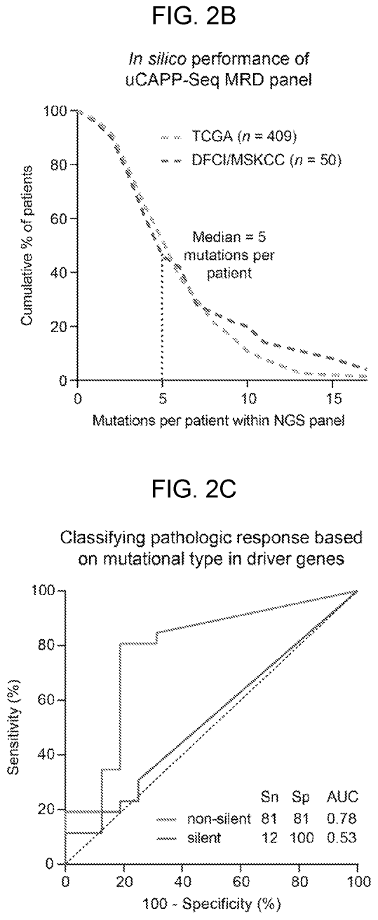

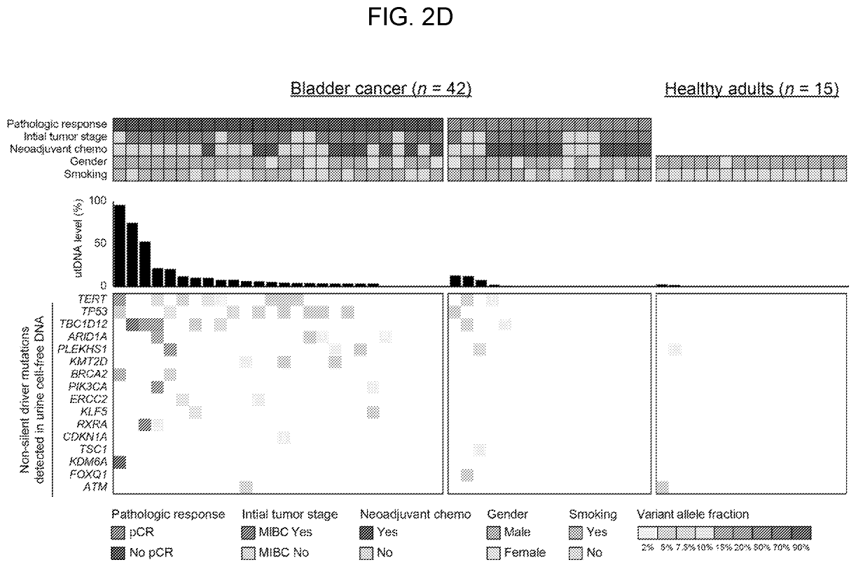

Urine cfDNA was profiled and peripheral blood germline samples collected from 42 patients were matched with localized bladder cancer and 15 healthy donors (see e.g., FIG. 2A). The bladder cancer cohort was comprised of 76% ( 32/42) males and 64% ( 27/42) smokers, and the average age was 69 years. A total of 76% ( 32/42) of patients had a confirmed diagnosis of MIBC, 59% of whom received neoadjuvant chemotherapy, while the remaining 24% had high-risk NMIBC (see e.g., TABLE 1 and TABLE 2). No patients had any other known active cancer diagnoses at the time of surgery.

TABLE 1
Characteristics of enrolled bladder cancer patients
and healthy adults.

| Characteristics | Number | Percentage |
|---|---|---|
| Bladder cancer | | |
| patients (n = 42) | | |
| Sex, n (%) | | |
| Male | 32 | 76 |
| Female | 10 | 24 |
| Age (years) | 69 (50-86) | — |
| Ethnicity, n (%) | | |
| White | 40 | 95 |
| Non-White | 2 | 5 |
| Follow-up time (days) | 183 (27-200) | — |
| Smoking, n (%) | | |
| Yes | 27 | 64 |
| No | 15 | 35 |
| Pack-years | 20 (0-56) | — |
| Pretreatment T stage^{a}, | | |
| n (%) | | |
| Ta | 2 | 5 |
| Tis | 1 | 2 |
| T1 | 7 | 17 |
| T2 | 26 | 62 |
| T3 | 6 | 14 |
| T4 | 0 | 0 |
| Neoadjuvant | | |
| chemotherapy | | |
| received, n (%) | | |
| Yes | 19 | 45 |
| ddMVAC | 4 | 21 |
| Gemcitabine/cisplatin | 10 | 53 |
| Gemcitabine/ | 1 | 5 |
| carboplatin | | |
| Carboplatin/paclitaxel | 2 | 11 |
| Treatment switch^{b} | 1 | 5 |
| Unknown regimen type | 2 | 11 |
| No | 23 | 55 |
| PCR^{c}, n (%) | 16 | 37 |
| Histology, n (%) | | |
| Urothelial | 36 | 85 |
| Squamous | 3 | 7 |
| Other | 3 | 7 |
| Healthy adults^{d} (n = 27) | | |
| Sex, n (%) | | |
| Male | 22 | 81 |
| Female | 5 | 19 |
| Age (years) | 31 (20-65) | — |

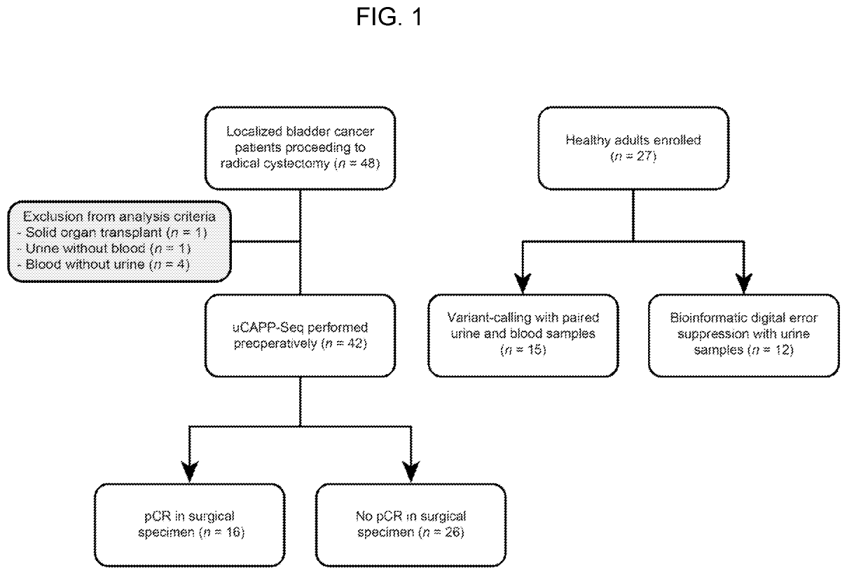

ªT staging was performed at the time of pretreatment TURBT using the AJCC 8 criteria.
^{b}Treatment switch: 1 cycle of carboplatin/paclitaxel and 3 cycles of gemcitabine/carboplatin (switched due to cutaneous reaction to paclitaxel).
^{c}pCR was defined as T0N0, TaN0, or TisN0 (see Methods: Pathologic response assessment).
^{d}Urine and peripheral blood samples from 15 healthy adults were collected to assess specificity of the variant calling approach (see e.g., FIG. 1, FIG. 2A, and Methods). Urine samples were collected from another 12 healthy adults to use for iDES (see e.g., FIG. 1 and Methods).
AJCC 8, American Joint Committee on Cancer 8th edition; ddMVAC, dose-dense methotrexate, vinblastine, doxorubicin, and cisplatin; iDES, integrated digital error suppression; pCR, pathologic complete response; TURBT, transurethral resection of bladder tumor.

TABLE 2
Patient-level characteristics in the bladder cancer cohort.

| | | | | | Initial | | Neoadjuvant | |
|---|---|---|---|---|---|---|---|---|
| Patient | | | | | “T” | De | chemo- | AJCC 8 |
| ID | Age | Sex | Ethnicity | Histology | stage^{a} | novo^{b} | therapy | stage^{c} |
| BC- | 63 | Male | White | urothelial | T2 | Yes | Yes | ypT0N2 |
| 1045 | | | | carcinoma | | | | |
| BC- | 78 | Male | White | urothelial | T2 | Yes | No | pT1N0 |
| 1058 | | | | carcinoma | | | | |
| BC- | 67 | Male | White | urothelial | T2 | Yes | Yes | ypT0N0 |
| 1063 | | | | carcinoma | | | | |
| BC- | 65 | Male | White | urothelial | T2 | Yes | Yes | ypT3aN0 |
| 1077 | | | | carcinoma | | | | |
| BC- | 68 | Male | White | urothelial | T2 | Yes | Yes | ypT2N0 |
| 1105 | | | | carcinoma | | | | |
| BC- | 59 | Female | White | urothelial | T2 | No | Yes | ypT3aN1 |
| 1135 | | | | carcinoma | | | | |
| BC- | 79 | Male | Non- | urothelial | T1 | Yes | No | pT3aN0 |
| 1171 | | | White | carcinoma | | | | |
| BC- | 72 | Male | White | urothelial | T2 | Yes | No | pT3aN2 |
| 1182 | | | | carcinoma | | | | |
| BC- | 65 | Male | White | urothelial | T2 | Yes | Yes | ypTisN0 |
| 1185 | | | | carcinoma | | | | |
| BC- | 82 | Female | White | urothelial | T1 | Yes | No | pT3aN0 |
| 1186 | | | | carcinoma | | | | |
| BC- | 64 | Male | White | urothelial | T3 | No | Yes | ypTaN0 |
| 1065 | | | | carcinoma | | | | |
| BC- | 61 | Male | White | urothelial | T3 | Yes | No | pT3aN2 |
| 1069 | | | | carcinoma, | | | | |
| | | | | squamous | | | | |
| | | | | differentiation | | | | |
| BC- | 63 | Female | White | urothelial | T2 | Yes | Yes | ypT0N0 |
| 1050 | | | | carcinoma | | | | |
| BC- | 60 | Male | White | urothelial | T2 | Yes | Yes | ypTisN0 |
| 1064 | | | | carcinoma | | | | |
| BC- | 68 | Male | White | urothelial | T2 | Yes | No | pT4aN2 |
| 1071 | | | | carcinoma | | | | |
| BC- | 75 | Male | White | urothelial | at | Yes | No | pT3aN0 |
| 1076 | | | | carcinoma, | least | | | |
| | | | | trophoblastic | T2 | | | |
| | | | | differentiation | | | | |
| BC- | 66 | Male | White | urothelial | T3 | Yes | Yes | ypT3aN0 |
| 1083 | | | | carcinoma | | | | |
| BC- | 72 | Male | White | urothelial | at | No | No | pT4aN1 |
| 1085 | | | | carcinoma | least | | | |
| | | | | | T2 | | | |
| BC- | 83 | Male | White | urothelial | T1 | No | No | pT3aN0 |
| 1090 | | | | carcinoma | | | | |
| BC- | 86 | Male | White | urothelial | at | Yes | No | pT3aN2 |
| 1096 | | | | carcinoma | least | | | |
| | | | | | T2 | | | |
| BC- | 53 | Female | White | urothelial | T1 | No | No | pTisN0 |
| 1108 | | | | carcinoma | | | | |
| BC- | 83 | Male | White | urothelial | Tis | No | No | pTisN0 |
| 1109 | | | | carcinoma | | | | |
| BC- | 72 | Female | Non- | papillary | Ta | No | No | pT2bN1 |
| 1116 | | | White | urothelial | | | | |
| | | | | carcinoma | | | | |
| BC- | 51 | Male | White | urothelial | T3 | No | Yes | ypT3bN2 |
| 1132 | | | | carcinoma | | | | |
| BC- | 77 | Male | White | papillary | T2 | Yes | Yes | ypT2aN0 |
| 1062 | | | | urothelial | | | | |
| | | | | carcinoma | | | | |
| BC- | 70 | Female | White | papillary | T1 | No | No | pTaN0 |
| 1140 | | | | urothelial | | | | |
| | | | | carcinoma | | | | |
| BC- | 71 | Male | White | urothelial | at | Yes | No | pT3aN1 |
| 1147 | | | | carcinoma | least | | | |
| | | | | | T2 | | | |
| BC- | 55 | Male | White | squamous | at | Yes | Yes | ypT2bN0 |
| 1104 | | | | cell | least | | | |
| | | | | carcinoma | T2 | | | |
| BC- | 83 | Male | White | papillary | Ta | No | No | pT1N0 |
| 1088 | | | | urothelial | | | | |
| | | | | carcinoma | | | | |
| BC- | 72 | Male | White | urothelial | T2 | No | No | pT2aN0 |
| 1188 | | | | carcinoma | | | | |
| BC- | 68 | Male | White | urothelial | T1 | No | No | pT0N0 |
| 1111 | | | | carcinoma | | | | |
| BC- | 68 | Female | White | papillary | focal | No | No | pTaN0 |
| 1133 | | | | urothelial | T2 | | | |
| | | | | carcinoma | | | | |
| BC- | 69 | Male | White | urothelial | T1 | No | No | pTisN0 |
| 1174 | | | | carcinoma | | | | |
| BC- | 60 | Male | White | urothelial | at | Yes | Yes | ypT0N0 |
| 1196 | | | | carcinoma | least | | | |
| | | | | | T2 | | | |
| BC- | 64 | Male | White | urothelial | at | Yes | Yes | ypT0N0 |
| 1197 | | | | carcinoma | least | | | |
| | | | | | T2 | | | |
| BC- | 63 | Female | White | urothelial | at | Yes | Yes | ypT0N1 |
| 1198 | | | | carcinoma, | least | | | |
| | | | | squamous | T2 | | | |
| | | | | differentiation | | | | |
| BC- | 66 | Female | White | urothelial | at | No | No | pT3aN0 |
| 1202 | | | | carcinoma | least | | | |
| | | | | | T2 | | | |
| BC- | 82 | Male | White | papillary | T2 | Yes | Yes | ypTaN0 |
| 1203 | | | | urothelial | | | | |
| | | | | carcinoma | | | | |
| BC- | 64 | Male | White | urothelial | T3 | Yes | Yes | ypTisN0 |
| 1205 | | | | carcinoma | | | | |
| BC- | 65 | Female | White | squamous | T3 | Yes | No | pT4bN1 |
| 1206 | | | | cell | | | | |
| | | | | carcinoma | | | | |
| BC- | 76 | Male | White | urothelial | T2 | Yes | Yes | ypT0N0 |
| 1207 | | | | carcinoma | | | | |
| BC- | 50 | Male | White | squamous | T2 | Yes | No | pT3aN0 |
| 1209 | | | | cell | | | | |
| | | | | carcinoma | | | | |

| | Pathologic | | | | |
|---|---|---|---|---|---|
| Patient ID | CR^{d} | Smoker | Pack-years | Progression^{e} | Death^{e} |
| BC-1045 | No | Yes | 45 | No | No |
| BC-1058 | No | No | 0 | No | No |
| BC-1063 | Yes | No | 0 | No | No |
| BC-1077 | No | Yes | 30 | No | No |
| BC-1105 | No | Yes | 20 | No | No |
| BC-1135 | No | Yes | 30 | No | No |
| BC-1171 | No | Yes | 45 | No | No |
| BC-1182 | No | Yes | 30 | No | No |
| BC-1185 | Yes | Yes | 0.75 | No | No |
| BC-1186 | No | No | 0 | No | No |
| BC-1065 | Yes | No | 0 | No | No |
| BC-1069 | No | Yes | 30 | No | No |
| BC-1050 | Yes | Yes | 40 | No | No |
| BC-1064 | Yes | Yes | 2 | No | No |
| BC-1071 | No | No | 0 | Yes | Yes |
| BC-1076 | No | Yes | 25 | No | No |
| BC-1083 | No | Yes | 22 | Yes | Yes |
| BC-1085 | No | Yes | 56 | Yes | Yes |
| BC-1090 | No | No | 0 | No | No |
| BC-1096 | No | No | 0 | Yes | No |
| BC-1108 | Yes | No | 0 | No | No |
| BC-1109 | Yes | Yes | 15 | No | No |
| BC-1116 | No | Yes | 30 | No | No |
| BC-1132 | No | No | 0 | No | No |
| BC-1062 | No | Yes | 20 | No | No |
| BC-1140 | Yes | Yes | 5.5 | No | No |
| BC-1147 | No | No | 0 | No | No |
| BC-1104 | No | No | 0 | No | No |
| BC-1088 | No | No | 0 | No | No |
| BC-1188 | No | Yes | 20 | No | No |
| BC-1111 | Yes | Yes | 30 | No | No |
| BC-1133 | Yes | Yes | 10.2 | No | No |
| BC-1174 | Yes | Yes | 40 | No | No |
| BC-1196 | Yes | Yes | 45 | No | No |
| BC-1197 | Yes | No | 42 | No | No |
| BC-1198 | No | Yes | 38 | No | No |
| BC-1202 | No | Yes | 30 | No | No |
| BC-1203 | Yes | Yes | 42 | No | No |
| BC-1205 | Yes | Yes | 50 | No | No |
| BC-1206 | No | No | 0 | Yes | Yes |
| BC-1207 | Yes | Yes | 30 | No | No |
| BC-1209 | No | No | 0 | Yes | No |

^{a}Staging was performed at the time of pre-treatment TURBT using AJCC 8 criteria.
^{b}De novo refers to whether this instance of localized bladder cancer was a new diagnosis.
^{c}Staging was performed at the time of radical cystectomy using AJCC 8 criteria, with “y” indicating the patient received neoadjuvant chemotherapy.
^{d}Pathologic CR status was defined as T0, Ta or Tis at the time of radical cystectomy (see e.g., Methods: Pathologic Response Assessment).
^{e}Early progression and survival were assessed within 200 days of radical cystectomy (see Methods: Power and Statistical Analyses).
AJCC 8 = American Joint Committee on Cancer 8th edition
CR = complete response
TURBT = transurethral resection of bladder tumor

Optimization of Urine Cell-Free DNA Processing

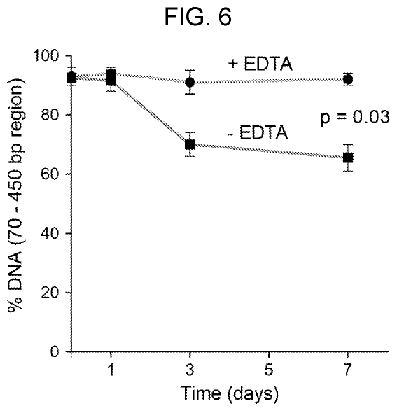

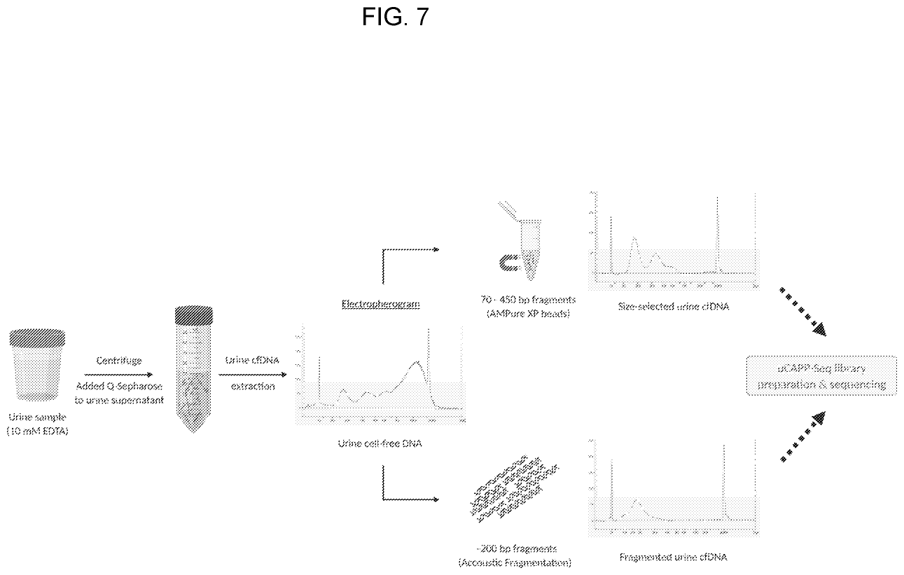

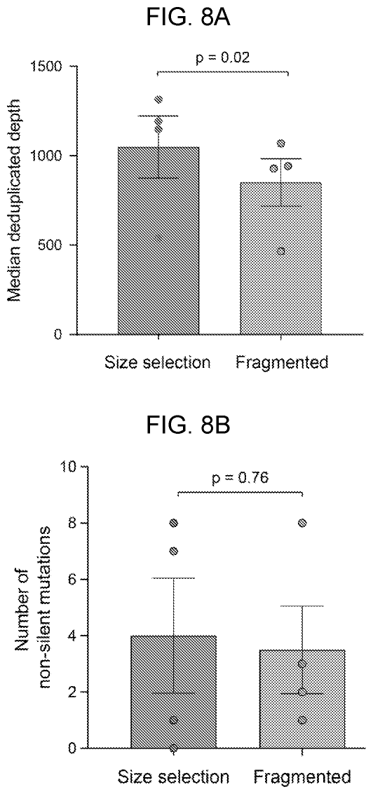

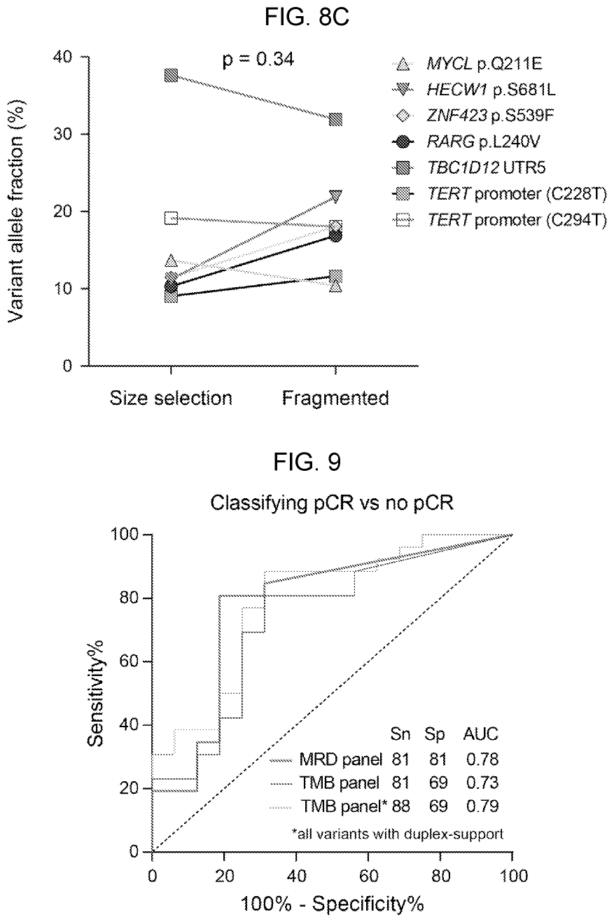

All patient urine samples were collected preoperatively just prior to the time of radical cystectomy with the addition of EDTA to preserve the stability of urine cfDNA at room temperature (see e.g., FIG. 6). Two different methodologies of urine cfDNA processing were also compared prior to urine cfDNA library preparation: standard protocol of acoustic fragmentation versus in vitro size selection of smaller fragments (see e.g., FIG. 7). Although significantly higher median deduplicated depth was achieved (see e.g., FIG. 8A) with the size selection method compared to the fragmentation protocol in 4 MIBC patients, there were no significant differences in the number of non-silent mutations detected (see e.g., FIG. 8B) or in the variant allele fraction levels among common non-silent mutations (see e.g., FIG. 8C) detected by the 2 techniques.

Development and Validation of MRD Gene Panel for utDNA Detection

A noninvasive approach was used without prior sequencing of tumor tissue using uCAPP-Seq to profile mutations detected in urine. For a targeted evaluation of defined consensus regions, a focused MRD gene panel encompassing 49 consensus driver genes frequently mutated in bladder cancer was used (see e.g., TABLE 3).

TABLE 3
Genes fully or partially covered in the MRD panel.

| | | | | | |
|---|---|---|---|---|---|
| ARID1A | CDKN1A | ERCC5 | KMT2C | POLE | TP53 |
| ASXL2 | CDKN2A | FANCC | KMT2D | RB1 | TSC1 |
| ATM | CREBBP | FBXW7 | KRAS | RHOA | TXNIP |
| ATR | ELF3 | FGFR3 | MDM2 | RHOB | ZFP36L1 |
| BRCA1 | EP300 | FOXA1 | NF1 | RXRA | |
| BRCA2 | ERBB2 | FOXQ1 | NFE2L2 | SPTAN1 | |
| BTG2 | ERBB3 | HRAS | PAIP1 | STAG2 | |
| CCND1 | ERBB4 | KDM6A | PIK3CA | TBC1D12 | |
| CCND3 | ERCC2 | KLF5 | PLEKHS1 | TERT | |

MRD = minimal residual disease

These driver genes were selected from previous literature with comprehensive genomic characterization of bladder cancer. In silico application of this MRD gene panel to 409 MIBC tumors profiled by TCGA and 50 MIBC tumors profiled by DFCI/MSKCC predicted that 96% of patients had mutations detectable within the gene panel space with a median of 5 mutations (non-silent and silent) detected per patient (see e.g., FIG. 2B).

The MRD panel was then tested for concordance of mutations detected in urine compared to tumor tissue. Five patients had paired urine and tumor tissue available for uCAPP-Seq using the MRD panel. Three patients had radical cystectomy tissue available, while 2 had transurethral resection of bladder tumor tissue from the time of diagnosis. Of note, none of these tissue samples had been affected by neoadjuvant chemotherapy. The concordance between urine- and tumor-derived mutations, determined in these 5 localized bladder cancer patients by uCAPP-Seq, was 85% (see e.g., TABLE 4).

TABLE 4
Mutational concordance between tumor tissue and urine cell-
free DNA.

| Patient | | Chromo- | | Mutant | Reference |
|---|---|---|---|---|---|
| ID | Gene | some | Position^{a} | allele | allele |
| BC-1071 | KLF5 | chr13 | 73649902 | C | G |
| BC-1071 | TBC1D12 | chr10 | 96162370 | A | G |
| BC-1085 | RXRA | chr9 | 137328351 | T | C |
| BC-1085 | TBC1D12 | chr10 | 96162368 | T | C |
| BC-1085 | KMT2D | chr12 | 49427116 | C | G |
| BC-1088 | TERT | chr5 | 1295228 | A | G |
| BC-1088 | TBC1D12 | chr10 | 96162368 | T | C |
| BC-1186 | ARID1A | chr1 | 27057874 | T | C |
| BC-1186 | TP53 | chr17 | 7579431 | T | C |
| BC-1206 | TERT | chr5 | 1295228 | A | G |
| BC-1206 | TP53 | chr17 | 7578275 | A | G |
| BC-1206 | ARID1A | chr1 | 27099915 | T | G |
| BC-1206 | TP53 | chr17 | 7578275 | A | G |

| Patient | Mutation | Detected | Detected |
|---|---|---|---|
| ID | type^{b} | in Urine^{c} | in Tumor^{d} |
| BC-1071 | nonsynonymous | Yes | No |
| | SNV | | |
| BC-1071 | UTR5 | Yes | No |
| BC-1085 | nonsynonymous | Yes | Yes |
| | SNV | | |
| BC-1085 | UTR5 | Yes | Yes |
| BC-1085 | nonsynonymous | Yes | Yes |
| | SNV | | |
| BC-1088 | upstream | Yes | Yes |
| BC-1088 | UTR5 | Yes | Yes |
| BC-1186 | stopgain | Yes | Yes |
| BC-1186 | nonsynonymous | Yes | Yes |
| | SNV | | |
| BC-1206 | upstream | Yes | Yes |
| BC-1206 | stopgain | Yes | Yes |
| BC-1206 | nonsynonymous | Yes | Yes |
| | SNV | | |
| BC-1206 | stopgain | Yes | Yes |

^{a}Genomic coordinates are per the GRCH37/hg 19 (February 2009) genome assembly (see Methods: DNA Library Construction and Sequencing).
^{b}Mutational type was determined using ANNOVAR following CAPP-Seq analysis (see Methods: Duplex-Supported Variant-Calling for utDNA MRD Analysis).
^{c}Detection status of identified mutations within urine cell-free DNA using the CAPP-Seq monitoring pipeline (see Methods: Duplex-Supported Variant-Calling for utDNA MRD Analysis).
^{d}Detection status of identified mutations within tumor tissue DNA using the CAPP-Seq monitoring pipeline (see Methods: Duplex-Supported Variant-Calling for utDNA MRD Analysis).
CAPP-Seq = Cancer Personalized Profiling by Deep Sequencing
chr = chromosome
SNV = single nucleotide variant
UTR = untranslated region

Non-Silent Mutations Classify Pathologic Response

In the cohort, 38% ( 16/42) of patients achieved a pCR (n=16), while 62% ( 26/42) had residual disease detected in their surgical sample (denoted as no pCR; n=26). There were no significant differences between pCR and no pCR patients with regard to age, sex, ethnicity, smoking status, pretreatment tumor stage, receipt of neoadjuvant chemotherapy, or tumor histology (see e.g., TABLE 5).

TABLE 5
Characteristics of patients according to pathologic
response at surgery.

| | Pathologic | No pathologic | |
|---|---|---|---|
| | complete | complete | |
| Characteristic | response | response | |
| | (n = 16) | (n = 26) | p value* |
| Sex | | | |
| Male | 12 (75%) | 20 (77%) | >0.99 |
| Female | 4 (25%) | 6 (23%) | |
| Age (years) | 67 (53-83) | 69 (50 - 86) | 0.47 |
| Ethnicity, n | | | |
| (%) | | | |
| White | 16 (100%) | 24 (92%) | 0.51 |
| Non-White | 0 (0%) | 2 (8%) | |
| Follow-up | 186 (71-200) | 183 (27-200) | 0.64 |
| time (days) | | | |
| Smoking, n | | | |
| (%) | | | |
| Yes | 12 (75%) | 15 (58%) | 0.33 |
| No | 4 (25%) | 11 (42%) | |
| Pack-years | 23 (0-50) | 20 (0-56) | 0.25 |
| Pre-treatment | | | |
| T stage^{a}, n (%) | | | |
| Ta | 0 (0%) | 2 (8%) | 0.51 |
| Tis | 1 (6%) | 0 (0%) | 0.40 |
| T1 | 4 (25%) | 3 (12%) | 0.41 |
| T2 | 9 (56%) | 17 (65%) | 0.50 |
| T3 | 2 (13%) | 4 (15%) | >0.99 |
| Neoadjuvant | | | |
| Chemotherapy | | | |
| Received, n (%) | | | |
| Yes | 10 (62%) | 9 (35%) | 0.11 |
| No | 6 (38%) | 17 (65%) | |
| Urinary DNA^{b}, | | | |
| median (range) | | | |
| urine cell-free | 23.9 (2.2-136.5) | 44.1 (1.0-391.2) | 0.17 |
| DNA (ng/ml) | | | |
| utDNA level | 0 (0-12.5) | 4.3 (0-95.2) | 0.002 |
| [max VAF (%)] | | | |
| Histology, n | | | |
| (%) | | | |
| Urothelial | 16 (100%) | 20 (83.3%) | 0.07 |
| Squamous | 0 (0%) | 3 (11.5%) | 0.28 |
| Other | 0 (0%) | 3 (11.5%) | 0.28 |

^{a}T staging was performed at the time of pre-treatment TURBT using AJCC 8 criteria.
^{b}Urinary DNA levels were quantified by total cell-free DNA concentration and the utDNA level (maximum variant allele fraction) (see Methods: Urine Cell-free DNA Isolation and Quantification, and Methods: Duplex-Supported Variant-Calling).
*p values were calculated using Fisher's exact test for categorical variables, unpaired Student's t-test for normally distributed continuous variables, and Mann-Whitney U test for non-normally distributed continuous variables. Normality was assessed using the Shapiro Wilk test with a significance level of 0.05.
AJCC 8 = American Joint Committee on Cancer 8th edition
max = maximum
utDNA = urine tumor DNA
VAF = variant allele fraction

Furthermore, there was no significant difference in the concentration of urine cfDNA obtained between the 2 groups of patients.

The MRD gene panel was applied to the preoperative urine of these patients and a total of 52 non-silent mutations and 17 silent mutations was detected (see e.g., TABLE 6).

TABLE 6
Mutations detected at the preoperative MRD time point in urine using MRD panel.

| Patient | | | | | Mutant | Reference |
|---|---|---|---|---|---|---|
| ID | Gene | Chromosome | Position | Mutation type^{b} | allele | allele |
| BC-1045 | KMT2D | chr12 | 49433287 | stopgain | T | C |
| BC-1045 | PLEKHS1 | chr10 | 115511590 | intronic | C | G |
| BC-1058 | ATM | chr11 | 108198371 | splicing | A | G |
| BC-1058 | KMT2D | chr12 | 49432335 | nonsynonymous | A | G |
| BC-1062 | PLEKHS1 | chr10 | 115511593 | intronic | T | C |
| BC-1069 | TERT | chr5 | 1295228 | upstream | A | G |
| BC-1069 | ERCC2 | chr19 | 45867687 | nonsynonymous | C | T |
| BC-1071 | KLF5 | chr13 | 73649902 | nonsynonymous | C | G |
| BC-1071 | TBC1D12 | chr10 | 96162370 | UTR5 | A | G |
| BC-1076 | KMT2D | chr12 | 49435925 | stopgain | C | G |
| BC-1076 | CDKN1A | chr6 | 36652140 | nonsynonymous | A | G |
| BC-1076 | TP53 | chr17 | 7579406 | stopgain | C | G |
| BC-1076 | TERT | chr5 | 1295228 | upstream | A | G |
| BC-1077 | TERT | chr5 | 1295228 | upstream | A | G |
| BC-1083 | TP53 | chr17 | 7578479 | nonsynonymous | A | G |
| BC-1083 | ERCC2 | chr19 | 45860733 | nonsynonymous | A | C |
| BC-1085 | TBC1D12 | chr10 | 96162368 | UTR5 | T | C |
| BC-1085 | RXRA | chr9 | 137328351 | nonsynonymous | T | C |
| BC-1088 | TERT | chr5 | 1295228 | upstream | A | G |
| BC-1088 | TBC1D12 | chr10 | 96162368 | UTR5 | T | C |
| BC-1090 | TERT | chr5 | 1295228 | upstream | A | G |
| BC-1096 | TBC1D12 | chr10 | 96162368 | UTR5 | T | C |
| BC-1104 | TP53 | chr17 | 7579310 | splicing | C | A |
| BC-1105 | ARID1A | chr1 | 27057795 | nonsynonymous | T | G |
| BC-1111 | TSC1 | chr9 | 135781419 | stopgain | A | G |
| BC-1111 | PLEKHS1 | chr10 | 115511593 | intronic | T | C |
| BC-1133 | TP53 | chr17 | 7577545 | nonsynonymous | C | T |
| BC-1135 | TERT | chr5 | 1295228 | upstream | A | G |
| BC-1171 | BRCA2 | chr13 | 32972638 | nonsynonymous | A | G |
| BC-1171 | TP53 | chr17 | 7578444 | nonsynonymous | C | G |
| BC-1171 | TERT | chr5 | 1295228 | upstream | A | G |
| BC-1171 | KDM6A | chrX | 44969323 | splicing | A | G |
| BC-1174 | FOXQ1 | chr6 | 1313436 | nonsynonymous | T | C |
| BC-1174 | TBC1D12 | chr10 | 96162368 | UTR5 | T | C |
| BC-1174 | TERT | chr5 | 1295228 | upstream | A | G |
| BC-1182 | PLEKHS1 | chr10 | 115511593 | intronic | T | C |
| BC-1182 | TP53 | chr17 | 7578431 | stopgain | A | G |
| BC-1182 | BRCA2 | chr13 | 32910940 | nonsynonymous | T | A |
| BC-1185 | TERT | chr5 | 1295228 | upstream | A | G |
| BC-1186 | ARID1A | chr1 | 27057874 | stopgain | T | C |
| BC-1186 | TP53 | chr17 | 7579431 | nonsynonymous | T | C |
| BC-1188 | RXRA | chr9 | 137328351 | nonsynonymous | T | C |
| BC-1188 | PIK3CA | chr3 | 178936091 | nonsynonymous | A | G |
| BC-1188 | ARID1A | chr1 | 27092809 | stopgain | T | C |
| BC-1188 | TBC1D12 | chr10 | 96162370 | UTR5 | A | G |
| BC-1188 | TERT | chr5 | 1295228 | upstream | A | G |
| BC-1202 | KLF5 | chr13 | 73636861 | nonsynonymous | A | G |
| BC-1202 | PIK3CA | chr3 | 178936029 | nonsynonymous | A | G |
| BC-1205 | TBC1D12 | chr10 | 96162370 | UTR5 | A | G |
| BC-1206 | TP53 | chr17 | 7578275 | stopgain | A | G |
| BC-1206 | ARID1A | chr1 | 27099915 | nonsynonymous | T | G |
| BC-1209 | TP53 | chr17 | 7578406 | nonsynonymous | T | C |

| | | | | OncoKB annotation | | Unique | |
|---|---|---|---|---|---|---|---|
| | Amino | Amino | | | Investigational | sequencing | VAF |
| Patient | acid | acid | COSMIC | Significance | drug | depth^{f} | (%)^{g} |
| ID | change | position | annotation^{c} | level^{d} | available^{e} | 1078 | 2.41 |
| BC-1045 | TRP > stop | 2720 | . | N/A | N/A | 1247 | 2.00 |
| BC-1045 | none | . | . | N/A | N/A | 682 | 5.87 |
| BC-1058 | none | . | . | N/A | N/A | 1258 | 0.64 |
| BC-1058 | SER > LEU | 2935 | . | N/A | N/A | 1268 | 2.37 |
| BC-1062 | none | . | . | N/A | N/A | 1275 | 11.61 |
| BC-1069 | none | . | . | N/A | N/A | 1522 | 9.40 |
| BC-1069 | ASN > SER | 214/238 | COSM1750977, | 3A | YES | 792 | 10.10 |
| | | | COSM418170 | | | | |
| BC-1071 | ASP > HIS | 327/418 | . | N/A | N/A | 2040 | 2.75 |
| BC-1071 | none | . | . | N/A | N/A | 845 | 3.20 |
| BC-1076 | SER > stop | 2019 | . | N/A | N/A | 1535 | 2.02 |
| BC-1076 | GLU > LYS | 88/122 | . | N/A | N/A | 1962 | 3.82 |
| BC-1076 | SER > stop | 55/94 | COSM45653 | N/A | N/A | 4246 | 2.78 |
| BC-1076 | none | . | . | N/A | N/A | 1275 | 9.65 |
| BC-1077 | none | . | . | N/A | N/A | 2073 | 5.64 |
| BC-1083 | PRO > SER | 19/112/151 | COSM984961, | N/A | N/A | 3958 | 0.53 |
| | | | COSM1640859, | | | | |
| | | | COSM3378358, | | | | |
| | | | COSM984963, | | | | |
| | | | COSM10905, | | | | |
| | | | COSM984959, | | | | |
| | | | COSM984962 | | | | |
| BC-1083 | GLY > VAL | 459 | . | N/A | N/A | 577 | 18.20 |
| BC-1085 | none | . | . | N/A | N/A | 734 | 52.18 |
| BC-1085 | SER > PHE | 330/400/427 | COSM1314610 | N/A | N/A | 1395 | 1.08 |
| BC-1088 | none | . | . | N/A | N/A | 799 | 7.38 |
| BC-1088 | none | . | . | N/A | N/A | 1186 | 3.46 |
| BC-1090 | none | . | . | N/A | N/A | 393 | 74.30 |
| BC-1096 | none | . | . | N/A | N/A | 417 | 2.40 |
| BC-1104 | none | . | COSM46043 | N/A | N/A | 1929 | 0.41 |
| BC-1105 | GLN > HIS | 501 | . | N/A | N/A | 922 | 1.84 |
| BC-1111 | GLN > stop | 465/516/515 | COSM307636 | 3B | YES | 684 | 7.31 |
| BC-1111 | none | . | . | N/A | N/A | 657 | 12.48 |
| BC-1133 | MET > VAL | 114/87/246 | COSM251429, | N/A | N/A | 788 | 4.82 |
| | | | COSM3958808, | | | | |
| | | | COSM251430, | | | | |
| | | | COSM251431, | | | | |
| | | | COSM1726382, | | | | |
| | | | COSM43555 | | | | |
| BC-1135 | none | . | . | N/A | N/A | 1738 | 15.65 |
| BC-1171 | GLU > LYS | 3330 | . | N/A | N/A | 1859 | 0.27 |
| BC-1171 | ILE > MET | 30/123/162 | COSM44125 | N/A | N/A | 2988 | 24.80 |
| BC-1171 | none | . | . | N/A | N/A | 438 | 95.21 |
| BC-1171 | none | . | COSM1319435 | N/A | N/A | 735 | 3.67 |
| BC-1174 | SER > PHE | 166 | . | N/A | N/A | 898 | 11.80 |
| BC-1174 | none | . | . | N/A | N/A | 1754 | 5.59 |
| BC-1174 | none | . | . | N/A | N/A | 523 | 19.89 |
| BC-1182 | none | . | . | N/A | N/A | 902 | 10.20 |
| BC-1182 | GLN > stop | 8/35/128/167 | COSM121084, | N/A | N/A | 489 | 6.34 |
| | | | COSM3937613, | | | | |
| | | | COSM121081, | | | | |
| | | | COSM121083, | | | | |
| | | | COSM121082, | | | | |
| | | | COSM11333, | | | | |
| | | | COSM2744872 | | | | |
| BC-1182 | GLU > ASP | 816 | . | N/A | N/A | 1503 | 1.66 |
| BC-1185 | none | . | . | N/A | N/A | 2003 | 2.65 |
| BC-1186 | GLN > stop | 528 | COSM426004 | N/A | N/A | 1053 | 3.42 |
| BC-1186 | ALA > THR | 47/86 | . | N/A | N/A | 654 | 1.22 |
| BC-1188 | SER > PHE | 330/400/427 | COSM1314610 | N/A | N/A | 420 | 20.95 |
| BC-1188 | GLU > LYS | 545 | COSM763, | 3B | YES | 790 | 17.34 |
| | | | COSM125370 | | | | |
| BC-1188 | GLN > stop | 944 | COSM130413 | N/A | N/A | 656 | 19.05 |
| BC-1188 | none | . | . | N/A | N/A | 1074 | 8.19 |
| BC-1188 | none | . | . | N/A | N/A | 550 | 2.36 |
| BC-1202 | CYS > TYR | 284/375 | . | N/A | N/A | 525 | 1.52 |
| BC-1202 | ARG > LYS | 524 | COSM53245 | N/A | N/A | 1214 | 0.74 |
| BC-1205 | none | . | . | N/A | N/A | 2112 | 2.84 |
| BC-1206 | GLN > stop | 33/60/153/192 | COSM10733, | N/A | N/A | 2080 | 0.91 |
| | | | COSM3370929, | | | | |
| | | | COSM117948, | | | | |
| | | | COSM117949, | | | | |
| | | | COSM117947, | | | | |
| | | | COSM117946, | | | | |
| | | | COSM1649390 | | | | |
| BC-1206 | GLY > VAL | 1265 | . | N/A | N/A | 810 | 7.28 |
| BC-1209 | ARG > HIS | 43/16/136/175 | COSM3355994, | N/A | N/A | 1078 | 2.41 |
| | | | COSM1640851, | | | | |
| | | | COSM99024, | | | | |
| | | | COSM99023, | | | | |
| | | | COSM10648, | | | | |
| | | | COSM99914, | | | | |
| | | | COSM99022 | | | | |

^{a}Genomic coordinates are per the GRCH37/hg19 genome assembly (see Methods: DNA Library Construction and Sequencing).
^{b}Mutational type was determined using ANNOVAR following uCAPP-Seq analysis (see Methods: Duplex-Supported Variant-Calling for utDNA MRD Analysis).
^{c}COSMIC identifiers are included for variants that are indexed in the COSMIC70 database.
^{d}Level of evidence (v2) is included for variants indexed in the OncoKB database (see Methods: Identifying Actionable Mutations from utDNA Analysis).
^{e}Investigational drug potentially available for the specific alteration, based on OncoKB level of evidence (v2) of 3B or higher (see Methods: Identifying Actionable Mutations from utDNA Analysis).
^{f}Deduplicated sequencing depth at the specified genomic position in urine cell-free DNA after uCAPP-Seq (see Methods: DNA Library Construction and Sequencing).
^{g}Variant allele fraction calculated as the percentage of mutant reads at the specified genomic position in urine cell-free DNA following uCAPP-Seq analysis (see Methods: DNA Library Construction and Sequencing).
chr = chromosome
MRD = minimal residual disease
N/A = not available
SNV = single nucleotide variant
uCAPP-Seq = urine Cancer Personalized Profiling by Deep Sequencing
UTR = untranslated region
VAF = variant allele fraction

To have high confidence in the noninvasive variant calling, only mutations detected in urine cfDNA with duplex support and with no read support in matched peripheral blood germline samples were considered. Using the mutation with the highest variant allele fraction to represent the utDNA level, the strength of association between pathologic response and utDNA detected from either non-silent or silent mutations was compared. The ROC curve for non-silent mutations achieved a sensitivity of 81% and specificity of 81% (AUC=0.78; 95% CI: 0.62 to 0.93), whereas the ROC curve for silent mutations achieved a sensitivity of only 12% (AUC=0.53; 95% CI: 0.35 to 0.71) (see e.g., FIG. 2C). These results indicate that non-silent mutations classify pathologic response much more accurately than silent mutations, potentially because silent mutations are more likely to represent background field effect.

Consistent with increased background noise due to field effect, variant calling of non-silent, duplex-supported mutations within an expanded panel of 536 genes covering 387 kb of genomic space did not offer increased sensitivity and had inferior specificity (see e.g., FIG. 9). The sensitivity only increased when variant calling was expanded to all duplex-supported mutations (silent or non-silent) within the expanded gene panel, but this also came at the expense of lower specificity. As a result, variant calling of non-silent, duplex-supported mutations within the driver gene-focused MRD panel yielded the best balance of sensitivity and specificity for classifying pathologic response in the cohort.

No pCR is Associated with Higher utDNA Levels than pCR

Focusing only on non-silent mutations in the MRD gene panel, it was observed that patients with no pCR carried significantly more mutations (median of 2 mutations per patient) than patients with pCR (median of 0 mutations per patient). Urine cfDNA harbored a lower median number of mutations at the MRD time point than the aforementioned TCGA and DFCI/MSKCC studies, where tumor next-generation sequencing (NGS) was performed prior to any treatment. This decrease is consistent with prior ctDNA MRD studies in other solid tumor malignancies. TERT and TP53 were the most commonly altered genes detected in urine cfDNA across the 42 patient cohort (see e.g., FIG. 2D), consistent with the most commonly mutated genes in MIBC patients reported by TCGA and MSKCC. TERT promoter and TP53 mutations were detected in 24% ( 10/42) and 21% ( 9/42) of cases, respectively. uCAPPSeq was also performed on 15 healthy adults to query the specificity of the MRD gene panel variant data. Only 2 mutations, one in the PLEKHS1 intron and the other in ATM, were identified among these 15 healthy adults.

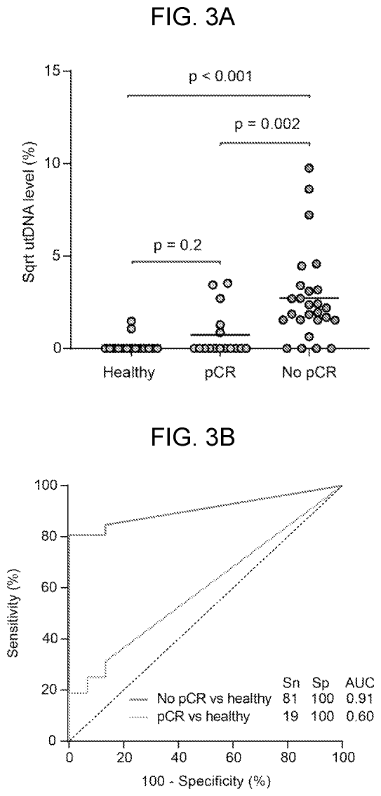

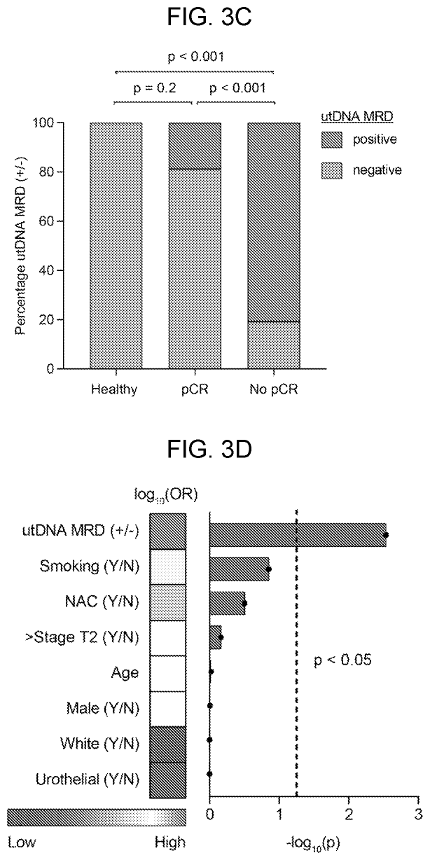

Moving forward in the analysis, a utDNA level was represented for each patient based on the non-silent mutation with the highest variant allele fraction. Comparing utDNA levels between the groups, patients with no pCR had a significantly higher median utDNA level compared to patients with pCR (4.3% versus 0%, p=0.002) and healthy adults (4.3% versus 0%, p<0.001) (see e.g., FIG. 3A). In contrast, there was no difference in the median utDNA levels between healthy adults and patients with pCR (0% versus 0%, p=0.2), implying that patients with pCR have similarly low biological background of urine variants as normal healthy adults. Variant data from the healthy adults was then used to determine the optimal utDNA level threshold that classifies localized bladder cancer patients from healthy adults. Based on an ROC curve, this threshold was determined to be 2.3%, which classified patients with no pCR from healthy adults with a sensitivity of 81% and specificity of 100% (AUC=0.91; 95% CI: 0.81 to 1.0) (see e.g., FIG. 3B). Therefore, patients with a utDNA level above or below this threshold were designated as utDNA MRD positive or negative, respectively.

The proportion of utDNA MRD-positive patients varied among patients with pCR, patients with no pCR, and healthy adults (see e.g., FIG. 3C). A total of 21 out of 26 (81%) patients with no pCR were utDNA MRD positive. Positive utDNA MRD detection was significantly associated with no pCR by Fisher exact test (p<0.001) with a sensitivity of 81% and specificity of 81%. Positive predictive value (PPV) and negative predictive value (NPV) were 88% ( 21/24) and 72% ( 13/18), respectively. A multivariate logistic regression analysis was performed including 8 covariates to predict the absence of pCR: utDNA MRD detection, smoking status, administration of neoadjuvant chemotherapy, AJCC 8 tumor stage, age (continuous variable), sex, ethnicity, and histology (see e.g., FIG. 3D). This further corroborated utDNA MRD detection as the sole predictor of pathologic response (p=0.003, odds ratio=54.2), which established a clear separation between patients with pCR and patients with no pCR (see e.g., TABLE 7).

TABLE 7
Multivariate logistic regression to predict the absence of
pathologic complete response.

| Variable | Odds ratio^{a} | p value^{a} |
|---|---|---|
| utDNA MRD^{b} (+/−) | 54.17 | 0.003 |
| Age | 1.00 | 0.945 |
| Smoking (yes/no) | 0.21 | 0.141 |
| Urothelial (yes/no) | 1.4E−07 | 0.997 |
| Sex (male/female) | 1.02 | 0.986 |
| Stage T3 or higher^{c} (yes/no) | 0.57 | 0.688 |
| Ethnicity (white/non-white) | 1.7E−09 | 0.995 |
| Neoadjuvant chemotherapy (yes/no) | 3.64 | 0.314 |

^{a}p values and odds ratio were calculated after multivariate logistic regression to predict the lack of pathologic complete response in bladder cancer patients (see Methods: Power and Statistical Analyses)
^{b}Urine tumor DNA minimal residual disease detection status was determined as described in the Methods: Power and Statistical Analyses.
^{c}Staging was performed at the time of pre-treatment TURBT using AJCC 8 criteria.
AJCC 8 = American Joint Committee on Cancer 8th edition
MRD = minimal residual disease
TURBT = transurethral resection of bladder tumor
utDNA = urine tumor DNA

Using utDNA MRD to predict pCR in a univariate logistic regression, leave-one-out cross-validation was also applied. Cross-validation revealed a highly significant accuracy of 81% (p=0.007), which suggests that the prediction model generalizes well to independent data.

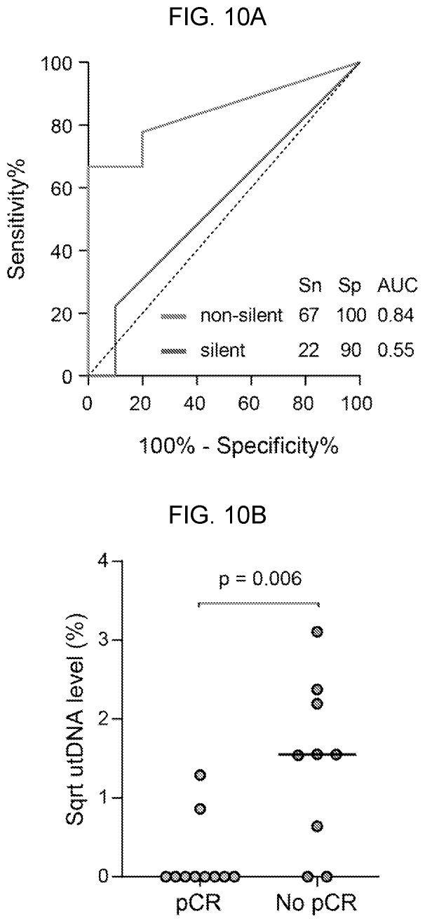

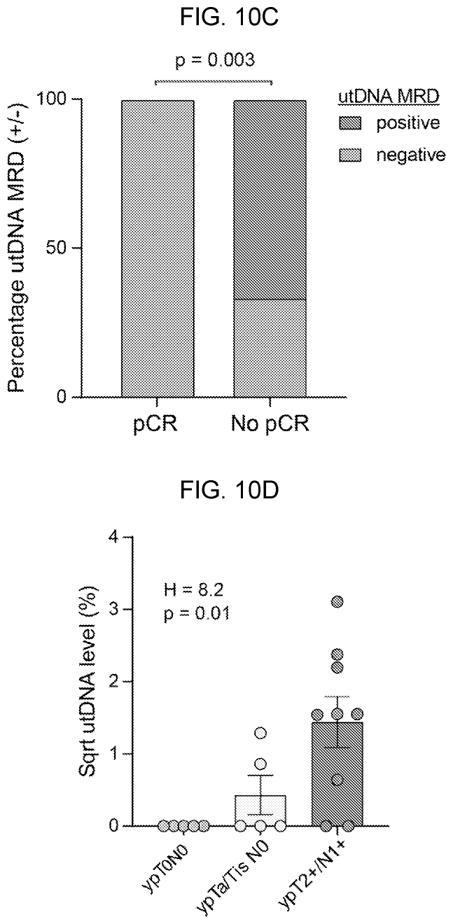

The association between pathologic response and utDNA remained highly significant when restricting the analysis to MIBC patients treated first with neoadjuvant chemotherapy. An ROC curve again showed that non-silent mutations (AUC=0.84; 95% CI: 0.65 to 1.0) distinguish pCR from no pCR with higher accuracy than silent mutations (AUC=0.55; 95% CI: 0.28 to 0.82) (see e.g., FIG. 10A). Moreover, patients with no pCR had a significantly higher utDNA level than patients with pCR (2.4% versus 0%, p=0.006) (see e.g., FIG. 10B). Using the same utDNA threshold described above for the full cohort, 67% of patients with no pCR who received neoadjuvant chemotherapy were utDNA MRD positive, while 100% of patients with pCR who received neoadjuvant chemotherapy were utDNA MRD negative (see e.g., FIG. 10C). This difference in the proportion of patients designated as utDNA MRD positive between each pathologic response group was once again highly significant by Fisher exact test (p=0.003). Notably, stratifying patients who received neoadjuvant chemotherapy by pathologic staging also showed a stepwise increase in detectable utDNA from ypTON0 to ypTa/Tis N0 patients (see e.g., FIG. 10D). Thus, a utDNA threshold trained to distinguish ypT0N0 from ypTa/Tis N0 disease has the potential to be even more granular for detecting residual disease after neoadjuvant chemotherapy in MIBC patients.

utDNA MRD as a Prognostic Biomarker

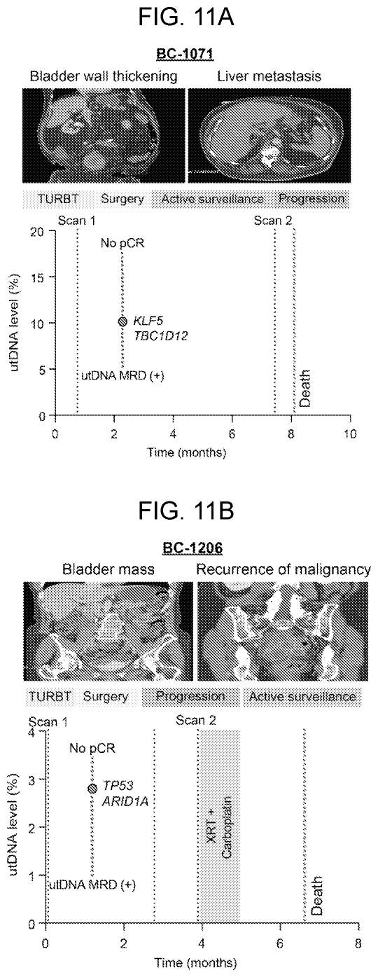

BC-1071 and BC-1206 are both MIBC patients who did not attain a pCR, and who, as expected, had positive utDNA MRD detection on preoperative urine sample collection. BC-1071 was a poor candidate for neoadjuvant chemotherapy and proceeded directly to radical cystectomy (see e.g., FIG. 11A). utDNA MRD was detected at a high level of 10.1% with nonsynonymous variation in the KLF5 coding region and a UTR mutation in TBC1D12.

Nonetheless, the oncology team deferred adjuvant therapy as the patient was determined to be a poor candidate. Unfortunately, this patient developed metastasis to the liver 5.3 months after surgery and passed away shortly thereafter. Similarly, BC-1206 proceeded directly to radical cystectomy after oncologists considered this patient to be a suboptimal neoadjuvant chemotherapy candidate (see e.g., FIG. 11B). utDNA MRD was detected at a level of 2.8% with a stop-gain mutation in TP53 and a nonsynonymous variation in the ARID1A coding region. Still, the patient elected to undergo active surveillance after surgery, but, unfortunately, developed progressive disease just 1.5 months later with peritoneal and bowel metastases and died 5.4 months thereafter.

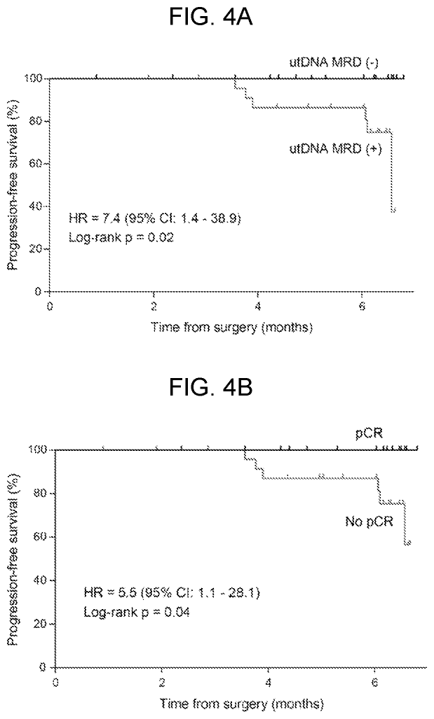

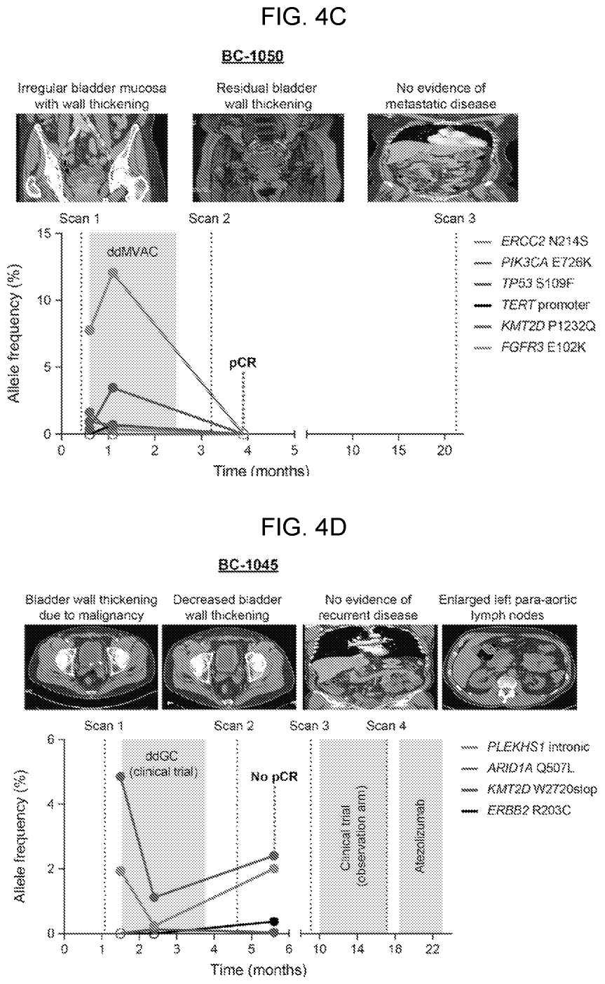

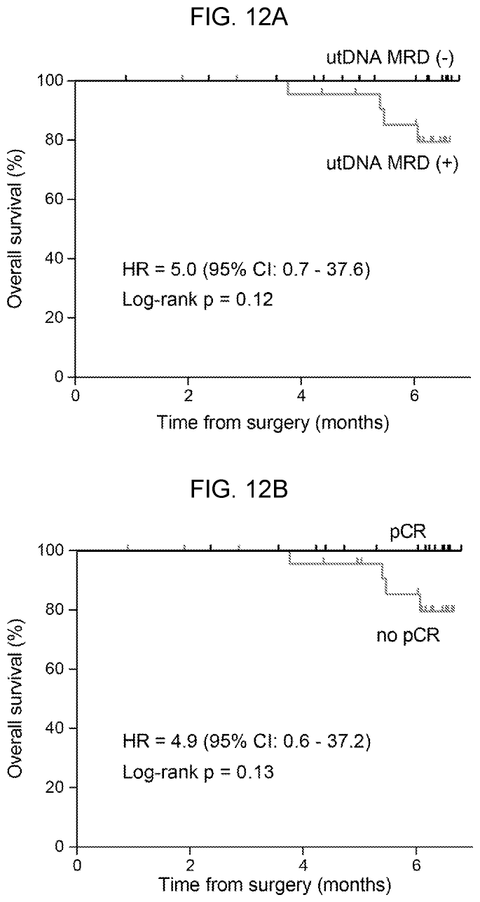

These clinical vignettes raised the question of how utDNA MRD detection compared to pathologic response in terms of predicting clinical outcomes, such as PFS and OS. Kaplan-Meier analysis of patients in the cohort was performed based on either utDNA MRD detection or pathologic response for PFS and OS. Six patients in the cohort recurred within 200 days with a median time to recurrence of 148 days. Notably, all 6 of these early-relapsed (early detection of disease recurrence or disease returning after curative-intent treatment)) patients were utDNA MRD positive and exhibited significantly worse PFS compared to utDNA MRD-negative patients (HR=7.4; 95% CI: 1.4 to 38.9; p=0.02) (see e.g., FIG. 4A). This effect size was comparable to the PFS difference between patients with pCR compared to no pCR (HR=5.5; 95% CI: 1.1 to 28.1; p=0.04) (see e.g., FIG. 4B), suggesting that utDNA MRD detection is on par with pCR measured by a board-certified genitourinary pathologist in predicting PFS for up 6 months after surgery. After 6 months, utDNA MRD detection may potentially be superior to pathologic response, but more data are required to confirm this. Furthermore, utDNA MRD detection (HR=5.0; 95% CI: 0.7 to 37.6; p=0.12) (see e.g., FIG. 12A) and pathologic response (HR=4.9; 95% CI: 0.6 to 37.2; p=0.13) (see e.g., FIG. 12B) corresponded to differences in OS, but analysis of OS was not powered for statistical significance.

Serial Tracking of utDNA Variants During Neoadjuvant Chemotherapy

utDNA MRD detection was also correlated with pathologic response for 2 patients (BC-1050 and BC-1045) who were treated with neoadjuvant chemotherapy and had multiple urine samples collected at different time points preceding radical cystectomy (see e.g., TABLE 8).

TABLE 8
Serial monitoring of urine tumor DNA on neoadjuvant chemotherapy.

| | | | | | | | Amino |
|---|---|---|---|---|---|---|---|
| Patient | | | | Mutation | Mutant | Reference | acid |
| ID | Gene | Chromosome | Position^{a} | type^{b} | allele | allele | change |
| BC-1045 | PLEKHS1 | chr10 | 115511590 | intronic | C | G | none |
| BC-1045 | ARID1A | chr1 | 27057812 | Non- | T | A | GLN > LYS |
| | | | | synonymous | | | |
| | | | | SNV | | | |
| BC-1045 | KMT2D | chr12 | 49433287 | stopgain | T | C | TRP > stop |
| BC-1045 | ERBB2 | chr17 | 37866098 | Non- | T | C | ARG > CYS |
| | | | | synonymous | | | |
| | | | | SNV | | | |
| BC-1050 | ERCC2 | chr19 | 45867687 | Non- | C | T | ASN > SER |
| | | | | synonymous | | | |
| | | | | SNV | | | |
| BC-1050 | PIK3CA | chr3 | 178938934 | Non- | A | G | GLU > LYS |
| | | | | synonymous | | | |
| | | | | SNV | | | |
| BC-1050 | TP53 | chr17 | 7577559 | Non- | A | G | SER > PHE |
| | | | | synonymous | | | |
| | | | | SNV | | | |
| BC-1050 | TERT | chr5 | 1295326 | upstream | T | C | none |
| BC-1050 | KMT2D | chr12 | 49443676 | Non- | T | G | PRO > GLN |
| | | | | synonymous | | | |
| | | | | SNV | | | |
| BC-1050 | FGFR3 | chr4 | 1801175 | Non- | A | G | GLU > LYS |
| | | | | synonymous | | | |
| | | | | SNV | | | |

| | | | |
|---|---|---|---|
| Amino | Pre-NAC* | On NAC* | Post-NAC/Pre-RC* |

| Patient | acid | Unique | VAF | Unique | VAF | Unique | VAF |
|---|---|---|---|---|---|---|---|
| ID | position | depth^{c} | (%)^{d} | depth^{c} | (%)^{d} | depth^{c} | (%)^{d} |
| BC-1045 | none | 1137 | 1.93 | 1261 | 0.24 | 1247 | 2.00 |
| BC-1045 | 507 | 4809 | 0.00 | 5413 | 0.13 | 5796 | 0.03 |
| BC-1045 | 2720 | 1030 | 4.85 | 984 | 1.12 | 1078 | 2.41 |
| BC-1045 | 173/188/203 | 2757 | 0.00 | 2640 | 0.00 | 3520 | 0.37 |
| BC-1050 | 214/238 | 2026 | 7.75 | 3426 | 12.05 | 629 | 0 |
| BC-1050 | 726 | 728 | 0.41 | 666 | 3.45 | 202 | 0 |
| BC-1050 | 82/109/202 | 1273 | 0.94 | 1867 | 0.64 | 590 | 0 |
| BC-1050 | none | 1044 | 0.00 | 1441 | 0.69 | 386 | 0 |
| BC-1050 | 1232 | 1486 | 1.62 | 1983 | 0.00 | 466 | 0 |
| BC-1050 | 102 | 1739 | 0.00 | 2524 | 0.36 | 550 | 0 |

*Samples were analyzed at 3 timepoints -- before neoadjuvant chemotherapy (“Pre-NAC”), during neoadjuvant chemotherapy (“On NAC”) and after neoadjuvant chemotherapy (“Post-NAC/Pre-RC”).
^{a}Genomic coordinates are per the GRCH37/hg19 genome assembly (see Methods: DNA Library Construction and Sequencing).
^{b}Mutational type was determined using ANNOVAR following uCAPP-Seq analysis (see Methods: Duplex-Supported Variant-Calling for utDNA MRD Analysis).
^{c}De-duplicated sequencing depth in urine cell-free DNA at the specified genomic position (see Methods: DNA Library Construction and Sequencing).
^{d}Variant allele fraction calculated as the percentage of mutant reads at the specified genomic position in urine cell-free DNA following uCAPP-Seq analysis (see Methods: DNA Library Construction and Sequencing).
chr = chromosome
NAC = neoadjuvant chemotherapy
RC = radical cystectomy
uCAPP-Seq = urine Cancer Personalized Profiling by Deep Sequencing
VAF = variant allele fraction

Serial urine samples were obtained from these 2 patients prior to starting neoadjuvant chemotherapy, during neoadjuvant chemotherapy, and on the day of radical cystectomy (MRD time point). All variants detected were monitored at each time point with CAPP-Seq. BC-1050 is a MIBC patient treated with 4 cycles of dose-dense methotrexate, vinblastine, doxorubicin, and cisplatin (see e.g., FIG. 4C). Surveillance imaging after neoadjuvant chemotherapy continued to denote minimal bladder wall thickening, which was equivocal for reflecting treatment response versus residual disease. utDNA MRD was not detected, however, suggesting a complete tumor response to neoadjuvant chemotherapy. The patient proceeded to radical cystectomy, where surgical pathology revealed pCR. The patient furthermore remained disease free on longer-term follow-up. Thus, both pCR and disease-free survival corroborated the negative utDNA MRD result.

Strikingly, additional utDNA analysis of serial urine samples preceding the MRD time point reflected changes in variant allele frequencies that were consistent with treatment response (see e.g., FIG. 4C). Prior to starting neoadjuvant chemotherapy, 4 non-silent mutations were detected with the highest being an ERCC2 nonsynonymous driver mutation, which has been associated with increased response to chemotherapy. Then, during chemotherapy (before starting the second cycle), half of these mutations, including this same ERCC2 mutation, showed increased allele frequencies by an average of 5-fold relative to the initial measurement. Two new mutations that were previously undetected before chemotherapy also emerged at this time point during chemotherapy. However, these mutations all decreased significantly and became undetectable at the MRD time point, reflecting a robust response to neoadjuvant chemotherapy consistent with the pCR result. Thus, in retrospect, this patient represents an intriguing candidate for future clinical trials testing utDNA as a means to personalize the duration of neoadjuvant chemotherapy and potentially optimize candidacy for bladder-sparing treatment.

BC-1045 is another MIBC patient who was treated with 5 cycles of dose-dense gemcitabine and cisplatin on a clinical trial, and the sixth cycle was held due to neuropathy and tinnitus (see e.g., FIG. 4D). Surveillance imaging after chemotherapy noted a decrease, but not a complete disappearance, of baseline bladder wall thickening. Imaging also never revealed suspicion for nodal involvement. Preoperative utDNA MRD was detected, however, with the highest mutant allele frequency being in KMT2D, which harbored a stop-gain mutation.

The patient went on to receive a radical cystectomy, and notably pathologic assessment revealed no residual tumor in the bladder, suggesting a pCR in the urothelium (see e.g., FIG. 4D). Nodal disease was identified in the surgical specimen, however, which had been missed in the 2 prior diagnostic imaging studies. On subsequent surveillance, the patient developed metastatic left para-aortic lymphadenopathy within a year of surgery, which necessitated immune checkpoint blockade. In this challenging diagnostic case where preoperative imaging and pathologic analysis of the urothelium could have been misleading, utDNA MRD analysis was able to correctly identify nodal disease involvement and correlated with early disease progression on follow-up. Thus, the presence of utDNA in this patient may have represented plasma-derived transrenal cfDNA originating from disease outside the urothelium. This suggests that the assay has the potential to detect nodal or distant disease in the absence of intravesical disease.

For BC-1045, utDNA analysis of serial urine samples was also performed before and during neoadjuvant chemotherapy (see e.g., FIG. 4D). Shortly after diagnosis and prior to any neoadjuvant chemotherapy, the patient's urine harbored non-silent mutations in PLEKHS1 and KMT2D, present at variant allele frequencies of 1.9% and 4.9%, respectively. Interestingly, both of these mutations decreased during neoadjuvant chemotherapy with their allele frequencies going down by an average of 6.2-fold, consistent with the partial treatment response seen on imaging following chemotherapy. However, utDNA analysis at the preoperative MRD time point revealed a subsequent increase in the allele frequencies of these 2 mutations by an average of 5.2-fold, reflecting the development of treatment resistance, further highlighted by the emergence of newly detected mutations in ARID1A and ERBB2 since chemotherapy was initiated. This patient represents a fascinating candidate for future clinical trials testing utDNA to personalize the regimentation of neoadjuvant and adjuvant systemic treatment based on serial utDNA analysis.

utDNA MRD Oncogenomics for Treatment Personalization

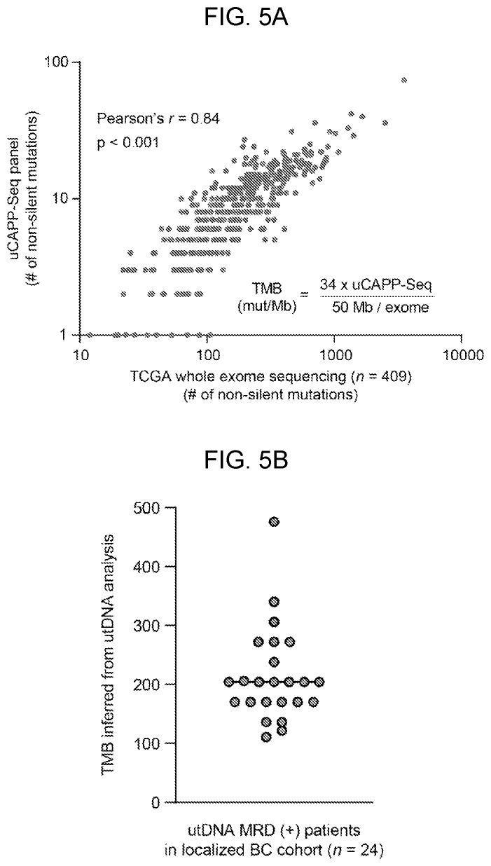

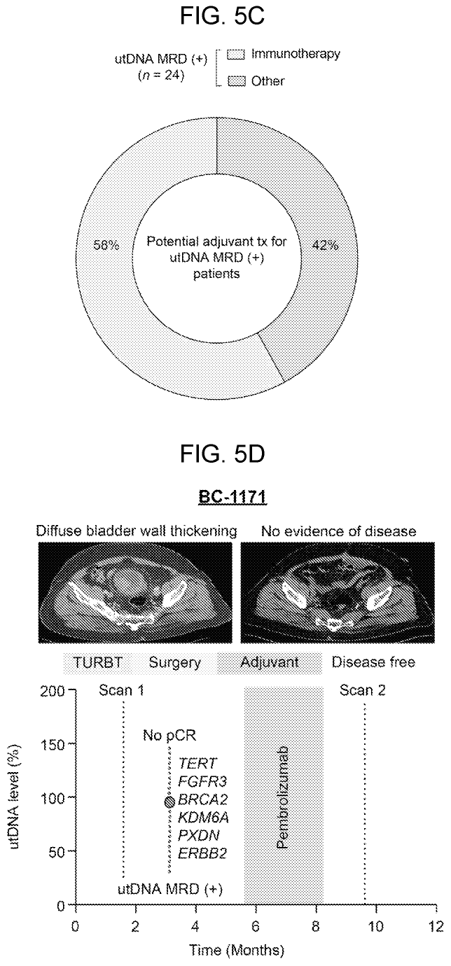

Although neoadjuvant immunotherapy has been approved for frontline administration in cisplatin-ineligible patients with metastatic bladder cancer, its use in the adjuvant setting is still being validated with several large clinical trials currently underway. It has previously been shown that TMB can be inferred from hybrid-capture cfDNA analysis in patients with non-small cell lung or colorectal cancer (see e.g., Chaudhuri et al. (2017) Cancer Discov. 7(12):1394:1403). To determine if similar methodology can be applied to bladder cancer, a linear regression model was generated based on whole exome sequencing of 409 MIBC tumors in TCGA and the corresponding non-silent mutational burden detected by an expanded hybrid-capture panel for assessing TMB (see e.g., FIG. 5A and TABLE 9).

TABLE 9
Genes fully or partially covered in TMB panel.

| | | | | | |
|---|---|---|---|---|---|
| ABCA6 | C16orf71 | COLGALT2 | ERBB3 | HCG4P5 | KCNH6 |
| ABCC5 | C1QTNF1 | COPB2 | ERBB4 | HCN1 | KCTD19 |
| ACAA1 | C3orf17 | COPG1 | ERCC2 | HECW1 | KDM3A |
| ACPP | C3orf20 | CPD | ERCC5 | HELZ | KDM6A |
| ACTL8 | C3orf58 | CPSF6 | FAM71A | HERC1 | KIAA0100 |
| ADAMTS12 | C3orf70 | CRB1 | FAM71F1 | HEXIM1 | KIAA0556 |
| ADAMTS16 | C9orf156 | CREBBP | FANCC | HHIP | KIAA1045 |
| ADRA1A | C9orf64 | CRTC2 | FANCM | HHLA2 | KLF5 |
| AHR | CACNA1A | CRYAB | FBXL7 | HIST1H1B | KLF6 |
| AKT1 | CACNA2D4 | CSN3 | FBXO42 | HIST1H1C | KMT2C |
| ALAS1 | CACNG4 | CSPG5 | FBXW7 | HIST1H1E | KMT2D |
| ALDH16A1 | CADM2 | CTNNA2 | FCRL3 | HIST1H2AG | KRAS |
| ALYREF | CARD11 | CTNNB1 | FERD3L | HIST1H2AK | KRIT1 |
| AMACR | CARD14 | CTNND2 | FETUB | HIST1H2BG | LAX1 |
| AMIGO3 | CBLB | CTTN | FFAR4 | HIST1H2BH | LHFPL5 |
| ANAPC11 | CCDC129 | CUBN | FGFR1 | HIST1H2BI | LILRA2 |
| ANKRD28 | CCDC173 | CUL1 | FGFR2 | HIST1H2BK | LILRB4 |
| ANO4 | CCDC36 | CYP7A1 | FGFR3 | HIST1H3B | LIPH |
| APOA4 | CCDC96 | DACH1 | FLRT2 | HIST1H3F | LPAR6 |
| AQP7 | CCND1 | DCAF12L2 | FOXA1 | HIST1H3H | LRGUK |
| ARHGEF16 | CCND3 | DCAF4L2 | FOXA3 | HIST1H3I | LRRC7 |
| ARID1A | CCNE1 | DDX42 | FOXQ1 | HIST1H4D | LRRK2 |
| ARID4A | CCR5 | DGCR8 | FURIN | HIST1H4I | LRRN1 |
| ASH1L | CD1D | DGKG | FZD6 | HIST1H4L | LRRTM1 |
| ASIC2 | CD5L | DHPS | GABRA1 | HLA-A | LRTM2 |
| ASXL2 | CD86 | DICER1 | GABRB1 | HLA-DQB2 | MAGI1 |
| ATM | CDC27 | DIDO1 | GABRG1 | HNRNPA2B1 | MAGI2 |
| ATP10A | CDH1 | DLC1 | GALNT8 | HOXA13 | MAP1LC3A |
| ATP8B4 | CDH10 | DLEC1 | GALR2 | HOXA7 | MAPK8 |
| ATR | CDH6 | DLGAP1 | GAREM | HOXB1 | MAPKAPK3 |
| ATXN2L | CDK12 | DLGAP5 | GATA3 | HOXB3 | MARCHF1 |
| AUTS2 | CDK6 | DNAH10 | GCNT2 | HOXB4 | MB21D2 |
| BCAS3 | CDKN1A | DNAH11 | GEMIN2 | HRAS | MCL1 |
| BCL2L1 | CDKN2A | DNAH5 | GHSR | HRH1 | MCM7 |
| BIRC6 | CDKN2B | DNTT | GLI3 | HSPB2 | MDM2 |
| BMS1 | CEP63 | DOPEY2 | GMPPB | HSPB2-C11orf52 | MED12 |
| BPIFB4 | CEP89 | DPYS | GNA13 | HTR1A | MED13 |
| BRAF | CFL1 | DSC1 | GNAT1 | HTRA2 | MET |
| BRCA1 | CHD2 | DTX1 | GNGT1 | ID1 | METTL3 |
| BRCA2 | CLASP2 | E2F3 | GOLGA1 | IDH1 | MGAT5B |
| BRINP3 | CLCNKB | EEF1B2 | GPC5 | IDH2 | MIOS |
| BRIP1 | CLIP1 | EFCAB5 | GPC6 | IKZF2 | MIR1306 |
| BRMS1 | CLVS2 | EGFR | GRIK3 | IRX1 | MIR4728 |
| BTG2 | CNKSR3 | EIF6 | GRM7 | ITGA3 | MLIP |
| C10orf90 | CNTN1 | ELF3 | GSS | ITIH3 | MMP25 |
| C11orf16 | CNTN6 | EMILIN2 | GTPBP8 | JAK2 | MMP9 |
| C11orf65 | CNTNAP1 | EP300 | GUCY1A3 | KARS | MOCS1 |
| C12orf43 | COL11A2 | EPHA3 | GYS1 | KCNA1 | MPHOSPH8 |
| C12orf66 | COL1A2 | EPHB4 | H1FOO | KCNB2 | MROH2B |
| C15orf52 | COL6A3 | ERBB2 | HARBI1 | KCND2 | MRPS22 |
| MS4A3 | PCDHGA5 | RBM47 | SUMF1 | WAC | |
| MTAP | PCDHGB1 | RBP4 | SYNE1 | WDR64 | |
| MTOR | PCDHGB2 | RECQL4 | SYNE2 | WNT7A | |
| MYC | PCDP1 | RECQL5 | SYT10 | XKR7 | |
| MYCL | PCF11 | RHOA | SYT15 | YAE1D1 | |
| MYF5 | PCK1 | RHOB | T | YAP1 | |
| MYH3 | PCSK6 | RHOU | TADA2B | YWHAZ | |
| MYH7 | PDE1C | RIPK4 | TBC1D12 | ZACN | |
| MYO3A | PDE3A | RIT2 | TBC1D16 | ZBTB22 | |
| NAB2 | PDE4DIP | RNASE3 | TBKBP1 | ZBTB45 | |
| NACA2 | PEG3 | RNF10 | TCFL5 | ZBTB7B | |
| NANOS3 | PGAP3 | RNF111 | TENM3 | ZFC3H1 | |
| NCKAP5 | PHACTR1 | RNF123 | TERT | ZFHX4 | |
| NCOR1 | PHLDA3 | RNF213 | TFEC | ZFP36L1 | |
| NDUFAF6 | PIGS | RNF216 | TFPI2 | ZFP36L2 | |
| NEURL4 | PIK3CA | RNF220 | TGIF2LX | ZFYVE1 | |
| NF1 | PKHD1 | ROBO2 | THAP2 | ZFYVE9 | |
| NFE2L2 | PKHD1L1 | RTN4RL2 | THSD7B | ZIC1 | |
| NKIRAS2 | PLAUR | RUSC1 | TIE1 | ZIM2 | |
| NKTR | PLEKHA7 | RXRA | TMCO2 | ZIM3 | |
| NKX2-2 | PLEKHS1 | RYR2 | TMCO4 | ZNF263 | |
| NLRP8 | POLDIP2 | S1PR1 | TMEM131 | ZNF276 | |
| NOM1 | POLE | SAMD4B | TMEM143 | ZNF277 | |
| NOS3 | POLN | SATB2 | TMEM200A | ZNF385D | |
| NOTCH4 | POM121L12 | SCAMP3 | TNXB | ZNF394 | |
| NR1D2 | POT1 | SCN4A | TOX4 | ZNF423 | |
| NRAS | PPARG | SEMA3D | TP53 | ZNF445 | |
| NRG3 | PPCS | SERPINA12 | TP53INP1 | ZNF511 | |
| NUTM1 | PPP2R1B | SF3B1 | TP63 | ZNF536 | |
| OTOF | PRICKLE1 | SGOL1 | TPRG1 | ZNF608 | |
| PAIP1 | PRKAA2 | SHOX2 | TRHR | ZNF620 | |
| PCDH10 | PRMT8 | SIGLEC8 | TRIM42 | ZNF671 | |
| PCDH17 | PSD2 | SLAMF1 | TRIM72 | ZNF703 | |
| PCDH9 | PSPH | SLC22A12 | TRPC4 | ZNF789 | |
| PCDHA1 | PTCHD2 | SLC32A1 | TRPS1 | ZSCAN22 | |
| PCDHA10 | PTEN | SLC38A2 | TSC1 | ZZEF1 | |
| PCDHA11 | PTPN22 | SLC6A15 | TSHZ3 | | |
| PCDHA12 | PTPN9 | SLC6A20 | TTC18 | | |
| PCDHA2 | PTPRK | SLTM | TTC19 | | |
| PCDHA3 | PTRF | SMARCA2 | TUBGCP2 | | |
| PCDHA4 | PVRL4 | SMARCA4 | TXNIP | | |
| PCDHA5 | PXDN | SNX22 | UBL4A | | |
| PCDHA6 | PXDNL | SNX32 | UPF2 | | |
| PCDHA7 | PYDC1 | SOX4 | UVSSA | | |
| PCDHA8 | RAB11FIP2 | SOX5 | VANGL1 | | |
| PCDHA9 | RAD51C | SPEG | VAT1 | | |
| PCDHGA1 | RARG | SPSB2 | VCAM1 | | |
| PCDHGA2 | RB1 | SPTAN1 | VSX1 | | |
| PCDHGA3 | RBM10 | STAG2 | VWA8 | | |
| PCDHGA4 | RBM26 | SUCNR1 | VWF | | |

TMB = tumor mutational burden

From this correlation (Pearson's r=0.84, p<0.001), an equation was derived to infer exome-wide mutational burden from non-silent mutations in utDNA detected by the TMB panel. Using this equation to infer TMB among utDNA MRD-positive patients in the cohort (n=24), the median TMB was 204 (range of 111 to 476) non-silent mutations per exome (see e.g., FIG. 5B and TABLE 10).

TABLE 10
Urine-inferred tumor mutational burden for utDNA MRD
positive patients.

| Patient ID | iTMB* |
|---|---|
| BC-1045 | 340 |
| BC-1058 | 136 |
| BC-1062 | 170 |
| BC-1069 | 204 |
| BC-1071 | 170 |
| BC-1076 | 306 |
| BC-1077 | 170 |
| BC-1083 | 476 |
| BC-1085 | 272 |
| BC-1088 | 170 |
| BC-1090 | 204 |
| BC-1096 | 272 |
| BC-1104 | 111 |
| BC-1111 | 204 |
| BC-1133 | 122 |
| BC-1135 | 136 |
| BC-1171 | 204 |
| BC-1174 | 204 |
| BC-1182 | 170 |
| BC-1186 | 238 |
| BC-1188 | 272 |
| BC-1202 | 206 |
| BC-1206 | 170 |
| BC-1209 | 204 |

*Exome-wide iTMB interpolated from the uCAPP-Seq results for utDNA MRD (+) patients using the TMB sequencing panel (TABLE 3) as described in the Methods: Inferring TMB from utDNA Analysis.
iTMB = inferred tumor mutational burden
uCAPP-Seq = urine Cancer Personalized Profiling by Deep Sequencing

High TMB is predictive of response to immune checkpoint blockade based on data from multiple studies in lung and bladder cancers. A recent retrospective study of 139 patients with advanced, unresectable urothelial carcinoma receiving nivolumab on a clinical trial (CheckMate 275) revealed that TMB categorization by tertiles, with the highest TMB tertile defined as 170 missense mutations or more, was significantly associated with improved objective response rate (ORR), PFS, and OS. Given the prognostic potential of this high TMB cutoff, patients with an inferred TMB greater than 170 non-silent mutations per exome may be classified as high TMB. Based on this cutoff, 58% of utDNA MRD-positive patients in the cohort with high urine-inferred TMB could potentially be identified as candidates for adjuvant immunotherapy (see e.g., FIG. 5C), thus facilitating the personalization of immune checkpoint blockade in the adjuvant setting for MIBC.

The cohort was also queried for potentially actionable mutations based on utDNA analysis, which revealed that 13% of utDNA MRD-positive patients also harbor mutations that have been shown to predict response to a drug in either bladder cancer or another solid tumor malignancy type (see e.g., TABLE 6). Potentially actionable mutations that were identified include an ERCC2 nonsynonymous mutation that is indexed in both the Catalogue of Somatic Mutations in Cancer (COSMIC) and OncoKB and correlates strongly with increased sensitivity to cisplatin-based chemotherapy. Other potentially actionable mutations identified were nonsynonymous mutations in PIK3CA and TSC1, also annotated in both COSMIC and OncoKB, and shown to be clinically targetable in breast cancer through PI3K inhibition and in central nervous system cancers with mTOR inhibition, respectively.

The remaining 42% of utDNA MRD-positive patients in the cohort harbored mutations that were neither known to be actionable nor present at elevated burdens. These MRD-positive patients might be appropriate candidates for other types of adjuvant therapy to ablate residual disease, such as chemotherapy or radiation therapy. Overall, the paradigm described in FIG. 5C could thus form the basis for future clinical trials utilizing utDNA-based personalization of adjuvant treatment for MIBC patients harboring MRD.

In this regard, 2 utDNA MRD-positive patients with high urine-inferred TMB were identified in the cohort who received immunotherapy on a clinical trial. BC-1171 was randomized to adjuvant treatment with pembrolizumab after radical cystectomy (see e.g., FIG. 5D). This patient harbored an inferred TMB of 204 mutations per exome, thus classifying this patient as high TMB using the criteria. This patient has shown no evidence of recurrence or progression since surgery, which is consistent with the prediction based on the proposed paradigm. BC-1045, who was discussed previously (see e.g., FIG. 4D), was randomized to the observation arm of this same trial (versus pembrolizumab). The patient was diagnosed with disease progression 10.2 months following surgery and thus enrolled onto a different trial, where they are receiving treatment with single-agent atezolizumab. With an elevated urine-inferred TMB of 340 mutations per exome at the preoperative MRD time point, this patient has achieved a radiographic complete response in the involved lymph nodes on atezolizumab. It is tempting to speculate that this patient with high utDNA-inferred TMB may have averted disease relapse altogether if offered immunotherapy shortly after the MRD time point.

Discussion

Herein is described a cohort study that demonstrates the significant correlation between utDNA MRD detected by uCAPP-Seq prior to curative-intent radical cystectomy and pathologic response determined by analysis of the surgical specimen. This was demonstrated by utDNA analysis with a focused gene panel of non-silent mutations in consensus driver genes, which indicates that association with pathologic response is strongly linked to the detection of mutations that impact the phenotype of bladder cancer and are less likely to be confounded by background field effect. As expected, the most commonly mutated genes detected in urine cfDNA in the cohort were TERT and TP53, consistent with the results of larger tumor sequencing studies. The approach described herein also demonstrates an 85% level of concordance among mutations detected between tumor and utDNA for the subset of patients with tumor tissue available for analysis. Of note, the MRD detection approach only utilizes mutations with duplex support, which facilitates the low limit of detection necessary for MRD detection. It also does not require prior sequencing of tumor tissue, which is practically useful and biologically can help avoid confounding of results due to geographic tumor heterogeneity.

Targeted NGS has previously been applied to detect early-stage bladder cancer in reasonably large cohorts using methods including UroSEEK and uCAPP-Seq. utMeMa, a methylation-based assessment of utDNA, has also shown feasibility in the MRD setting. However, these previous urine-based sequencing approaches were either largely restricted to or exclusively studied in NMIBC and did not corroborate residual disease detection with a radical cystectomy specimen, which is currently the most accurate assessment of residual disease after standard of care treatment for MIBC. Moreover, there are several key differences in the biology and prognosis of NMIBC and MIBC, as the latter often arises via progression of urothelial dysplasia to muscle-invasive disease due to the accumulation of genomic alterations. This is potentially related to an exacerbated field cancerization effect that may increase the risk of advanced disease and is associated with higher levels of nonspecific genomic alterations. These distinctions require important technological advancements that optimize variant calling for MIBC.

In this study, MIBC was focused on with every patient being treated with radical cystectomy as part of their clinical management. Moreover, given that the cohort was predominantly composed of muscle-invasive patients, a high percentage received neoadjuvant chemotherapy. In this regard, state-of-the-art targeted deep sequencing was applied using uCAPP-Seq technology to identify duplex-supported variants and MRD was analyzed in an important population not thoroughly assessed previously. Moreover, the findings were correlated with gold standard clinical outcomes including pathologic response, PFS, and OS.

The data shown herein suggest that utDNA MRD detection can accurately predict pathologic response in MIBC patients. Predicting pathologic response in this fashion may enable personalization of treatment decisions for MIBC patients in the future. For example, utDNA MRD detection status may help inform bladder-sparing treatment, potentially enabling clinicians to forego radical cystectomy altogether in selected patients found to be MRD negative. Conversely, patients found to be utDNA MRD positive may be prioritized for further treatment such as targeted or immune therapy, which may be personalized based on the oncogenomic features learned from utDNA analysis. Notably, any activating mutations in FGFR3 were not detected in the cohort, which have been shown to predict responses to tyrosine kinase inhibitors in bladder cancer. For example, erdafitinib is an FGFR inhibitor that is FDA approved for use in patients with locally advanced or metastatic bladder cancer. Therefore, in addition to the mutations highlighted in the cohort, utDNA may have the potential to enable personalized systemic treatment with erdafitinib for patients harboring FGFR mutations in the near future.

It was also shown herein through clinical vignettes that monitoring of utDNA before, during, and after neoadjuvant chemotherapy may be used to temporally monitor treatment response versus the emergence of resistance. Using this type of temporally high-resolution utDNA analysis, the strength and regimentation of neoadjuvant treatment may be further optimized, along with the consideration and timing of radical cystectomy. utDNA may also be used as an adjunct to other emerging modalities in this regard, such as VI-RADS, a newly developed MRI-based staging criteria, to more definitively predict treatment response and clinical outcomes in the future.

Limitations of the study include that utDNA analysis was performed considering only SNVs, which are the most predominant genomic alterations observed in bladder cancer, a cancer type with among the highest SNV burdens overall. Additionally, it may be desired to further validate the findings presented herein before clinical implementation. As the tumor genomic characterization of MIBC broadens in the future, the sensitivity of the assay will improve. Future work to improve the sensitivity of residual disease detection may also explore the combination of utDNA analysis with clinical evaluation (i.e., cystoscopy), as well as incorporating other genomic alterations into the method (e.g., copy number variations, insertions/deletions, and fusions).

It is also noted that all utDNA testing in this study was performed prior to radical cystectomy, which enabled assessment of the ability of utDNA MRD to predict pathologic response. However, utDNA analysis at this time point makes it challenging to assess potential subsequent prognostic implications, since the risk of disease progression or death is typically diminished by curative-intent surgical intervention. As a result, a utDNA MRD-positive patient at elevated risk of disease progression could have their MRD fully addressed via surgery, and, thus, could remain disease free in the Kaplan-Meier analysis. Even with this limitation, however, a significant difference in PFS and likely a difference in OS was observed. An ongoing prospective clinical trial utilizing a multi-institutional cohort through the Alliance study will aim to further validate the ability of utDNA to predict complete response and survival outcomes. Future work may also include studying a cohort of healthy smokers in order to examine if utDNA can be used as a screening tool for bladder cancer.

Finally, it has been shown that mutations in DNA damage repair (DDR) genes, such as ERCC2, are predictive of chemotherapeutic response. This is consistent with the observation of an overall decrease in variant allele frequencies in serial urine samples collected during and after neoadjuvant chemotherapy for patient BC-1050, who harbored a nonsynonymous ERCC2 mutation. Still, the association between DDR mutations and chemotherapeutic response was not assessed in the cohort as a whole because these mutations at the MRD time point are mostly undetectable after chemotherapy is complete, as was the case for BC-1050. To increase the number of mutations detected in DNA damage repair genes, more patients may be enrolled or more urine samples may be collected and analyzed prior to neoadjuvant chemotherapy.

In conclusion, shown herein is that utDNA MRD detection with uCAPP-Seq classifies pathologic response with high sensitivity and specificity in a cohort comprised predominantly of MIBC. This approach has the potential to facilitate more personalized treatment interventions for MIBC in the future, such as curative-intent bladder-sparing approaches, adjuvant treatment with immune checkpoint blockade, and targeted systemic therapy administration.

Methods

Study Design

The cohort study was designed to determine whether utDNA on the day of radical cystectomy correlates with pathologic response and detects MRD in patients with localized bladder cancer. Thus, 48 patients were enrolled with localized bladder cancer undergoing curative-intent radical cystectomy and 27 healthy adults at Washington University from April 2019 to November 2020 (see e.g., FIG. 1). Patients and healthy adults were enrolled onto institutionalized human research protocols approved by the Human Research Protection Office at Washington University and provided written informed consent to the collection and molecular analysis of biological specimens (urine, blood, and/or tumor tissue) in accordance with the Declaration of Helsinki. Paired urine and blood samples acquired on the day of radical cystectomy from 42 consented patients were analyzed and profiled (see e.g., TABLE 2). Patients were excluded if they had a blood sample without urine, a urine sample without blood, or a history of immunosuppression for a solid organ transplant, which has been shown to confound the detection of tumor DNA. In 2 patients, serial urine samples were additionally obtained prior to and during neoadjuvant chemotherapy. Patients continued clinical follow-up after radical cystectomy as per the standard of care, and the managing clinicians were blinded to the utDNA data. Paired urine and blood samples were also collected from 15 healthy adults for variant calling analysis. Urine samples without blood were collected from another 12 healthy adults and used to reduce stereotypical base substitution errors for the serial mutation tracking and TMB analyses.

Eligibility Criteria

Patients were required to have a histologic or cytologic diagnosis of bladder cancer amenable for radical cystectomy. MIBC was defined as invasive urothelial carcinoma staged at least T2 or higher (i.e., invasion of the detrusor muscle) found on transurethral resection of bladder tumor or radiographically determined by a board-certified urologic oncologist and/or medical oncologist. In some equivocal cases, a multidisciplinary tumor board assessment was required to determine a case for surgical resection. The primary bladder cancer could have been previously treated with intravesical agents such as Bacillus Calmette-Guerin (BCG), valrubicin, or gemcitabine. Some cases of T1 disease were also allowed into the study. The decision to proceed with cystectomy in cases of T1 disease was made by the patient and the treating physician, often in collaboration with a multidisciplinary tumor board. These cases were resistant to prior intravesical chemotherapy and/or considered at high risk for progression based on pathological features. Any bladder cancer patient with an indication for radical cystectomy was included in the study in order to extend the application of predicting pathologic response based on utDNA to a broader, real-world patient population that could potentially benefit from bladder-sparing treatment. Patients with any other known active cancer diagnoses other than bladder cancer were excluded from enrollment. Electronic medical records were reviewed for each patient to ensure that they met the eligibility criteria for this study.

Pathologic Response Assessment

All surgical samples were sampled consistently following standard collection, handling, and submission procedures. The resected surgical specimens (e.g., bladder, prostate, and pelvic lymph nodes) were submitted to the Pathology Department at Washington University for review by a genitourinary surgical pathologist. Based on the eligibility criteria used in an enrolling multi-institutional Alliance trial on nonmetastatic MIBC, pCR was defined as stages TO, Tis, and/or Ta with no involved lymph nodes in the surgical specimen. This is consistent with the definition used in other solid tumor malignancies as well, including breast cancer. An absence of pCR was defined as stages T1 to T4 or evidence of nodal/metastatic disease in the surgical specimen based on the American Joint Committee on Cancer (AJCC) 8th edition criteria.

Urine Cell-Free DNA Stability in the Presence of EDTA

Multiple urine samples from a healthy adult were used to test the stability of urine cell-free DNA (cfDNA) in the presence of EDTA (see e.g., FIG. 6). Pooled urine from this individual was aliquoted into 4 pairs of two 10-mL replicates, each pair testing urine cfDNA stability at different time points (0, 1, 3, and 7 days), with or without EDTA. Thus, 10 mM EDTA was added to one aliquot, and an equal amount of nuclease-free water was added to the other aliquot. The urine samples were then stored at room temperature (approximately 20° C.) for these different lengths of times. After each time point, the urine was centrifuged, and the supernatant was transferred to fresh tubes and stored at −80° C. Urine cfDNA was isolated from the stored supernatant. Urine cfDNA was purified further by AMPure XP (Beckman Coulter Life Sciences, Indianapolis, Indiana, USA) magnetic bead-based size selection. Genomic DNA was removed using 0.6× concentration of beads, followed by 1.8× concentration of beads to capture the remaining urine cfDNA. Isolated urine cfDNA was analyzed by electropherogram obtained using an Agilent 2100 Bioanalyzer (Agilent Technologies, Santa Clara, California, USA).

Comparison of Urine Cell-Free DNA Fragmentation with in Silico Size Selection

Two different methodologies were compared for optimizing the detection of variants prior to urine cfDNA library preparation. Urine cfDNA was isolated with Q-sepharose from 4 MIBC patients, as described herein, and processed using 1 of 2 methodologies: standard whole urine cfDNA fragmentation or in vitro size selection (see e.g., FIG. 7). For the fragmentation method, urine cfDNA was acoustically sheared to approximately 200-bp fragments using a LE220 focused ultrasonicator (Covaris, Woburn, Massachusetts, USA). For the in vitro size selection protocol, AMPure XP magnetic beads were used to enrich cfDNA fragments ranging from 70 to 450 bp before sequencing. Following each method, sequencing libraries were prepared per the uCAPP-Seq protocol. The median deduplicated depth (see e.g., FIG. 8A), number of non-silent mutations detected (see e.g., FIG. 8B), and variant allele fraction levels (see e.g., FIG. 8C) were compared among the common non-silent mutations found by both methodologies. Only variants with duplex support were considered in this analysis.

Biological Specimen Collection and Processing

Urine samples, ranging in volume between 22 and 90 mL, from 42 localized bladder cancer patients were collected in sterile vials containing 2 mL of 0.5M EDTA in the preoperative setting. Samples were centrifuged at 2,000 g for 10 minutes at room temperature (approximately 20° C.). After centrifugation, supernatant was transferred to a fresh tube and then stored at −80° C. Along with urine, patient blood was collected in K2-EDTA Vacutainer tubes (Becton Dickinson, Franklin Lakes, New Jersey, USA), from which germline plasma-depleted whole blood was collected and stored at −80° C. as previously described (see e.g., Chaudhuri et al. (2017) Cancer Discov. 7(12):1394-403). Tumor tissue was also available for sequencing from 5 patients with no antecedent systemic therapy. Tumor tissue was acquired either from the radical cystectomy or transurethral resection of bladder tumor and was either stored fresh-frozen or processed as formalin-fixed paraffin-embedded samples.

Urine Cell-Free DNA Isolation and Quantification

Urine cfDNA was prepared from 22 to 90 mL of urine with Q-sepharose resin slurry (GE Healthcare, Chicago, Illinois, USA) at a ratio of 10 μL slurry per mL of urine and mixed as previously described (see e g., Dudley et al. (2019) Clin Cancer Res. 9(4):500-9 and Shekhtman et al. (2009) Clin Chem. 55(4):723-9). After 30 minutes, the urine/resin mixture was centrifuged for 10 minutes at 1,800 g. The supernatant was discarded, and resin was washed twice with 0.3M LiCl/10 mM sodium acetate (pH 5.5), applying 2 mL per 100-μL resin. The resin was then transferred to a Micro Bio-Spin column (Bio-Rad, Hercules, California, USA), and the bound material was eluted by adding 3 separate 670-μL aliquots of 2MLiCl/10 mM sodium acetate (pH 5.5). Next, the eluates were combined in 70% ethanol and passed over a QIAquick column (Qiagen, Hilden, Germany). The column was washed with 5 mL of 2 M LiCl in 70% ethanol, followed by 5 mL of 75 mM potassium acetate (pH 5.5) in 80% ethanol. Residual liquid was removed by centrifuging the columns at 20,000 g for 3 minutes. Finally, bound DNA was eluted into 50 μL of nuclease-free water or 10 mM Tris-Cl (pH 8.5). DNA concentrations were measured using a Qubit dsDNA HS Assay Kit and Qubit 4.0 Fluorometer (Thermo Fisher Scientific, Waltham, Massachusetts, USA).

Germline and Tumor DNA Isolation and Quantification

The QIAamp DNA Micro Kit (Qiagen) was employed to extract germline DNA from 100 μL of plasma-depleted whole blood or tumor DNA from freshly frozen tumor tissue. The AllPrep DNA/RNA FFPE Kit (Qiagen) was used to extract DNA from one tumor-embedded paraffin block according to the manufacturer's instructions. DNA was then quantified using the Qubit assay per the manufacturer's recommendations (Thermo Fisher Scientific).

DNA Library Construction and Sequencing

Urine cfDNA was fragmented to approximately 200-bp size, while germline DNA and tumor DNA were sheared to approximately 250-bp size prior to library preparation using a LE220 Focused Ultrasonicator (Covaris). All urine cfDNA and germline DNA libraries were prepared using the Kapa HyperPrep Kit with custom sequencing adapters containing demultiplexing, deduplicating, and duplex barcodes. Minimum inputs of 32 ng were used for each library preparation reaction. Targeted hybrid-capture was performed per the uCAPP-Seq protocol with the following modifications: For urine CfDNA, 3 or 4 samples were captured with the custom bladder cancer selector panel (see e.g., TABLE 3 and TABLE 9), while 6-plex captures and 12-plex captures were performed for tumor and germline samples, respectively. Libraries were sequenced on an Illumina HiSeq 4000 with 2×150 bp paired-end reads. Sequencing quality control metrics including deduplicated depth, on-target rate, fragment size, and duplex recovery rate were reviewed (see e.g., TABLE 11-TABLE 13).

TABLE 11
Sequencing metrics of urine cell-free DNA for all patients.

| | | | | | | Median | |
|---|---|---|---|---|---|---|---|
| | | | On- | | Median | fragment | |
| Patient | Time- | NGS | target | Median | unique | length | Duplex |
| ID | point^{a} | reads^{b} | rate^{c} | depth^{d} | depth^{e} | (bp)^{f} | rate^{g} |
| BC-1045 | 1 | 86044908 | 66% | 12896 | 1672 | 211 | 14% |
| BC-1045 | 2 | 36433873 | 61% | 5600 | 1650 | 205 | 13% |
| BC-1045 | 3 | 36212756 | 51% | 4741 | 1705 | 222 | 11% |
| BC-1050 | 1 | 112448596 | 45% | 12047 | 416 | 197 | 19% |
| BC-1050 | 2 | 42308799 | 62% | 6301 | 1855 | 203 | 12% |
| BC-1050 | 3 | 47157779 | 47% | 5211 | 1540 | 195 | 9% |
| BC-1058 | 3 | 80082917 | 36% | 6964 | 803 | 199 | 22% |
| BC-1062 | 3 | 67273563 | 63% | 10779 | 1426 | 195 | 20% |
| BC-1063 | 3 | 59802599 | 59% | 6434 | 489 | 186 | 24% |
| BC-1064 | 3 | 114778624 | 45% | 13737 | 487 | 194 | 19% |
| BC-1065 | 3 | 39517627 | 36% | 3800 | 942 | 227 | 8% |
| BC-1069 | 3 | 53041349 | 32% | 4562 | 1068 | 198 | 8% |
| BC-1071 | 3 | 109814511 | 45% | 7547 | 1298 | 206 | 10% |
| BC-1076 | 3 | 69313200 | 64% | 6921 | 492 | 218 | 11% |
| BC-1077 | 3 | 84762979 | 66% | 12886 | 485 | 215 | 25% |
| BC-1083 | 3 | 131505614 | 54% | 15722 | 1458 | 190 | 13% |
| BC-1085 | 3 | 111290583 | 46% | 13793 | 811 | 220 | 10% |
| BC-1088 | 3 | 51991502 | 37% | 4383 | 912 | 184 | 15% |
| BC-1090 | 3 | 93625053 | 48% | 12061 | 558 | 223 | 17% |
| BC-1096 | 3 | 165042082 | 44% | 13953 | 639 | 189 | 6% |
| BC-1104 | 3 | 63724347 | 35% | 5805 | 483 | 198 | 29% |
| BC-1105 | 3 | 77435537 | 66% | 11461 | 459 | 202 | 29% |
| BC-1108 | 3 | 128660774 | 40% | 13423 | 900 | 197 | 4% |
| BC-1109 | 3 | 114256260 | 42% | 11524 | 568 | 196 | 11% |
| BC-1111 | 3 | 95443086 | 22% | 5256 | 806 | 194 | 24% |
| BC-1116 | 3 | 137313246 | 45% | 14536 | 843 | 203 | 6% |
| BC-1132 | 3 | 106717842 | 47% | 13667 | 753 | 221 | 15% |
| BC-1133 | 3 | 53052535 | 35% | 4796 | 690 | 198 | 29% |
| BC-1135 | 3 | 75073502 | 39% | 6987 | 426 | 224 | 30% |
| BC-1140 | 3 | 51949517 | 62% | 8517 | 430 | 216 | 30% |
| BC-1147 | 3 | 44255080 | 36% | 3878 | 776 | 193 | 19% |
| BC-1171 | 3 | 67617578 | 68% | 9606 | 1487 | 205 | 16% |
| BC-1174 | 3 | 74419432 | 38% | 7301 | 1083 | 225 | 23% |
| BC-1182 | 3 | 60706405 | 67% | 8902 | 684 | 200 | 30% |
| BC-1185 | 3 | 73723253 | 46% | 8165 | 1027 | 230 | 25% |
| BC-1186 | 3 | 59438216 | 45% | 6491 | 989 | 230 | 18% |
| BC-1188 | 3 | 40536268 | 40% | 3803 | 761 | 189 | 20% |
| BC-1196 | 3 | 53238995 | 22% | 3077 | 828 | 205 | 20% |
| BC-1197 | 3 | 50491235 | 23% | 3165 | 499 | 191 | 31% |
| BC-1198 | 3 | 63807796 | 62% | 9937 | 1496 | 208 | 23% |
| BC-1202 | 3 | 53258914 | 60% | 7863 | 758 | 211 | 26% |
| BC-1203 | 3 | 62150154 | 15% | 2346 | 714 | 195 | 15% |
| BC-1205 | 3 | 52853500 | 23% | 3091 | 834 | 201 | 22% |
| BC-1206 | 3 | 76458550 | 60% | 10843 | 1665 | 187 | 15% |
| BC-1207 | 3 | 43917622 | 16% | 1832 | 460 | 198 | 21% |
| BC-1209 | 3 | 61907852 | 15% | 2285 | 830 | 191 | 6% |

^{a}Timepoints when urine was collected for analysis: 1 is prior to neoadjuvant chemotherapy, 2 is during neoadjuvant chemotherapy, and 3 is just prior to radical cystectomy (see FIG. S1, Methods: Study Design, and Methods: utDNA Monitoring during Neoadjuvant Chemotherapy).
^{b}Number of total sequencing reads per sample after demultiplexing (see Methods: DNA Library Construction and Sequencing).
^{c}Percentage of quality-control-passed reads overlapping with the targeted hybrid-capture panel (see Methods: DNA Library Construction and Sequencing).
^{d}Median non-deduplicated sequencing depth after uCAPP-Seq (see Methods: DNA Library Construction and Sequencing).
^{e}Median deduplicated sequencing depth after uCAPP-Seq (see Methods: DNA Library Construction and Sequencing).
^{f}Median length of sonicated urine cell-free DNA fragments, as determined by uCAPP-Seq (see Methods: DNA Library Construction and Sequencing).
^{g}Percentage of deduplicated reads with duplex support as determined by uCAPP-Seq (see Methods: DNA Library Construction and Sequencing).
bp = base pairs
NGS = next-generation sequencing
uCAPP-Seq = urine Cancer Personalized Profiling by Deep Sequencing

TABLE 12
Sequencing metrics of urine cell-free DNA in healthy adults.

| | | | | | Median | |
|---|---|---|---|---|---|---|
| | | On- | | Median | fragment | |
| Patient | NGS | target | Median | unique | length | Duplex |
| ID* | reads^{a} | rate^{b} | depth^{c} | depth^{a} | (bp)^{e} | rate^{f} |
| 104 | 58916269 | 34% | 5247 | 947 | 220 | 24% |
| 106 | 62622449 | 43% | 6751 | 1110 | 191 | 21% |
| 130 | 54397548 | 26% | 3574 | 744 | 205 | 23% |
| 66 | 67061482 | 29% | 4677 | 627 | 186 | 25% |
| 67 | 77252438 | 29% | 5435 | 744 | 200 | 26% |
| 69 | 40188624 | 65% | 6148 | 278 | 187 | 26% |
| 70 | 62489901 | 31% | 4948 | 303 | 214 | 25% |
| 71 | 70061717 | 30% | 5317 | 993 | 211 | 22% |
| 72 | 48514767 | 62% | 7583 | 859 | 216 | 25% |
| 80 | 59001966 | 33% | 5010 | 1098 | 204 | 21% |
| 89 | 66212013 | 65% | 10866 | 711 | 206 | 24% |
| 92 | 57561920 | 44% | 6365 | 1090 | 192 | 23% |
| 94 | 67169795 | 34% | 5655 | 907 | 213 | 25% |
| 95 | 60919188 | 66% | 10188 | 563 | 224 | 25% |
| 98 | 63037934 | 32% | 5164 | 1013 | 200 | 22% |
| 116 | 70083256 | 50% | 9049 | 1242 | 212 | 24% |
| 117 | 64024880 | 49% | 8421 | 976 | 234 | 25% |
| 118 | 64306895 | 68% | 11073 | 1058 | 207 | 24% |
| 119 | 54997915 | 69% | 10145 | 733 | 199 | 28% |
| 120 | 47627231 | 62% | 7678 | 411 | 211 | 27% |
| 121 | 60542526 | 63% | 10212 | 786 | 187 | 26% |
| 122 | 61917418 | 46% | 7472 | 858 | 199 | 26% |
| 123 | 55229722 | 68% | 10061 | 913 | 212 | 27% |
| 124 | 59120740 | 68% | 10843 | 858 | 205 | 28% |
| 125 | 53722928 | 62% | 8609 | 701 | 198 | 25% |
| 126 | 52047196 | 62% | 8509 | 774 | 210 | 28% |
| 127 | 66200441 | 49% | 8575 | 565 | 188 | 30% |

^{a}Number of total sequencing reads per sample after demultiplexing (see Methods: DNA Library Construction and Sequencing).
^{b}Percentage of quality-control-passed reads overlapping with the targeted hybrid-capture panel (see Methods: DNA Library Construction and Sequencing).
^{c}Median non-deduplicated sequencing depth after uCAPP-Seq (see Methods: DNA Library Construction and Sequencing).
^{d}Median deduplicated sequencing depth after uCAPP-Seq (see Methods: DNA Library Construction and Sequencing).
^{e}Median length of sonicated urine cell-free DNA fragments, as determined by uCAPP-Seq (see Methods: DNA Library Construction and Sequencing).
^{f}Percentage of deduplicated reads with duplex support as determined by uCAPP-Seq (see Methods: DNA Library Construction and Sequencing).
bp = base pairs
NGS = next-generation sequencing
uCAPP-Seq = urine Cancer Personalized Profiling by Deep Sequencing

TABLE 13
Tumor tissue and germline DNA sequencing metrics.

| | Patient | | | | | | Median |
|---|---|---|---|---|---|---|---|
| | or | | | On- | | Median | fragment |
| | Healthy | Sample | NGS | target | Median | unique | length |
| ID | Donor | source^{a} | reads^{b} | rate^{c} | depth^{d} | depth^{e} | (bp)^{f} |
| 66 | Healthy | Germline | 62696195 | 66% | 11183 | 716 | 254 |
| 67 | Healthy | Germline | 66518226 | 60% | 10881 | 781 | 242 |
| 69 | Healthy | Germline | 60441652 | 65% | 10727 | 981 | 271 |
| 70 | Healthy | Germline | 68463451 | 64% | 11973 | 972 | 282 |
| 71 | Healthy | Germline | 47987510 | 66% | 8623 | 805 | 250 |
| 72 | Healthy | Germline | 59469829 | 48% | 7732 | 825 | 266 |
| 75 | Healthy | Germline | 52901050 | 65% | 9222 | 830 | 274 |
| 89 | Healthy | Germline | 52751977 | 63% | 9005 | 680 | 241 |
| 92 | Healthy | Germline | 49134879 | 65% | 8593 | 879 | 269 |
| 94 | Healthy | Germline | 62744190 | 65% | 11067 | 1060 | 285 |
| 95 | Healthy | Germline | 61708508 | 65% | 10923 | 992 | 281 |
| 98 | Healthy | Germline | 54483951 | 64% | 9387 | 929 | 275 |
| 104 | Healthy | Germline | 59470059 | 65% | 10416 | 859 | 288 |
| 106 | Healthy | Germline | 50618155 | 64% | 8808 | 888 | 280 |
| 130 | Healthy | Germline | 50390480 | 65% | 8873 | 937 | 271 |
| BC-1045 | Patient | Germline | 80155852 | 52% | 10648 | 656 | 307 |
| BC-1050 | Patient | Germline | 47732586 | 29% | 3761 | 491 | 275 |
| BC-1058 | Patient | Germline | 88216385 | 52% | 11694 | 657 | 290 |
| BC-1062 | Patient | Germline | 63855878 | 22% | 3728 | 1028 | 238 |
| BC-1063 | Patient | Germline | 82548202 | 50% | 10587 | 621 | 258 |
| BC-1064 | Patient | Germline | 45976357 | 28% | 3498 | 394 | 246 |
| BC-1065 | Patient | Germline | 58252751 | 20% | 3213 | 880 | 256 |
| BC-1069 | Patient | Germline | 53840485 | 27% | 491 | 1064 | 247 |
| BC-1071 | Patient | Tumor | 87012882 | 63% | 14638 | 1917 | 270 |
| BC-1071 | Patient | Germline | 41983397 | 31% | 3668 | 446 | 251 |
| BC-1076 | Patient | Germline | 47472877 | 33% | 4309 | 535 | 271 |
| BC-1077 | Patient | Germline | 80035043 | 49% | 10034 | 537 | 266 |
| BC-1083 | Patient | Germline | 50816662 | 32% | 4372 | 531 | 261 |
| BC-1085 | Patient | Tumor | 62351046 | 63% | 9753 | 777 | 225 |
| BC-1085 | Patient | Germline | 53321221 | 29% | 4277 | 485 | 260 |
| BC-1088 | Patient | Tumor | 109837879 | 66% | 18822 | 2070 | 259 |
| BC-1088 | Patient | Germline | 79618718 | 53% | 11918 | 1017 | 239 |
| BC-1090 | Patient | Germline | 49898037 | 28% | 3790 | 432 | 242 |
| BC-1096 | Patient | Germline | 54165553 | 27% | 3947 | 393 | 225 |
| BC-1104 | Patient | Germline | 66777514 | 53% | 10097 | 964 | 235 |
| BC-1105 | Patient | Germline | 74350224 | 50% | 9582 | 530 | 258 |
| BC-1108 | Patient | Germline | 55329481 | 23% | 3494 | 389 | 239 |
| BC-1109 | Patient | Germline | 54869415 | 27% | 3994 | 389 | 220 |
| BC-1111 | Patient | Germline | 81563526 | 42% | 9332 | 779 | 230 |
| BC-1116 | Patient | Germline | 52360249 | 27% | 3880 | 412 | 254 |
| BC-1132 | Patient | Germline | 47569564 | 28% | 3589 | 379 | 250 |
| BC-1133 | Patient | Germline | 96716658 | 44% | 11832 | 1084 | 253 |
| BC-1135 | Patient | Germline | 113353007 | 55% | 15796 | 619 | 277 |
| BC-1140 | Patient | Germline | 82737421 | 49% | 11494 | 965 | 247 |
| BC-1147 | Patient | Germline | 63991557 | 51% | 9142 | 918 | 235 |
| BC-1171 | Patient | Germline | 88816352 | 51% | 11254 | 668 | 273 |
| BC-1174 | Patient | Germline | 83408547 | 61% | 14788 | 1293 | 233 |
| BC-1182 | Patient | Germline | 87683304 | 52% | 11625 | 580 | 237 |
| BC-1185 | Patient | Germline | 58405670 | 52% | 7588 | 307 | 222 |
| BC-1186 | Patient | Tumor | 45069412 | 75% | 660 | 75 | 258 |
| BC-1186 | Patient | Germline | 102822647 | 51% | 13135 | 746 | 257 |
| BC-1188 | Patient | Germline | 83633874 | 49% | 11425 | 970 | 252 |
| BC-1196 | Patient | Germline | 46315525 | 50% | 6423 | 824 | 300 |
| BC-1197 | Patient | Germline | 40067620 | 49% | 5295 | 614 | 253 |
| BC-1198 | Patient | Germline | 43909892 | 51% | 6140 | 789 | 281 |
| BC-1202 | Patient | Germline | 43618303 | 53% | 6307 | 793 | 262 |
| BC-1203 | Patient | Germline | 48289424 | 38% | 5052 | 657 | 242 |
| BC-1205 | Patient | Germline | 49776300 | 52% | 7073 | 991 | 260 |
| BC-1206 | Patient | Tumor | 138201355 | 75% | 19503 | 2580 | 273 |
| BC-1206 | Patient | Germline | 47031765 | 50% | 6462 | 728 | 228 |
| BC-1207 | Patient | Germline | 56854882 | 43% | 6667 | 896 | 260 |
| BC-1209 | Patient | Germline | 66631578 | 42% | 7627 | 1207 | 268 |

^{a}Germline DNA was from plasma-depleted whole blood, while tumor DNA was from tumor tissue collected prior to any systemic therapy (see Methods: Biological Specimen Collection and Processing).
^{b}Number of total sequencing reads per sample after demultiplexing (see Methods: DNA Library Construction and Sequencing).
^{c}Percentage of quality-control-passed reads overlapping with the targeted hybrid-capture panel (see Methods: DNA Library Construction and Sequencing).
^{d}Median non-deduplicated sequencing depth after CAPP-Seq (see Methods: DNA Library Construction and Sequencing).
^{e}Median deduplicated sequencing depth after CAPP-Seq (see Methods: DNA Library Construction and Sequencing).
^{f}Median length of sonicated DNA fragments, as determined by CAPP-Seq (see Methods: DNA Library Construction and Sequencing).
bp = base pairs
CAPP-Seq = Cancer Personalized Profiling by Deep Sequencing
NGS = next-generation sequencing

Urine cfDNA was sequenced from localized bladder cancer patients and healthy donors to a median deduplicated depth of 811×, while germline DNA and tumor DNA were sequenced to a median deduplicated depth of 781× and 1917×, respectively (see e.g., TABLE 11-TABLE 13). Sequences were analyzed for single-nucleotide variants (SNVs) using the uCAPP-seq bioinformatics pipeline with error suppression. Briefly, sequencing reads were demultiplexed using sample-level index barcodes, mapped to the reference genome GRCh37/hg19 (February 2009), filtered for properly paired reads, filtered for bases with Phred quality score ≥30, and deduplicated using unique molecular barcodes. The molecular barcoding strategy enabled the identification of duplex-supported reads (see e.g., TABLE 11 and TABLE 12).

Duplex-Supported Variant Calling for utDNA MRD Analysis

utDNA was assessed by deep targeted sequencing of preoperative urine-derived cfDNA. Notably, utDNA analysis from urine was performed without prior sequencing of tumor tissue (i.e., tumor-naive approach). A focused MRD gene panel encompassing 49 consensus driver genes frequently mutated in bladder cancer (see e.g., TABLE 3) was used. For SNV calling from urine, the CAPP-Seq with iDES pipeline was utilized and any mutation also detected in the matched germline DNA sample was filtered out. SNVs with population frequency >0.0001 in the Genome Aggregation Database (gnomAD) were also filtered out. The identified variants were further annotated with ANNOVAR for mutation type, associated gene name, location with respect to associated gene, and amino acid change when relevant, and only those with duplex support were considered further for the MRD analysis. These duplex-supported SNVs were then categorized as silent versus non-silent mutations. Silent mutations included the following: exonic synonymous mutations, intronic mutations (except in PLEKHS1), promoter mutations (except in TERT), 50 UTR mutations (except in TBC1D12), and 30 UTR mutations. Non-silent mutations included the following: exonic nonsynonymous mutations, splicing mutations, stop codon mutations, and the aforementioned exceptions. Overall utDNA levels were finally quantified as the highest variant allele fraction among non-silent mutations with duplex support detected by CAPP-Seq per urine sample. Duplex-supported reads were required in the MRD variant calling approach in order to significantly reduce the background error rate. The MRD gene panel, spanning 145 kb and consisting of 49 consensus driver genes, was bioinformatically tested on 2 whole exome sequencing datasets of pretreatment MIBC tumors: from The Cancer Genome Atlas (TCGA, n=409) and from both the Dana-Farber Cancer Institute and Memorial Sloan Kettering Cancer Center (DFCI/MSKCC, n=50). Concordance between urine cfDNA variants and tumor tissue variants was also assessed in 5 patients with paired urine and tumor tissue available. Combined lists of non-silent mutations with duplex support identified in urine or tumor were queried among non-reference variants present in tumor and urine, respectively (see e.g., TABLE 4).

utDNA Monitoring During Neoadjuvant Chemotherapy

Serial urine samples were obtained from 2 patients prior to starting neoadjuvant chemotherapy, while on neoadjuvant chemotherapy, and on the day of surgery. While duplex-supported reads were not required for variant monitoring, background polishing was utilized using 12 healthy donor urine samples to reduce stereotypical base substitution errors. All the mutations detected after iDES were combined and then each mutation was monitored at every time point using the CAPP-Seq bioinformatic pipeline.

Inferring TMB from utDNA Analysis

TMB was defined as the total number of non-silent mutations in the whole exome. An expanded custom hybrid-capture panel (called “TMB panel” in this study) was used to infer TMB in the cohort. The TMB panel is 387 kb in size, covers 536 genes, and spans all regions in the previously published uCAPP-Seq panel along with canonical DNA damage response (DDR) genes (see e.g., TABLE 9). To establish the relationship between the number of mutations observed in the uCAPP-Seq TMB panel to exome-wide TMB in bladder cancer, somatic variant calls were downloaded from 409 MIBC tumors that underwent whole exome sequencing by TCGA. Non-silent mutations in TCGA whole exome sequencing data were defined as the following mutation types: frameshift mutation (deletion/insertion), missense mutation, nonsense mutation, nonstop mutation, and splice mutation (region/site). In-frame mutations (deletion/insertion) were filtered out and mutations in the 30 flank, 30 UTR, 50 flank, 50 UTR, and intronic regions. TMB panel versus whole exome sequencing mutational loads were compared using Pearson correlation. This simple linear regression equation was used to infer the TMB levels from urine for all utDNA MRD-positive patients in the cohort, which was applied after accounting for potential dropout in utDNA results.

Identifying Actionable Mutations from utDNA Analysis

utDNA variants detected within the MRD gene panel after uCAPP-Seq were queried in the OncoKB database to identify patients who may harbor a clinically actionable mutation (see e.g., TABLE 6). Level 3A evidence denotes compelling clinical data supporting this mutation as a biomarker predictive of response to a drug in bladder cancer. Level 3B evidence denotes a mutation as a biomarker predictive of response to a Food and Drug Administration (FDA)-approved drug or an investigational drug in another malignancy.

Power and Statistical Analyses

Previous clinical studies in MIBC have shown that patients who do not attain a pCR experience significantly increased risk of progression and death due to residual disease compared to patients with pCR. Thus, it was hypothesized that there is a large difference in utDNA levels between patients with pCR compared to those without pCR. Based on a large effect size estimated by Cohen's f=0.5, at least 14 subjects were required in each group to detect a difference with 80% power, as determined by a 1-way ANOVA with a significance level of 0.05. Therefore, paired urine and blood samples were collected from healthy donors and bladder cancer patients with enrollment until at least 14 subjects were reached for each subgroup of the analysis.

Patient characteristics were statistically compared between groups of pCR and no pCR patients using Fisher exact test for categorical variables, Student t test for normally distributed continuous variables, and Mann-Whitney U test for non-normally distributed continuous variables. Normality was assessed using the Shapiro-Wilk test with a 0.05 significance level.

utDNA levels in each group of cohort subjects represented distributions of continuous variables and were compared without assuming normality using the Mann-Whitney U test with a 0.017 significance level, adjusted with a Bonferroni correction for comparison of 3 groups. For the analysis restricted to patients who received neoadjuvant chemotherapy, utDNA levels were compared using the Mann-Whitney U test with a 0.05 significance level. To assess utDNA as a classifier of pathologic response, receiver operating characteristic (ROC) analysis was performed and the area under the curve was measured. MRD detection was determined by identifying the utDNA level threshold on the ROC curve that classified patients with no pCR versus healthy adults with the highest Youden index. Then, this optimal threshold (utDNA level of 2.3%) was applied to establish utDNA MRD detection among patients with pCR or no pCR. Fisher exact test with a 0.017 significance level, adjusted with a Bonferroni correction for comparison of multiple proportions, was used to assess the association between utDNA MRD detection and pathologic response.

Leave-one-out cross-validation was performed in R using the caret package's LOOCV method to assess the generalizability of the utDNA MRD predictor to independent data. This was done by generating a univariate logistic regression to predict pCR based on utDNA MRD from k−1 samples and applying this model to predict the kth sample over k iterations. Accuracy of prediction was determined using the confusionMatrix function with p-value computed using the 1-sided binomial test function.

Kaplan-Meier analysis with log-rank test was performed for PFS and OS based on followup within 200 days of surgery. Multivariate logistic regression analysis was performed using the g/m function in R for predicting the absence of pCR using the following covariates: utDNA MRD detection, smoking status, neoadjuvant chemotherapy, staging at the time of diagnosis or transurethral resection of bladder tumor, age (continuous variable), sex, ethnicity, and histology. All statistical analyses were performed using R v3.6.3 through the RStudio v1.1.463 environment (RStudio, Boston, Massachusetts, USA) and Prism 8 (GraphPad Software, San Diego, California, USA).

1-82. (canceled)

83. A method of detecting urine tumor DNA (utDNA) in a subject having or suspected of having a urinary tract-associated cancer, the method comprising:
providing a biological sample comprising urine or derived from urine from the subject; and
detecting a level of utDNA in the biological sample.

84. The method of claim 83, wherein detecting a level of utDNA comprises detecting a mutation in one or more minimal residual disease (MRD)-associated genes or one or more tumor mutational burden (TMB)-associated genes.

85. The method of claim 84, wherein the mutation in the one or more MRD-associated genes or TMB-associated genes is a non-silent mutation.

86. The method of claim 83, wherein the level of utDNA is detected in the biological sample of the subject after the subject has received a neoadjuvant chemotherapy but prior to surgical intervention.

87. The method of claim 86, wherein the level of utDNA detected correlates with pathologic response to the neoadjuvant chemotherapy.

88. The method of claim 84, wherein the one or more MRD-associated genes are selected from the group consisting of TERT, TP53, and a combination thereof.

89. The method of claim 84, wherein the one or more MRD-associated genes are selected from the group consisting of
ARID1A; CDKN1A; ERCC5; KMT2C; POLE; TP53; ASXL2; CDKN2A; FANCC; KMT2D; RB1; TSC1; ATM; CREBBP; FBXW7; KRAS; RHOA; TXNIP; ATR; ELF3; FGFR3; MDM2; RHOB; ZFP36L1; BRCA1; EP300; FOXA1; NF1; RXRA; BRCA2; ERBB2; FOXQ1; NFE2L2; SPTAN1; BTG2; ERBB3; HRAS; PAIP1; STAG2; CCND1; ERBB4; KDM6A; PIK3CA; TBC1D12; CCND3; ERCC2; KLF5; PLEKHS1; TERT; and combinations thereof.

90. The method of claim 84, wherein
if one or more mutations or non-silent mutations in the one or more MRD-associated genes are detected, the subject is considered utDNA MRD-positive; or
if no mutations or non-silent mutations in the one or more MRD-associated genes are detected, the subject is considered utDNA MRD-negative.

91. The method of claim 83, further comprising calculating a threshold using utDNA values from healthy subjects or pathologic complete response (pCR) subjects; and wherein
a subject with a utDNA level above or below the threshold is designated as utDNA MRD-positive or utDNA MRD-negative, respectively.

92. The method of claim 91, wherein
the subject is designated as utDNA MRD-positive and no pCR if the utDNA level is at or above 2.3%; or
the subject is designated as utDNA MRD-negative and pCR if the utDNA level is below 2.3%.

93. The method of claim 83, wherein the biological sample comprises a urine cell-free DNA (cfDNA) sample.

94. The method of claim 83, wherein the urinary tract-associated cancer is selected from the group consisting of a cancer of the urinary tract; a bladder cancer; localized bladder cancer; metastatic bladder cancer; muscle-invasive bladder cancer (MIBC); non-muscle invasive bladder cancer (NMIBC); urothelial carcinoma; papillary urothelial carcinoma; squamous cell carcinoma; urothelial carcinoma with trophoblastic differentiation or choriocarcinomatous differentiation; a urothelial carcinoma in the urinary tract; a genitourinary cancer; kidney cancer; testicular cancer; urethral cancer; prostate cancer; penile cancer; ureteral cancer; adrenal cancer or adrenocortical carcinoma; urachal cancer; collecting duct carcinoma; renal cell carcinoma; rhabdoid tumor of the kidney; renal pelvic cancer; Wilms tumor or nephroblastoma; testicular germ cell tumor or seminoma, non-seminoma, choriocarcinoma proud or teratoma; clear cell, papillary, or chromophobe sarcomatoid renal cell carcinoma; renal medullary carcinoma; renal sarcoma; renal lymphoma; oncocytoma; angiomyolipoma; and combinations thereof.

95. The method of claim 84, wherein the one or more TMB-associated genes are selected from the group consisting of:
ABCA6; C16orf71; COLGALT2; ERBB3; HCG4P5; KCNH6; ABCC5; C1QTNF1; COPB2; ERBB4; HCN1; KCTD19; ACAA1; C3orf17; COPG1; ERCC2; HECW1; KDM3A; ACPP; C3orf20; CPD; ERCC5; HELZ; KDM6A; ACTL8; C3orf58; CPSF6; FAM71A; HERC1; KIAA0100; ADAMTS12; C3orf70; CRB1; FAM71F1; HEXIM1; KIAA0556; ADAMTS16; C9orf156; CREBBP; FANCC; HHIP; KIAA1045; ADRA1A; C9orf64; CRTC2; FANCM; HHLA2; KLF5; AHR; CACNA1A; CRYAB; FBXL7; HIST1H1B; KLF6; AKT1; CACNA2D4; CSN3; FBXO42; HIST1H1C; KMT2C; ALAS1; CACNG4; CSPG5; FBXW7; HIST1H1E; KMT2D; ALDH16A1; CADM2; CTNNA2; FCRL3; HIST1H2AG; KRAS; ALYREF; CARD11; CTNNB1; FERD3L; HIST1H2AK; KRIT1; AMACR; CARD14; CTNND2; FETUB; HIST1H2BG; LAX1; AMIGO3; CBLB; CTTN; FFAR4; HIST1H2BH; LHFPL5; ANAPC11; CCDC129; CUBN; FGFR1; HIST1H2BI; LILRA2; ANKRD28; CCDC173; CUL1; FGFR2; HIST1H2BK; LILRB4; ANO4; CCDC36; CYP7A1; FGFR3; HIST1H3B; LIPH; APOA4; CCDC96; DACH1; FLRT2; HIST1H3F; LPAR6; AQP7; CCND1; DCAF12L2; FOXA1; HIST1H3H; LRGUK; ARHGEF16; CCND3; DCAF4L2; FOXA3; HIST1H31; LRRC7; ARID1A; CCNE1; DDX42; FOXQ1; HIST1H4D; LRRK2; ARID4A; CCR5; DGCR8; FURIN; HIST1H41; LRRN1; ASH1L; CD1D; DGKG; FZD6; HIST1H4L; LRRTM1; ASIC2; CD5L; DHPS; GABRA1; HLA-A; LRTM2; ASXL2; CD86; DICER1; GABRB1; HLA-DQB2; MAG11; ATM; CDC27; DIDO1; GABRG1; HNRNPA2B1; MAGI2; ATP10A; CDH1; DLC1; GALNT8; HOXA13; MAP1LC3A; ATP8B4; CDH10; DLEC1; GALR2; HOXA7; MAPK8; ATR; CDH6; DLGAP1; GAREM; HOXB1; MAPKAPK3; ATXN2L; CDK12; DLGAP5; GATA3; HOXB3; MARCHF1; AUTS2; CDK6; DNAH10; GCNT2; HOXB4; MB21D2; BCAS3; CDKN1A; DNAH11; GEMIN2; HRAS; MCL1; BCL2L1; CDKN2A; DNAH5; GHSR; HRH1; MCM7; BIRC6; CDKN2B; DNTT; GLI3; HSPB2; MDM2; BMS1; CEP63; DOPEY2; GMPPB; HSPB2-C11orf52; MED12; BPIFB4; CEP89; DPYS; GNA13; HTR1A; MED13; BRAF; CFL1; DSC1; GNAT1; HTRA2; MET; BRCA1; CHD2; DTX1; GNGT1; ID1; METTL3; BRCA2; CLASP2; E2F3; GOLGA1; IDH1; MGAT5B; BRINP3; CLCNKB; EEF1B2; GPC5; IDH2; MIOS; BRIP1; CLIP1; EFCAB5; GPC6; IKZF2; MIR1306; BRMS1; CLVS2; EGFR; GRIK3; IRX1; MIR4728; BTG2; CNKSR3; EIF6; GRM7; ITGA3; MLIP; C10orf90; CNTN1; ELF3; GSS; ITIH3; MMP25; C11orf16; CNTN6; EMILIN2; GTPBP8; JAK2; MMP9; C11orf65; CNTNAP1; EP300; GUCY1A3; KARS; MOCS1; C12orf43; COL11A2; EPHA3; GYS1; KCNA1; MPHOSPH8; C12orf66; COL1A2; EPHB4; H1FOO; KCNB2; MROH2B; C15orf52; COL6A3; ERBB2; HARB11; KCND2; MRPS22; MS4A3; PCDHGA5; RBM47; SUMF1; WAC; MTAP; PCDHGB1; RBP4; SYNE1; WDR64; MTOR; PCDHGB2; RECQL4; SYNE2; WNT7A; MYC; PCDP1; RECQL5; SYT10; XKR7; MYCL; PCF11; RHOA; SYT15; YAE1D1; MYF5; PCK1; RHOB; T; YAP1; MYH3; PCSK6; RHOU; TADA2B; YWHAZ; MYH7; PDE1C; RIPK4; TBC1D12; ZACN; MYO3A; PDE3A; RIT2; TBC1D16; ZBTB22; NAB2; PDE4DIP; RNASE3; TBKBP1; ZBTB45; NACA2; PEG3; RNF10; TCFL5; ZBTB7B; NANOS3; PGAP3; RNF111; TENM3; ZFC3H1; NCKAP5; PHACTR1; RNF123; TERT; ZFHX4; NCOR1; PHLDA3; RNF213; TFEC; ZFP36L1; NDUFAF6; PIGS; RNF216; TFP12; ZFP36L2; NEURL4; PIK3CA; RNF220; TGIF2LX; ZFYVE1; NF1; PKHD1; ROBO2; THAP2; ZFYVE9; NFE2L2; PKHD1L1; RTN4RL2; THSD7B; ZIC1; NKIRAS2; PLAUR; RUSC1; TIE1; ZIM2; NKTR; PLEKHA7; RXRA; TMCO2; ZIM3; NKX2-2; PLEKHS1; RYR2; TMCO4; ZNF263; NLRP8; POLDIP2; S1PR1; TMEM131; ZNF276; NOM1; POLE; SAMD4B; TMEM143; ZNF277; NOS3; POLN; SATB2; TMEM200A; ZNF385D; NOTCH4; POM121L12; SCAMP3; TNXB; ZNF394; NR1D2; POT1; SCN4A; TOX4; ZNF423; NRAS; PPARG; SEMA3D; TP53; ZNF445; NRG3; PPCS; SERPINA12; TP531NP1; ZNF511; NUTM1; PPP2R1B; SF3B1; TP63; ZNF536; OTOF; PRICKLE1; SGOL1; TPRG1; ZNF608; PAIP1; PRKAA2; SHOX2; TRHR; ZNF620; PCDH10; PRMT8; SIGLEC8; TRIM42; ZNF671; PCDH17; PSD2; SLAMF1; TRIM72; ZNF703; PCDH9; PSPH; SLC22A12; TRPC4; ZNF789; PCDHA1; PTCHD2; SLC32A1; TRPS1; ZSCAN22; PCDHA10; PTEN; SLC38A2; TSC1; ZZEF1; PCDHA11; PTPN22; SLC6A15; TSHZ3; PCDHA12; PTPN9; SLC6A20; TTC18; PCDHA2; PTPRK; SLTM; TTC19; PCDHA3; PTRF; SMARCA2; TUBGCP2; PCDHA4; PVRL4; SMARCA4; TXNIP; PCDHA5; PXDN; SNX22; UBL4A; PCDHA6; PXDNL; SNX32; UPF2; PCDHA7; PYDC1; SOX4; UVSSA; PCDHA8; RAB11FIP2; SOX5; VANGL1; PCDHA9; RAD51C; SPEG; VAT1; PCDHGA1; RARG; SPSB2; VCAM1; PCDHGA2; RB1; SPTAN1; VSX1; PCDHGA3; RBM10; STAG2; VWA8; PCDHGA4; RBM26; SUCNR1; VWF; and combinations thereof.

96. The method of claim 84, wherein an inferred tumor mutational burden (iTMB) is calculated from a number of silent or non-silent mutations in the one or more TMB-associated genes, and a subject is determined to have high itMB if greater than 170 non-silent mutations in the one or more TMB-associated genes are detected per exome or a subject is determined to have low iTMB if 170 or fewer non-silent mutations in the one or more TMB-associated genes are detected per exome.

97. The method of claim 90, wherein
if the subject is considered utDNA MRD-positive, the subject is predicted to be at higher risk for no pathologic complete response (pCR); or
if the subject is considered utDNA MRD-negative, the subject is predicted to have pathologic complete response (pCR).

98. The method of claim 83, wherein the subject is receiving or has received cancer treatment and a detected reduction in MRD-associated utDNA during or after treatment is predictive of response to treatment or an increase in MRD-associated utDNA during or after treatment is predictive of development of treatment resistance.

99. The method of claim 90, wherein the subject considered utDNA MRD-negative is administered bladder-sparing treatment.

100. The method of claim 90, wherein the subject considered utDNA MRD-negative is not administered a radical cystectomy.

101. The method of claim 90, wherein if the subject is considered utDNA MRD-positive, and the one or more detected mutations comprises
a mutation in FGFR, the subject is administered erdafitinib;
a mutation in ERCC2, the subject is administered cisplatin-based chemotherapy; or
a mutation in PIK3CA and TSC1, the subject is administered PI3K inhibition therapy or mTOR inhibition therapy.

102. The method of claim 96, wherein the subject determined to have high iTMB is administered immunotherapy.
